(19) United States
(12) Patent Application Publication
(10) Pub. No.: US 2008/0193405 A1
(43) Pub. Date: Aug. 14, 2008

(54) Use of a Water-In-Water Emulsion Polymers in the Form of a Thickener for Cosmetic Preparations

(75) Inventors: Pilakesh Mukherjee, Mannheim (DE); Klemens Mathauer, Heidelberg (DE); Claudia Wood, Weinheim (DE); Matthias Laubender, Schifferstadt (DE); Ivette Garcia Castro, Ludwigshafen Gartenstadt (DE); Volker Wendel, Frankfurt (DE); Helmuth Vollmar, Ludwigshafen (DE); Audrey Renoncourt, Ludwigshafen (DE)

Correspondence Address: CONNOLLY BOVE LODGE & HUTZ LLP, 1875 EYE STREET, N.W., SUITE 1100, WASHINGTON, DC, 20036, US

(73) Assignee: BASF Aktiengesellschaft, Ludwigshafen (DE)

(21) Appl. No.: 11/814,988

(22) PCT Filed: Jan. 24, 2006
(86) PCT No.: PCT/EP2006/050420

§ 371 Date: Jul. 27, 2007

(30) Foreign Application Priority Data
Jan. 28, 2005 (DE) 102005004298.8
Dec. 23, 2005 (EP) 05112973.2

(51) Int. Cl. A61K 8/81; A61Q 5/00; A61Q 19/00

(52) U.S. Cl. 424/70.16; 514/772.4

(57) ABSTRACT

The present invention relates to the use of, if appropriate present in the form of an aqueous dispersion, polymers of ethylenically unsaturated anionic monomers for modifying the rheology of aqueous, alcoholic or aqueous/alcoholic cosmetic or dermatological compositions. The polymers are obtainable by free-radical emulsion copolymerization of water-soluble inorganic monomers in aqueous phase in the presence of, in each case, at least one stabilizing polymer of groups a) and b). The emulsion polymers are exceptionally suitable for thickening cosmetic or dermatological preparations based either on water or on alcohols.

The present invention relates to the use of, if appropriate present in the form of an aqueous dispersion, polymers of ethylenically unsaturated anionic monomers for modifying the rheology of aqueous, alcoholic or aqueous/alcoholic cosmetic or dermatological compositions.

Polymers are used widely in cosmetics. Their task in hair cosmetics consists in influencing the properties of the hair, in particular giving the hair hold, improving the combability and imparting a pleasant feel to the touch.

Thus, conditioners are used for improving the dry and wet combability, feel, shine and appearance, and to impart antistatic properties to the hair. Preference is given to using water-soluble polymers with polar, often cationic functionalities which have a greater affinity to the surface of hair, which is negative as a consequence of its structure. The structure and mode of action of various hair-treatment polymers are described in Cosmetic & Toiletries 103 (1988) 23. Standard commercial conditioner polymers are, for example, cationic hydroxyethylcellulose, cationic polymers based on N-vinylpyrrolidone, e.g. copolymers of N-vinylpyrrolidone and quaternized N-vinylimidazole, acrylamide and diallyldimethylammonium chloride or silicones.

The combination of different properties, such as, for example, strong hold, pleasant feel of the hair and simultaneous thickening effect of the polymers often presents difficulties in the hair cosmetic preparations.

This is of significance particularly in gel formulations. Moreover, customary setting polymers exhibit incompatibilities with thickener polymers, resulting in cloudiness and precipitations in the cosmetic formulations. Classic thickeners often have the disadvantage that, on account of the crosslinking, they do not form films suitable for setting hair. They ensure the consistency of the gel, but are no longer required after the gel has dried on the hair and thus potentially disrupt the application properties of the formulation (setting effect, moisture sensitivity).

Thickeners are used widely in the field of pharmacy and cosmetics for increasing the viscosity of aqueous preparations. Examples of thickeners which are used often are fatty acid polyethylene glycol monoesters, fatty acid polyethylene glycol diesters, fatty acid alkanolamides, oxyethylated fatty alcohols, ethoxylated glycerol fatty acid esters, cellulose ethers, sodium alginate, polyacrylic acids, and neutral salts.

Polymers comprising carboxyl groups are known as thickeners. These include homopolymers and copolymers of monoethylenically unsaturated carboxylic acids, such as acrylic acid, methacrylic acid, maleic acid, maleic anhydride and itaconic acid. These polymers are often crosslinked at least to a small extent. Such polymers are described, for example, in U.S. Pat. No 2,798,053, U.S. Pat. No. 3,915,921, U.S. Pat. No. 3,940,351, U.S. Pat. No. 4,062,817, U.S. Pat. No. 4,066,583, U.S. Pat. No. 4,267,103, U.S. Pat. No. 5,349,030 and U.S. Pat. No. 5,373,044.

Frequent disadvantages of these polymers when used as thickeners are their pH dependency and hydrolytic instability. In addition, large amounts of the polymers are often required in order to achieve the desired thickening effect and the stability of the preparations in the presence of electcrolytes is low.

Naturally occurring materials such as casein, alginates, methylcellulose, hydroxyethylcellulose, hydroxypropylcellulose and carbomethoxycellulose are also used as thickeners. These have, inter alia, the disadvantage of sensitivity toward microbiological factors and consequently require the addition of biocids.

DE-A 103 38 828 discloses aqueous dispersions of water-soluble, anionic polymers which are obtainable by free-radical polymerization of ethylenically unsaturated anionic monomers in aqueous medium in the presence of at least one stabilizer, where the stabilizer used is at least one water-soluble polymer from the groups
(a) graft polymers of vinyl acetate and/or vinyl propionate on polyethylene glycols, polyethylene glycols terminally capped at one or both ends with alkyl, carboxyl or amino groups, copolymers of alkylpolyalkylene glycol acrylates or alkylpolyalkylene glycol methacrylates and acrylic acid and/or methacrylic acid, polyalkylene glycols with molar masses M_{N} of from 1000 to 100 000, polyalkylene glycols terminally capped at one or both ends with alkyl, carboxyl or amino groups having molar masses M_{N} of from 1000 to 100 000
and
(b) hydrolyzed copolymers of vinyl alkyl ethers and maleic anhydride in the form of the free carboxyl groups and in the form of the salts neutralized at least partially with alkali metal hydroxides or ammonium bases and/or a water-soluble starch from the group of cationically modified potato starch, anionically modified potato starch, degraded potato starch and maltodextrin.

The aqueous dispersions are used as thickeners for aqueous systems such as paper coating compositions, pigment printing pastes, cosmetic formulations and leather treatment compositions. The use of these polymers in cosmetic gel preparations, cosmetic cleansing compositions, such as, for example, shampoos, or skin cosmetic preparations is not described.

The unpublished application DE-A 10 2004 038 983.7 likewise discloses aqueous dispersions of water-soluble and/or water-swellable anionic polymers which are obtainable by free-radical polymerization of ethylenically unsaturated, anionic monomers in aqueous medium in the presence of at least one stabilizer, where the polymerization is carried out in the presence of at least one water-soluble polymer of groups
(a) graft polymers of vinyl acetate and/or vinyl propionate on (i) polyethylene glycols or (ii) polyethylene glycols or polypropylene glycols terminally capped at one or both ends with alkyl, carboxyl or amino groups, polyalkylene glycols, polyalkylene glycols terminally capped at one or both ends with alkyl, carboxyl or amino groups, and
and
(b) water-soluble copolymers of
(b1) nonionic monoethylenically unsaturated monomeres,
(b2) cationic monoethylenically unsaturated monomers and, if appropriate,
(b3) anionic monoethylenically unsaturated monomers, where the fraction of copolymerized cationic monomers is greater than that of the anionic monomers,
   as stabilizer. The use of these polymers in cosmetic gel preparations, cosmetic cleansing compositions such as, for example, shampoos, or skin cosmetic preparations is not described.

It was an object of the present invention to find rheology-modifying, in particular thickening, polymers which are highly suitable for cosmetic applications and have good application properties particularly in the field of skin and hair cosmetics. These include, besides the good thickening effect for a low material feed over a broad pH range, also clarity in the case of gel applications, (co)emulsifying and stabilizing effect for water-insoluble and/or difficult-to-stabilize components, such as silicones and enzymes, hydrolysis- and/or oxidation-sensitive substances, compatibility with cosmetically customary polymers, such as, for example, cationic polymers, good incorporability into cosmetic preparations, compatibility with high surfactant contents, for example in cosmetic cleansing compositions such as shampoos.

For gels in particular, the highest possible transparency (clarity) of the preparations is desired.

Cosmetic preparations are generally aqueous, alcoholic or mixed aqueous-alcoholic in nature. It is therefore very particularly desired to provide thickeners which allow the rheology of preparations based either on alcohol or on water to be adjusted over a wide pH range.

The object is achieved through the use of polymers, if appropriate present in the form of an aqueous dispersion, of ethylenically unsaturated anionic monomers obtainable by free-radical polymerization of the monomers in aqueous medium, where the polymerization is carried out in the presence of, in each case, at least one polymer chosen from group a) and at least one polymer chosen from group b), where group a) consists of

a1) graft polymers of vinyl acetate and/or vinyl propionate on (i) polyethylene glycols or (ii) polyethylene glycols or polypropylene glycols terminally capped at one or both ends with alkyl, carboxyl or amino groups,

a2) polyalkylene glycols,

a3) polyalkylene glycols terminally capped at one or both ends with alkyl, carboxyl or amino groups,

a4) copolymers of alkyl polyalkylene glycol (meth)acrylates and (meth)acrylic acid, and where group b) consists of

b1) at least partially hydrolyzed copolymers of vinyl alkyl ethers and maleic anhydride, which may be present at least partially in salt form,

b2) water-soluble starch chosen from the group consisting of cationically modified starch, anionically modified starch, degraded starch and maltodextrin,

b3) anionic copolymers chosen from the group of
homopolymers and copolymers of anionic monomers,
copolymers of anionic and cationic and, if appropriate, neutral monomers, where the fraction of copolymerized anionic monomers is greater than that of cationic monomers, and
copolymers of at least one anionic monomer and at least one monomer from the group of esters of anionic monomers with monohydric alcohols, styrene, N-vinylpyrrolidone, N-vinylcaprolactam, N-vinylimidazole, N-vinylformamide, acrylamide, methacrylamide, vinyl acetate and vinyl propionate,

b4) cationic copolymers of nonionic monoethylenically unsaturated monomers and cationic monoethylenically unsaturated monomers and, if appropriate, anionic monoethylenically unsaturated monomers, where in every case the number of cationic groups is greater than the number of anionic groups,

for modifying the rheology of aqueous, alcoholic or aqueous/alcoholic cosmetic or dermatological compositions.

The W/W emulsion polymers of ethylenically unsaturated anionic monomers, if appropriate present in the form of an aqueous dispersion, suitable for the use according to the invention are sometimes referred to below as “emulsion polymers according to the invention”, or “W/W polymers” or “W/W emulsion polymers”. The polymers of groups a) and b) are also referred to below as “stabilizers”.

It is preferred that the W/W emulsion polymers and the polymers a) and polymers b), also referred to as stabilizers, are water-soluble. Water-soluble polymers are understood here as meaning polymers which dissolve at 20° C. and at atmospheric pressure in an amount of at least 1 g in 1 liter of completely demineralized water to give a clear solution.

Suitable ethylenically unsaturated, anionic monomers are, for example, monoethylenically unsaturated C₃- to C₅-carboxylic acids, such as acrylic acid, methacrylic acid, ethacrylic acid, crotonic acid, maleic acid, fumaric acid, vinylsulfonic acid, styrenesulfonic acid, acrylamidomethylpropanesulfonic acid, vinylphosphonic acid, itaconic acid and/or the alkali metal or ammonium salts of these acids. Anionic monomers preferably used include acrylic acid, methacrylic acid, maleic acid and acrylamido-2-methylpropanesulfonic acid. Particular preference is given to aqueous dispersions of polymers based on acrylic acid. The anionic monomers can be polymerized either on their own to give homopolymers or else in a mixture with one another to give copolymers. Examples thereof are the homopolymers of acrylic acid or copolymers of acrylic acid with methacrylic acid and/or maleic acid.

The polymerization of the anionic monomers can, however, also be carried out in the presence of other ethylenically unsaturated monomers. These monomers may be nonionic or else carry a cationic charge. Examples of such comonomers are acrylamide, methacrylamide, (meth)acrylic esters of monohydric C₁-C₂₂-alcohols, C₃-C₂₂-alkyl vinyl ethers, C₆-C₁₆-olefins, polyisobutene derivatives, vinyl acetate, vinyl propionate, dialkylaminoethyl (meth)acrylates, dialkylaminopropyl (meth)acrylates, diallyldimethylammonium chloride, N-vinylformamide, vinylimidazole and quaternized vinylimidazole and partially or completely neutralized or quaternized dialkylamininoalkyl(meth)acrylamides.

Through the copolymerization with hydrophobic monomers such as (meth)acrylic esters of monohydric alcohols having 4 to 22 carbon atoms, C₃-C₂₂-alkyl vinyl ethers, C₆-C₁₆-olefins or polyisobutene derivatives, it is possible to increase the tolerance to salts (salt stability) of the cosmetic or dermatological preparations thickened with the W/W emulsion polymers.

Basic monomers, such as dialkylaminoalkyl (meth)acrylates, e.g. dimethylaminoethyl acrylate or dimethylaminoethyl methacrylate, can be used in the polymerization either in the form of the free bases or else in partially or completely neutralized form or in a form quaternized, for example, with C₁- to C₁₈-alkyl halides. The comonomers are used in the preparation of the anionic polymers, for example, in amounts such that the resulting polymers are water-soluble and have an anionic charge. Based on the monomers used overall in the polymerization, the amount of nonionic and/or cationic comonomers is, for example, 0 to 99% by weight, preferably 5 to 75% by weight.

Preferred copolymers are, for example, copolymers of 25 to 90% by weight of acrylic acid and 75 to 10% by weight of acrylamide. Particular preference is given to homopolymers of acrylic acid which are obtainable by free-radical polymerization of acrylic acid in the absence of other monomers, and copolymers of acrylic acid and/or methacrylic acid which can be prepared by copolymerization of acrylic acid and/or methacrylic acid in the presence of pentaerythritol triallyl ether, N,N′-divinylethyleneurea, methylenebisacrylamide, esters of dihydric alcohols having 2 to 8 carbon atoms and C₃- to C₅-carboxylic acids, ethoxylated trimethylolpropane triacrylate, ethoxylated trimethylolpropane trimethylacrylate, pentaerythritol triacrylate, pentaerythritol tetraacrylate, triallylmethylammonium chloride, allyl ethers of sugars comprising at least two allyl groups, vinyl ethers having at least two vinyl groups, or triallylamine, and mixtures of these compounds.

The polymerization can thus be carried out in the presence of at least one crosslinker. This then gives W/W polymers with a higher molar mass than in the case of polymerization of the anionic monomers in the absence of a crosslinker. Moreover, the incorporation of a crosslinker into the polymers leads to reduced solubility of the polymers in water. Depending on the amount of copolymerized crosslinker, the polymers become water-insoluble, but are swellable in water. Between complete solubility of the polymers in water and swelling of the polymers in water there are fluid transitions. On account of their swelling capacity in water, crosslinked polymers have a high water absorption capacity.

Crosslinkers which can be used are all compounds which have at least two ethylenically unsaturated double bonds in the molecule. Such compounds are used, for example, in the preparation of crosslinked polyacrylic acids as superabsorbent polymers, cf. EP-A 858 478, page 4, line 30 to page 5, line 43. Examples of crosslinkers are triallylamine, pentaerythritol triallyl ether, methylenebisacrylamide, N,N′-divinylethyleneurea, allyl ethers comprising at least two allyl groups or vinyl ethers, having at least two vinyl groups, of polyhydric alcohols such as, for example, sorbitol, 1,2-ethanediol, 1,4-butanediol, trimethylolpropane, glycerol, diethylene glycol and of sugars such as sucrose, glucose, mannose, dihydric alcohols having 2 to 4 carbon atoms and completely esterified with acrylic acid or methacrylic acid, such as ethylene glycol dimethacrylate, ethylene glycol diacrylate, butanediol dimethacrylate, butanediol diacrylate, diacrylates or dimethacrylates of polyethylene glycols with molecular weights of from 300 to 600, ethoxylated trimethylolpropane triacrylates or ethoxylated tri methylolpropane trimethacrylates, 2,2-bis(hydroxymethyl)butanol trimethacrylate, pentaerythritol triacrylate, pentaerythritol tetraacrylate and triallylmethylammonium chloride. If, in the preparation of the anionic dispersions, crosslinkers are used, then the amounts of crosslinker used in each case are, for example, 0.0005 to 5.0% by weight, preferably 0.001 to 1.0% by weight, based on the monomers used overall in the polymerization. Preferably used crosslinkers are pentaerythritol triallyl ether, N,N′-divinylethyleneurea, allyl ethers, comprising at least two allyl groups, of sugars such as sucrose, glucose or mannose and triallylamine and/or ethoxylated trimethylolpropane triacrylate, and mixtures of these compounds.

The polymerization can additionally be carried out in the presence of at least one chain-transfer agent. This then gives polymers which have a lower molar mass than polymers prepared without chain-transfer agents. Examples of chain-transfer agents are compounds which comprise sulfur in bonded form, such as dodecyl mercaptan, thiodiglycol, ethylthioethanol, di-n-butyl sulfide, di-n-octyl sulfide, diphenyl sulfide, diisopropyl disulfide, 2-mercaptoethanol, 1,3-mercaptopropanol, 3-mercaptopropane-1,2-diol, 1,4-mercaptobutanol, thioglycolic acid, 3-mercaptopropionic acid, mercaptosuccinic acid, thioacetic acid and thiourea, aldehydes, organic acids, such as formic acid, sodium formate or ammonium formate, alcohols, such as, in particular, isopropanol, and phosphorus compounds, e.g. sodium hypophosphite. In the polymerization it is possible to use a single chain transfer agent or two or more chain transfer agents. If they are used in the polymerization, they are used, for example, in an amount of from 0.01 to 5.0% by weight, preferably 0.2 to 1% by weight, based on the total monomers. The chain-transfer agents are preferably used together with at least one crosslinker in the polymerization. By varying the amount and the ratio of chain-transfer agent and crosslinker, it is possible to control the rheology of the resulting polymers. During the polymerization, chain-transfer agents and/or crosslinkers can be initially introduced, for example in the aqueous polymerization medium, or be metered into the polymerization mixture together with or separately from the monomers according to the progress of the polymerization.

In the polymerization, use is usually made of initiators which form free radicals under the reaction conditions. Suitable polymerization initiators are, for example, peroxides, hydroperoxides, hydrogen peroxide, sodium persulfate or potassium persulfate, redox catalysts and azo compounds, such as 2,2-azobis(N,N-dimethyleneisobutyramidine)dihydrochloride, 2,2-azobis(4-methoxy-2,4-dimethylvaleronitrile), 2,2-azobis(2,4-dimethylvaleronitrile) and 2,2-azobis(2-amidinopropane)dihydrochloride). The initiators are used in the amounts customary in the polymerization. Preference is given to using azo initiators as polymerization initiators. However, the polymerization can also be initiated using high-energy rays such as electron rays or by irradiation with UV light.

The aqueous dispersions of the preferably water-soluble anionic W/W emulsion polymers have a polymer concentration of anionic polymers of, for example, 1 to 70% by weight, in most cases 5 to 50% by weight, preferably 10 to 25% by weight and particularly preferably 15 to 20% by weight.

According to the invention, they comprise at least two different groups of the abovementioned polymers (a) and (b) for stabilizing the anionic polymers which form during the polymerization. The amount of stabilizers (a) and (b) in the aqueous dispersion is, for example, 1 to 40% by weight, in most cases 5 to 30% by weight and preferably 10 to 25% by weight. The aqueous dispersions have, for example at a pH of 2.5, viscosities in the range from 200 to 100 000 mPas, preferably 200 to 20 000 mPas, preferably 200 to 10 000 mPas (measured in a Brookfield viscosimeter at 20° C., spindle 6, 100 rpm).

Uncrosslinked anionic polymers which are suitable for the use according to the invention regularly have molecular weights M_{w} in the range from 10 000 to 15 million, preferably from 50 000 to 10 million g/mol. The molecular weights are determined, for example, by customary methods known to the person skilled in the art, such as SEC (size exclusion chromatography) against a polyacrylic acid standard or using FFF (field-flow fractionation).

The molecular weight of crosslinked polymers cannot be determined in this way. Their molecular weight depends on the amount of crosslinker used and the degree of branching of the polymers and can lie outside of the range given for the uncrosslinked polymers.

Polymers of Group a)

Stabilizers of group (a) include a1) graft polymers of vinyl acetate and/or vinyl propionate on (i) polyethylene glycols or (ii) polyethylene glycols or polypropylene glycols terminally capped at one or both ends with alkyl, carboxyl or amino groups, copolymers of alkylpolyalkylene glycol (meth)acrylates and (meth)acrylic acid, and also polyalkylene glycols and polyalkylene glycols terminally capped at one or both ends with alkyl, carboxyl or amino groups.

Polyalkylene glycols are described, for example, in WO 03/046024, page 4, line 37 to page 8, line 9. The polyalkylene glycols described therein can either be used directly as stabilizer of group (a), or be modified by grafting, for example, 10 to 1000, preferably 30 to 300, parts by weight of vinyl acetate and/or vinyl propionate onto 100 parts by weight of the polyalkylene glycols. Preference is given to using polyethylene glycol with a molecular weight M_{N} of from 1000 to 100 000 as graft base, and grafting vinyl acetate onto it.

Suitable stabilizers of group (a) are also a2) copolymers of alkylpolyalkylene glycol acrylates or alkylpolyalkylene glycol methacrylates and acrylic acid and/or methacrylic acid. They are prepared by firstly esterifying addition products of ethylene oxide and/or propylene oxide onto, for example, C₁- to C₁₈-alcohols with acrylic acid and/or methacrylic acid, and then copolymerizing these esters with acrylic acid and/or methacrylic acid. The copolymers usually used comprise, for example, 5 to 60% by weight, preferably 10 to 35% by weight, of copolymerized units of alkylpolyalkylene glycol (meth)acrylates and 95 to 40% by weight, preferably 90 to 65% by weight, of copolymerized units of (meth)acrylic acid. They mostly have molar masses M_{W} of from 2000 to 50 000, preferably 5000 to 20 000. These copolymers can be used in the form of the free acid groups or else in completely or partially neutralized form for the preparation of the dispersions. The carboxyl groups of the copolymers are preferably neutralized with sodium hydroxide or ammonia.

Further suitable stabilizers (a) are the polyalkylene glycols a3) already mentioned above, and the polyalkylene glycols a4) terminally capped at one or both ends with alkyl, carboxyl or amino groups. The polymers specified above have, for example, molar masses Mₙ of from 100 to 100 000, preferably from 300 to 80 000, particularly preferably from 600 to 50 000 and in particular from 1000 to 50 000.

Advantageously, the polymers of group (a) used are polyalkylene glycols with molar masses Mₙ of from 100 to 100 000, polyalkylene glycols terminally capped at one or both ends with alkyl, carboxyl or amino groups and having molar masses Mₙ of from 100 to 100 000.

Such polymers are described, for example, in the above cited WO 03/046024, page 4, line 37 to page 8, line 9. Preferred polyalkylene glycols are, for example, polyethylene glycol, polypropylene glycol and block copolymers of ethylene oxide and propylene oxide. The block copolymers can comprise copolymerized ethylene oxide and propylene oxide in any amounts and in any order. The OH end groups of the polyalkylene glycols can, if appropriate, be terminally capped at one or both ends, with alkyl, carboxyl or amino groups, in which case a methyl group is preferably suitable as end group.

Particularly preferably used stabilizers of group (a) are copolymers of ethylene oxide and propylene oxide. Preference is especially given to block copolymers of ethylene oxide and propylene oxide with a molar mass Mₙ of from 500 to 20 000 g/mol and a content of ethylene oxide units of from 10 to 80 mol %.

Particularly preferably used stabilizers of group (a) are block copolymers of the general formula (EO)ₓ(PO)_{y}(EO)_{z}. The OH end groups of these polyalkylene glycols can, if appropriate, be terminally capped at one or both ends with alkyl, carboxyl or amino groups, where a methyl group is preferably suitable as end group. The molar mass of preferred polyalkylene glycols is in the range from 300 to 20 000, preferably from 900 to 9000 g/mol, with a fraction of ethylene oxide in the range from 10 to 90% by weight. Such polyalkylene glycols are commercially available, for example, as Pluronic® grades.

The Pluronic® PE grades are low-foam, nonionic surfactants which are prepared by copolymerization of propylene oxide and ethylene oxide. As the following general formula (I) shows, the Pluronic® PE grades are block polymers in which polypropylene glycol forms the central molecular moiety:

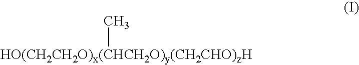

Particular preference is given to the Pluronic PE grades such as, for example, Pluronic® PE 3100, Pluronic® PE 4300, Pluronic® PE 6100, Pluronic® PE 6120, Pluronic® PE 6200, Pluronic® PE 6400, Pluronic® PE 7400, Pluronic® PE 8100, Pluronic® PE 9200, Pluronic® PE 9400, Pluronic® PE 10100, Pluronic® PE 10300, Pluronic® PE 10400, Pluronic® PE 10500, Pluronic® PE 10500 solution, Pluronic® PE 3500.

The table below gives an overview of the Pluronic® grades suitable as a).

| | | | | Fraction of |
|---|---|---|---|---|
| | | Molecular mass | | polyethylene |
| | | of the polypropylene | | glycol in the |
| Pluronic ® | Number | glycol block (g/mol) | Number | molecule (%) |
| PE 3100 | 3 | 850 | 1 | 10 |
| PE 3500 | 3 | 850 | 5 | 30 |
| PE 4300 | 4 | 1100 | 3 | 30 |
| PE 6100 | 6 | 1750 | 1 | 10 |
| PE 6120 | 6 | 1750 | 12 | 12 |
| PE 6200 | 6 | 1750 | 2 | 20 |
| PE 6400 | 6 | 1750 | 4 | 40 |
| PE 6800 | 6 | 1750 | 8 | 80 |
| PE 7400 | 7 | 2100 | 4 | 40 |
| PE 8100 | 8 | 2300 | 1 | 10 |
| PE 9200 | 9 | 2750 | 2 | 20 |
| PE 9400 | 9 | 2750 | 4 | 40 |
| PE 10100 | 10 | 3250 | 1 | 10 |
| PE 10300 | 10 | 3250 | 3 | 30 |
| PE 10400 | 10 | 3250 | 4 | 40 |
| PE 10500 | 10 | 3250 | 5 | 50 |

In a preferred embodiment of the invention, mixtures of the abovementioned polyalkylene glycols are used as polymers a). Preferred mixtures are, for example, mixtures of different Pluronic grades, where the mixing weight ratio is in the range from 5:1 to 1:5, preferably in the range from 2:1 to 1:2 and in particular in the range from 1.3:1 to 1:1.3 liegt. Of particularly good suitability for preparing the W/W emulsion polymers for the use according to the invention are mixtures which comprise Pluronic®PE 4300 and Pluronic®PE 6200, or consist thereof.

The polymers of group (a) are used in the preparation of the dispersions, for example, in amounts of from 1 to 39.5% by weight, preferably 5 to 30% by weight and particularly preferably 10 to 25% by weight, based on the total dispersion.

Polymers of Group (b)

Suitable Polymers of Group b) are Chosen From

b1) at least partially hydrolyzed copolymers of vinyl alkyl ethers and maleic anhydride, which may be present at least partially in salt form,

b2) water-soluble starch from the group of cationically modified starch, anionically modified starch, degraded starch and maltodextrin,

b3) anionic copolymers chosen from the group consisting of
homopolymers and copolymers of anionic monomers,
copolymers of anionic and cationic and, if appropriate, neutral monomers, where the fraction of copolymerized anionic monomers is greater than that of cationic monomers, and
copolymers of at least one anionic monomer and at least one monomer from the group of esters of anionic monomers with monohydric alcohols, styrene, N-vinylpyrrolidone, N-vinylcaprolactam, N-vinylimidazole, N-vinylformamide, acrylamide, methacrylamide, vinyl acetate and vinyl propionate,

b4) cationic copolymers of nonionic monoethylenically unsaturated monomers and cationic monoethylenically unsaturated monomers and, if appropriate, anionic monoethylenically unsaturated monomers, where in every case the number of cationic groups is greater than the number of anionic groups.

Suitable Polymers of Group (b) are:

b1) at least partially hydrolyzed copolymers of vinyl alkyl ethers and maleic anhydride, which may be present at least partially in the form of the alkali metal or ammonium salts. The alkyl group of the vinyl alkyl ethers preferably has 1 to 4 carbon atoms. The copolymers are obtainable by copolymerization of the vinyl alkyl ethers with maleic anhydride and subsequently partial or complete hydrolysis of the anhydride groups to carboxyl groups and, if appropriate, partial or complete neutralization of the carboxyl groups with formation of the salts. Particularly preferred polymers of group (b) are at least partially or completely hydrolyzed copolymers of vinyl methyl ether and maleic anhydride, which are present at least partially in the form of their alkali metal or ammonium salts.

b2) starches from the group of cationically modified starch, anionically modified starch, degraded starch and maltodextrin. Starches can be obtained from beans, peas, barley, oats, millet, such as, for example, wax millet, potatoes, corn, such as, for example, amylo corn or wax corn, manioc, rice, such as, for example, wax rice, rye or wheat. Preferred starches are water-soluble starches, in particular water-soluble potato starches. Examples of cationically modified potato starches are the commercial products Amylofax® 15 and Perlbond®970. A suitable anionically modified potato starch is Perfectamyl® A 4692. The modification here consists essentially in a carboxylation of potato starch. C*Pur® 1906 is an example of an enzymatically degraded potato starch and Maltodextrin C 01915 for a hydrolytically degraded potato starch. Of the specified starches, preference is given to using maltodextrin.

b3) Anionic copolymers chosen from the group of

b3-1) homopolymers and copolymers comprising or consisting of anionic monomers,

b3-2) copolymers of anionic and cationic and, if appropriate, neutral monomers, where the fraction of the copolymerized anionic monomers is greater than that of the cationic monomers and

b3-3) copolymers of at least one anionic monomer and at least one monomer from the group of esters of anionic monomers with monohydric alcohols, styrene, N-vinylpyrrolidone, N-vinylcaprolactam, N-vinylimidazole, N-vinylformamide, acrylamide, methacrylamide, vinyl acetate and vinyl propionate.

Polymers of group (b3-1) used are, for example, at least one homopolymer of an ethylenically unsaturated C₃- to C₅-carboxylic acid, vinylsulfonic acid, styrenesulfonic acid, acrylamidomethylpropanesulfonic acid, vinylphosphonic acid, their salts neutralized partially or completely with alkali metal and/or ammonium bases and/or at least one copolymer of these monomers. Examples of ethylenically unsaturated carboxylic acids which are used for the preparation of the aqueous dispersions have already been specified. These anionic monomers can accordingly likewise be used for the preparation of the polymers (b) of the stabilizer mixtures. Preferably, acrylic acid, methacrylic acid, acrylamidomethylpropanesulfonic acid and/or mixtures in any ratios are suitable here.

Of particular suitability are copolymers of methacrylic acid and acrylamidomethylpropanesulfonic acid, where, in a preferred embodiment, the molar ratio of the monomers used for preparing the copolymers, methacrylic acid to acrylamidomethylpropanesulfonic acid, is in the range from 9:1 to 1:9, preferably from 9:1 to 6:4.

Further suitable polymers of group (b3-2) of the stabilizer mixture are copolymers of
(i) at least one ethylenically unsaturated C₃- to C₅-carboxylic acid, vinylsulfonic acid, styrenesulfonic acid, acrylamidomethylpropanesulfonic acid, vinylphosphonic acid and/or alkali metal and/or ammonium salts thereof,
(ii) at least one cationic monomer from the group of partially or completely neutralized dialkylaminoalkyl (meth)acrylates, partially or completely quaternized dialkylaminoalkyl (meth)acrylates, dialkylaminoalkyl(meth)acrylamides in quaternized or neutralized form, dialkyldiallylammonium halides and quaternized n-vinylimidazole and, if appropriate,
(iii) at least one neutral monomer,
   where the fraction of the copolymerized anionic monomers is greater than that of the cationic monomers.

Examples of anionic monomers (i) have already been mentioned above.

Suitable cationic monomers (ii) are, for example, dialkylaminoalkyl (meth)acrylates, such as dimethylaminoethyl acrylate, dimethylaminoethyl methacrylate, diethylaminoethyl acrylate, diethylaminoethyl methacrylate, dimethylaminopropyl acrylate, dimethylaminopropyl methacrylate, diethylaminopropyl acrylate and diethylaminopropyl methacrylate, dialkyldiallylammoniun halides, such as dimethyldiallylammonium chloride and diethyldiallylammonium chloride, N-vinylimidazole, quaternized N-vinylimidazole and dialkylaminoalkylacrylamides, such as dimethylaminoethylacrylamide or dimethylaminoethylmethacrylamide.

Basic monomers such as dimethylaminoethyl acrylate or dimethylaminoethyl methacrylate can be used either in the form of the free bases or in a form partially or completely neutralized with acids such as hydrochloric acid, sulfuric acid, formic acid and p-toluenesulfonic acid. Furthermore, the basic monomers can be partially or completely quaternized by reaction with C₁- to C₁₈-alkyl halides and/or C₁- to C₁₈-alkyl C₁- to C₁₈-alkylaryl halides and be used in this form in the polymerization. Examples thereof are the dimethylaminoethyl (meth)acrylates completely quaternized with methyl chloride, such as dimethylaminoethyl acrylate methochloride or dimethylaminoethyl methacrylate methochloride. The polymers of group (b) can also comprise vinylamine units as cationogenic groups. Such polymers are obtainable, for example, by polymerizing N-vinylformamide, if appropriate together with at least one anionic water-soluble monomer, and then hydrolyzing the polymers with partial elimination of formyl groups to give polymers comprising vinylamine units.

Neutral monomers (iii) which can be used are, for example, the esters of anionic monomers, in particular of C₃- to C₅-carboxylic acids, and monohydric alcohols having 1 to 20 carbon atoms, such as, for example, methyl acrylate, methyl methacrylate, ethyl acrylate, n-, sec- and tert-butyl acrylate, 2-ethylhexyl acrylate, ethyl methacrylate, isopropyl acrylate, isopropyl methacrylate and n-, sec- and tert-butyl methacrylate, and acrylamide, methacrylamide, acrylonitrile, methacrylonitrile, N-vinylpyrrolidone, N-vinylimidazole, N-vinylformamide, vinyl acetate, vinyl propionate and styrene.

In the amphoteric compolymers suitable as component (b3-2), the fraction of copolymerized anionic monomers is always greater than that of the cationic monomers. These copolymers thus always carry an anionic charge.

Suitable copolymers of group (b3-3) are, furthermore, copolymers of
(i) at least one anionic monomer and
(ii) at least one monomer from the group of esters of ethylenically unsaturated acids with monohydric alcohols, styrene, N-vinylpryrrolidone, N-vinylcaprolactam, N-vinylimidazole, N-vinylformamide, acrylamide, methacrylamide, vinyl acetate and vinyl propionate
   e.g. copolymers of acrylic acid, methyl acrylate and N-vinylpyrrolidone or copolymers of methacrylic acid, acrylamidomethylpropanesulfonic acid, methyl acrylate and vinylimidazole.

The polymers (b3) can comprise, in copolymerized form, the suitable monomers in any ratios, the amphoteric copolymers merely being constructed so that they always carry an anionic charge. The average molar mass M_{w} of the polymers of group (b) of the stabilizer mixture is, for example, up to 1.5 million, in most cases up to 1.2 million and is preferably in the range from 1000 to 1 million, in most cases 1500 to 100 000 and in particular 2000 to 70 000 (determined by the light-scattering method).

b4) Cationic polymers of nonionic monoethylenically unsaturated monomers and cationic monoethylenically unsaturated monomers and, if appropriate, anionic monoethylenically unsaturated monomers, where the number of cationic groups is greater than the number of anionic groups.

As polymers of group (b4), use is made of copolymers of
(b4-1) water-soluble, nonionic, monoethylenically unsaturated monomers,
(b4-2) water-soluble, cationic, monoethylenically unsaturated monomers and, if appropriate,
(b4-3) water-soluble, anionic, monoethylenically unsaturated monomers,
   where the fraction of the copolymerized cationic monomers is greater than that of the anionic monomers.

Examples of water-soluble, nonionic monomers (b1) are acrylamide, methacrylamide, N-vinylformamide, N-vinylpyrrolidone and N-vinylcaprolactam. Of suitability in principle as monomers of group (b1) are all nonionic, monoethylenically unsaturated monomers which have a solubility in water of at least 100 g/l at a temperature of 20° C. Particular preference is given to monomers (b1) which are miscible with water in any ratio and form clear aqueous solutions, such as acrylamide or N-vinylformamide.

Water-soluble, cationic, monoethylenically unsaturated monomers (b4-2) are, for example, dialkylaminoalkyl (meth)acrylates, such as dimethylaminoethyl acrylate, dimethylaminoethyl methacrylate, diethylaminoethyl acrylate, diethylaminoethyl methacrylate, dimethylaminopropyl acrylate, dimethylaminopropyl methacrylate, diethylaminopropyl acrylate and diethylaminopropyl methacrylate, dialkyldiallylammonium halides, such as dimethyldiallylammonium chloride and diethyldiallylammonium chloride, N-vinylimidazole and quaternized N-vinylimidazole. Basic monomers such as dimethylaminoethyl acrylate or dimethylaminoethyl methacrylate can be used either in the form of the free bases or in a form neutralized partially or completely with acids, such as hydrochloric acid, sulfuric acid, formic acid and p-toluenesulfonic acid. The basic monomers can, moreover, be partially or completely quaternized by reaction with C₁- to C₁₈-alkyl halides and/or C₁- to C₁₈-alkyl C₁- to C₁₈-alkylaryl halides and be used in this form in the polymerization. Examples thereof are the dimethylaminoethyl (meth)acrylates completely quaternized with methyl chloride, such as dimethylaminoethyl acrylate methochloride or dimethylaminoethyl methacrylate methochloride. The polymers of group (b4) can also comprise vinylamine units as cationic group. Such polymers are obtainable, for example, by polymerizing N-vinylformamide, if appropriate together with at least one anionic water-soluble monomer, and then hydrolyzing the polymers with partial elimination of formyl groups to give polymers comprising vinylamine units.

The polymers of group (b4) can, if appropriate, also comprise, in copolymerized form, at least one anionic monoethylenically unsaturated monomer (b4-3). Examples of such monomers are the anionic monomers already mentioned above which form water-soluble polymers such as, for example, acrylic acid, methacrylic acid, vinylsulfonic acid, vinylphosphonic acid, maleic acid, fumaric acid, crotonic acid, itaconic acid, and the alkali metal and ammonium salts of these acids.

Examples of copolymers of group (b4) are copolymers of
(b4-1) acrylamide, methacrylamide, N-vinylformamide, N-vinylpyrrolidone and/or N-vinylcaprolactam,
(b4-2) dialkylaminoalkyl acrylate, dialkylaminoalkyl methacrylate, partially or completely neutralized dialkylaminoalkyl (meth)acrylate, quaternized dialkylaminoalkyl (meth)acrylates, dialkyldiallylammonium halides, N-vinylimidazole and quaternized N-vinylimidazole and, if appropriate,
(b4-3) acrylic acid, methacrylic acid, vinylsulfonic acid, vinylphosphonic acid, maleic acid, fumaric acid, crotonic acid, itaconic acid, and the alkali metal and ammonium salts of these acids.

The polymers (b4) comprise, for example,
(b4-1) 2 to 90 mol %, preferably 20 to 80 mol % and particularly preferably 35 to 70 mol %, of at least one nonionic monomer
(b4-2) 2 to 90 mol %, preferably 20 to 80 mol % and particularly preferably 35 to 70 mol %, of at least one cationic monomer
and
(b4-3) 0 to 48.9 mol %, preferably 0 to 30 mol % and particularly preferably 0 to 10 mol %, of at least one anionic monomer in copolymerized form, where the fraction of cationic monomer units is greater than that of the anionic monomer units.

Individual examples of polymers (b4) are copolymers of acrylamide and dimethylaminoethyl acrylate methochloride, copolymers of acrylamide and dimethylaminoethyl methacrylate methochloride, copolymers of acrylamide and dimethylaminopropyl acrylate methochloride, copolymers of methacrylamide and dimethylaminoethyl methacryl methochloride, copolymers of acrylamide, dimethylaminoethyl acrylate methochloride and acrylic acid, copolymers of acrylamide, dimethylaminoethyl methacrylate methochloride and methacrylic acid and copolymers of acrylamide, dimethylaminoethyl acrylate methochloride and acrylic acid.

The polymers (b) can also be characterized with the help of the K value. They have, for example, a K value of from 15 to 200, preferably 30 to 150 and particularly preferably 45 to 110 (determined in accordance with H. Fikentscher, Cellulose-Chemie, volume 13, 58-64 and 71-74 (1932) in 3% strength by weight aqueous sodium chloride solution at 25° C., a polymer concentration of 0.1% by weight and a pH of 7).

The aqueous dispersions used according to the invention comprise the polymers of group (b), for example, in amounts of from 0.5 to 15% by weight, preferably 1 to 10% by weight. The ratio of the polymers of group (a) to polymers of group (b) in the dispersions used according to the invention is, for example, 1:5 to 5:1 and is preferably in the range from 1:2 to 2:1.

In a preferred embodiment of the invention, the aqueous dispersions of the anionic polymers preferably comprise, as stabilizer, a combination of
(a) at least one graft polymer of vinyl acetate on polyethylene glycols of molecular weight M_{N} from 1000 to 100 000
and
(b1) at least one at least partially hydrolyzed copolymer of vinyl alkyl ether, preferably vinyl methyl ether, and maleic anhydride, which may be present at least partially in saltform.

In a further preferred embodiment of the invention, the following combination of polymers is used:
(a) copolymers of alkylpolyalkylene glycol acrylates or alkylpolyalkylene glycol methacrylates and acrylic acid and/or methacrylic acid
and
(b1) at least one hydrolyzed copolymer of vinyl methyl ether and maleic anhydride in the form of the free carboxyl groups and at least partially in the form of the salts formed with sodium hydroxide solution, potassium hydroxide solution or ammonia.

Further combinations of stabilizers for producing the aqueous dispersions of anionic polymers are, for example, mixtures of
(a) polypropylene glycols, polyethylene glycols and/or block copolymers of ethylene oxide and propylene oxide with molecular weights M_{N} of from 300 to 50 000 and/or polypropylene glycols, polyethylene glycols and/or block copolymers of ethylene oxide and propylene oxide of molecular weight M_{N} from 300 to 50 000 terminally capped at one or both ends with C₁- to C₄-alkyl groups
and
(b2) maltodextrin.

In a further preferred embodiment of the invention, the aqueous dispersions of the anionic polymers preferably comprise, as stabilizer, a combination of
(a) at least one block copolymer of ethylene oxide and propylene oxide
and
(b3) at least one copolymer of methacrylic acid and acrylamidomethylpropanesulfonic acid, copolymer of methyl acrylate, acrylamidomethylpropanesulfonic acid and quaternized vinylimidazole with an overall anionic charge, copolymer of acrylamidomethylpropanesulfonic acid, acrylic acid, methyl acrylate and styrene, polyacrylic acid, polymethacrylic acid and polyacrylamidomethylpropanesulfonic acid.

In a further preferred embodiment of the invention, the aqueous dispersions of the anionic polymers preferably comprise, as stabilizer, a combination of
(a) at least one block copolymer of ethylene oxide and propylene oxide and
(b4) at least one copolymer of acrylamide and dimethylaminoethyl acrylate methochloride.

The copolymer (b4) can, if appropriate, also comprise up to 5 mol % of acrylic acid in copolymerized form.

The weight ratio of components (a) and (b) in the stabilizer mixtures can be varied within a wide range. It can, for example, be 50:1 to 1:10. Preferably, a ratio of (a):(b) of at least 1.5:1, in particular from 7:1 to 10:1 is chosen.

The particle sizes of the anionic W/W polymers in the stable aqueous dispersions are in the range from 0.1 to 200 μm, preferably 0.5 to 70 μm. The particle size can be determined, for example, by optical microscopy, light scattering or freeze-fraction electron microscopy. The aqueous dispersions are prepared, for example, at a pH of from 0.5 to 9, preferably 1 to 5. At a pH below 9, dispersions with a content of anionic polymers of from about 5 to 35% by weight, have a relatively low viscosity. However, if they are diluted to a content of anionic polymers of less than 4% by weight, then the viscosity of the mixture increases considerably.

The anionic W/W emulsion polymers, present if appropriate in the form of an aqueous dispersion, are used as thickeners for cosmetic preparations.

The W/W emulsion polymers present in the form of an aqueous dispersion can be dried in a simple manner to give redispersible polymer powders.

Rheology modifiers and in particular thickeners based on conventional homopolyacrylates are, due to the preparation process (preference is given to precipitation polymerization), usually obtained in solid form, preferably as powders. There is then often the problem of having to convert the solids into a liquid medium again. This is often achieved by gradually wetting the polymer particles with solvent, preferably with water at a pH less than 7, often less than 4, and vigorous and/or long-lasting stirring. Usually, for the preparation of thickened liquid preparations, the conventional thickeners are firstly dissolved in an acidic medium and the other ingredients are added. The incorporation of thickeners based on homopolyacrylates from the prior art into basic media is not possible. The medium thickens immediately after adding the thickeners, and the pulverulent thickener forms insoluble or virtually insoluble particles and it is not possible to establish a defined viscosity in this way.

Upon adding a basic ingredient, for example a neutralizing agent, to the aqueous preparation comprising the anionic polymer, its viscosity increases.

Suitable neutralizing agents are the cosmetically or dermatologically acceptable and customary neutralizing agents. For the neutralization, alkali metal bases, such as sodium hydroxide solution, potassium hydroxide solution, sodium carbonate, sodium hydrogencarbonate, potassium carbonate or potassium hydrogencarbonate and alkaline earth metal bases, such as calcium hydroxide, calcium oxide, magnesium hydroxide or magnesium carbonate, and ammonia or amines can be used. Suitable amines are, for example, C₁-C₆-alkylamines, preferably n-propylamine and n-butylamine, dialkylamines, preferably diethylpropylamine and dipropylmethylamine, trialkylamines, preferably triethylamine and triisopropylamine, C₁-C₆-alkyldiethanolamines, preferably methyl- or ethyldiethanolamine, and di-C₁-C₆-alkylethanolamines. For the neutralization of polymers comprising acid groups, 2-amino-2-methyl-1-propanol (AMP), 2-amino-2-ethylpropane-1,3-diol, diethylaminopropylamine, triisopropanolamine and triethanolamine have proven useful particularly for use in cosmetic preparations, in particular in skin and hair treatment compositions. Neutralization of the polymers comprising acid groups can also be carried out using mixtures of two or more bases, e.g. mixtures of sodium hydroxide solution or potassium hydroxide solution and 2-amino-2-methyl-1-propanol.

The extent of the thickening effect can be determined through the choice of suitable neutralizing agent. Thus, for example, the use of AMP gives rise to a higher viscosity than the use of NaOH.

Further suitable neutralizing agents are disclosed in WO 03/099253, p. 2 I.1 to p. 3, I.6, to the entire scope of which reference is hereby made.

Depending on the intended use, the degree of neutralization can be 5 to 95%, preferably 30 to 95%, or above 99%. In addition, the neutralizing agent can also be added in more than an equivalent amount.

The use of the W/W emulsion polymers gives rise to numerous new possibilities for producing thickened cosmetic preparations. The W/W emulsion polymers can be added to the preparation to be thickened at any pH.

The W/W emulsion polymers and dispersions thereof can be dissolved either in the acidic medium or in the basic medium, advantageously with the application of only small shear forces.

It is particularly advantageous that the W/W emulsion polymers and dispersions thereof can be incorporated into alkaline preparations. Homopolyacrylate thickeners from the prior art can only be incorporated, if at all, only to a very small extent and with the use of high shear forces or long stirring times.

On account of the good solubility of the W/W emulsion polymers and dispersions thereof, only small shear forces are required, and anchor stirrers or paddle stirrers, for example, can be used as stirrers. Complex apparatuses for producing high shear forces are not required. By dispensing with high shear forces, the probability of the polymer chains being degraded is reduced, and thus a lowering of the viscosity is prevented.

One great advantage of the use of the W/W emulsion polymers is that these can be added at any stage of the production of the cosmetic preparations. Thus, the W/W emulsion polymers can, for example, only be added at the end of the production of preparations. This in turn means that the further ingredients can be incorporated into the low viscosity preparation and no high shear forces are required therefor. The incorporation of solid, mechanically labile ingredients is thus made easier.

In contrast to conventional thickeners, for activating the thickening effect of the W/W emulsion polymers, neither high temperatures nor high shear forces or the addition of emulsifiers is required, which considerably simplifies the application.

The polymers suitable for the use according to the invention, and dispersions thereof also have the advantage that, on account of their low viscosity, they are easy to handle and dose and dissolve rapidly in the medium to be thickened. This in turn results in relatively short processing times.

The W/W emulsion polymer dispersion can either be incorporated directly into a cosmetic preparation, for example a hairsetting preparation, preparations for skin or hair cleansing or a shampoo, or a customary drying of the dispersion known to the person skilled in the art is carried out, e.g. spray-drying or freeze-drying, so that the W/W emulsion polymer can be used and processed as powder. For the reasons given above, it is advantageous to incorporate the W/W emulsion polymers in dispersed form.

Using the W/W emulsion polymers, it is possible to prepare gels with a high concentration of high-polarity solvents for all application forms and supply forms according to the invention.

Cosmetic and Dermatological Preparations

The above described emulsion polymers, if appropriate present in the form of aqueous dispersions, are exceptionally suitable according to the invention for use in particular as thickeners in cosmetic preparations.

Such cosmetic preparations are, for example, aqueous, aqueous-alcoholic or alcoholic solutions, O/W, W/O, W/O/W and PIT emulsions, hydrodispersion formulations, solids-stabilized formulations, stick formulations. Important preparation types are creams, foams, sprays (pump spray or aerosol), gels, gel sprays, lotions, oils, oil gels or mousses, which are accordingly formulated with customary further auxiliaries. Preferred cosmetic compositions within the meaning of the present invention are shampoos, gels, gel creams, hydroformulations, stick formulations, cosmetic oils and oil gels, mascara, self-tanning compositions, face care compositions, bodycare compositions, aftersun preparations, hair shaping compositions, hairsetting compositions, hair conditioners and compositions for decorative cosmetics. Cosmetic and dermatological formulations which comprise W/W emulsion polymers become rheologically modified systems as soon as mono- or polyolic components or water are present.

This permits the production of preparations in the form of transparent gels, anhydrous, hydrophilic gels with stabilized water-sensitive active ingredients, the stabilization of oxidation-sensitive substances, the preparation of low-drip or non-drip hair colorants, the preparation of disinfectant gels, the formulation of preservative-free gel systems or the preparation of gels with a high concentration of highly polar solvents such as, for example, glycerol.

This is true for a very wide range of application forms and supply forms in the area of cosmetics and dermatology.

Besides thickening W/W polymers, the cosmetic compositions preferably also comprise cosmetically acceptable additives customary in such formulations, such as emulsifiers and coemulsifiers, solvents, surfactants, oil bodies, preservatives, perfume oils, cosmetic care substances and active ingredients, such as AHA acids, fruit acids, ceramides, phytantriol, collagen, vitamins and provitamins, for example vitamin A, E and C, retinol, bisabolol, panthenol, natural and synthetic photoprotective agents, natural substances, opacifiers, solubility promoters, repellents, bleaches, colorants, tinting agents, tanning agents (e.g. dihydroxyacetone), micropigments, such as titanium oxide or zinc oxide, superfatting agents, pearlescent waxes, consistency regulators, thickeners, solubilizers, complexing agents, fats, waxes, silicone compounds, hydrotropes, dyes, stabilizers, pH regulators, reflectors, proteins and protein hydrolyzates (e.g. wheat, almond or pea proteins), ceramide, protein hydrolyzates, salts, gel formers, consistency regulators, silicones, humectants (e.g. 1,2-pentanediol), refatting agents and further customary additives. Furthermore, other further polymers may be present in particular to establish the properties desired in each case.

It is also advantageous to provide the compositions according to the invention in a liquid form such that cloths of varying material and embossing can be impregnated with them. The person skilled in the art knows how to produce cloths impregnated in this way.

To protect against adverse effects as a result of UV radiation, UV photoprotective agents may also be present in the cosmetic compositions.

The invention further relates to cosmetic compositions comprising the W/W emulsion polymers, if appropriate present in the form of an aqueous dispersion. Particularly preferred cosmetic compositons are shampoos and gels.

The above described W/W emulsion polymers, present if appropriate in the form of an aqueous dispersion, are suitable for producing hair cosmetic preparations such as hair treatments, hair lotions, hair rinses, hair emulsions, end fluids, neutralizers for permanent waves, “hot-oil treatment” preparations, conditioners, setting lotions or hair sprays. Depending on the field of use, the hair cosmetic preparations can be applied as spray, foam, gel, gel spray or mousse.

The W/W emulsion polymers are exceptionally suitable for thickening oxidation hair dyes comprising hydrogen peroxide, and thus for producing viscous low-drip or even non-drip hair colorants.

In addition, on account of their thickening effect, the W/W emulsion polymers can be used in particular in nonaqueous, alcoholic media for stabilizing oxidation-sensitive and/or hydrolysis-sensitive substances such as, for example, vitamin C.

The W/W emulsion polymers are advantageously used for the formulation of subtilisin, lecithin and coenzyme Q10.

Aqueous, alcoholic or aqueous/alcoholic compositions

Preferred compositions are aqueous, alcoholic or aqueous/alcoholic compositions which comprise the at least one W/W polymer in an amount in the range from 0.01 to 20% by weight, particularly preferably from 0.05 to 10% by weight, very particularly preferably from 0.1 to 7% by weight, based on the composition.

Aqueous compositions are understood as meaning compositions which comprise at least 40% by weight, preferably at least 50% by weight and in particular at least 60% by weight, of water and simultaneously less than 20% by weight of alcohol.

Alcoholic compositions are understood as meaning compositions which comprise at least 40% by weight, preferably 50% by weight and in particular at least 60% by weight, of one or more alcohols and simultaneously less than 20% by weight of water.

Aqueous/alcholic compositions are understood as meaning compositions which comprise at least 20% by weight of water and simultaneously at least 20% by weight of alcohol.

A preferred embodiment of the invention are aqueous/alcoholic compositions comprising at least one W/W polymer and preferably at least 50% by weight of water and preferably at most 40% by weight of alcohol.

Another embodiment of the invention is alcoholic compositions comprising at least one W/W polymer and at most 10% by weight, preferably at most 5% by weight, particularly preferably at most 2% by weight and in particular at most 1% by weight of water. Such low-water or virtually anhydrous preparations can be thickened by the W/W polymers.

The W/W polymers suitable for the uses according to the invention are notable for the fact that they can be used as thickeners for preparations whose liquid phase essentially comprises compounds comprising OH groups. These compounds comprising OH groups are essentially water and alcohols.

The W/W polymers suitable for the uses according to the invention are particularly suitable for modifying the rheology of alcoholic preparations. Suitable alcohols for these preparations are generally all alcohols which are present in liquid form at STP. These are, for example, methanol, ethanol, n-propanol, isopropanol, n-butanol, sec-butanol, tert-butanol, 3-methyl-1-butanol (isoamyl alcohol), n-hexanol, cyclohexanol or glycols, such as ethylene glycol, propylene glycol and butylene glycol, polyhydric alcohols such as glycerol, diethylene glycol, triethylene glycol, polyalkylene glycols, such as polyethylene glycol, alkyl ethers of these polyhydric alcohols with number-average molecular weights up to about 3000.

Polyols suitable according to the invention can advantageously be chosen from the group of the at least bifunctional alcohols. In particular, the polyols are advantageously chosen from the following group:

Ethylene glycol, polyethylene glycols with molar masses up to about 2000, propylene glycol-1,2, polypropylene glycols-1,2 with molar masses up to about 2000, propylene glycol-1,3, polypropylene glycols-1,3 with molar masses up to about 2000, butylene glycol-1,2, polybutylene glycols-1,2 with molar masses up to about 2000, butylene glycol-1,3, polybutylene glycols-1,3 with molar masses up to about 2000, butylene glycol-1,4, polybutylene glycols-1,4 with molar masses up to about 2000, butylene glycol-2,3, polybutylene glycols-2,3 with molar masses up to about 2000, glycerol, diglycerol, triglycerol, tetraglycerol and pentaglycerol, where the oligoglycerols are composed of glycerol units condensed via one or more ether bridges, for example as follows:

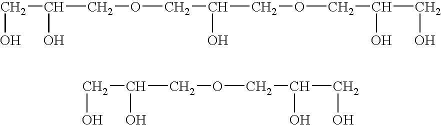

Preference is given to cosmetically acceptable alcohols, in particular the alcohol is or comprises ethanol, glycerol and/or isopropanol, particularly preferably glycerol and/or ethanol.

The W/W polymers act as thickeners both in alcoholic and essentially anhydrous, and also in aqueous and essentially alcohol-free and aqueous/alcoholic compositions.

In a preferred embodiment, the compositions according to the invention comprise
a) 0.05-20% by weight of W/W emulsion polymer
b) 20-99.95% by weight of water and/or alcohol
c) 0-79.5% by weight of further constituents.

Further constituents are understood as meaning the additives customary in cosmetics, for example propellants, antifoams, interface-active compounds, i.e. surfactants, emulsifiers, foam formers and solubilizers. The interface-active compounds used may be anionic, cationic, amphoteric or neutral. Further customary constituents may also be, for example, preservatives, perfume oils, opacifiers, active ingredients, UV filters, care substances such as panthenol, collagen, vitamins, protein hydrolyzates, alpha- and beta-hydroxycarboxylic acids, protein hydrolyzates, stabilizers, pH regulators, dyes, viscosity regulators, gel formers, dyes, salts, humectants, refatting agents and further customary additives.

Hair Cosmetic Preparations

In the hair cosmetic preparations, the W/W polymers can be used in combination with the known styling and conditioner polymers.

Suitable conventional hair cosmetic polymers are, for example, anionic polymers. Such anionic polymers are homopolymers and copolymers of acrylic acid and methacrylic acid or salts thereof, copolymers of acrylic acid and acrylamide and salts thereof; sodium salts of polyhydroxycarboxylic acids, water-soluble or water-dispersible polyesters, polyurethanes (Luviset®P.U.R.) and polyureas. Particularly suitable polymers are copolymers of t-butyl acrylate, ethyl acrylate, methacrylic acid (e.g. Luvimer®100P), copolymers of N-tert-butylacrylamide, ethyl acrylate, acrylic acid (Ultrahold®8, strong), copolymers of vinyl acetate, crotonic acid and, if appropriate, further vinyl esters (e.g. Luviset® grades), maleic anhydride copolymers, if appropriate reacted with alcohols, anionic polysiloxanes, e.g. carboxyfunctional, copolymers of vi nylpyrrolidone, t-butyl acrylate, methacrylic acid (e.g Luviskol®VB M).

Very particularly preferred anionic polymers are acrylates with an acid number greater than or equal to 120 and copolymers of t-butyl acrylate, ethyl acrylate, methacrylic acid.

Further suitable hair cosmetic polymers are cationic polymers with the INCI name Polyquaternium, e.g. copolymers of vinylpyrrolidone/N-vinylimidazolium salts (Luviquat®FC, Luviquat®HM, Luviquat®MS, Luviquat®Care), copolymers of N-vinylpyrrolidone/dimethylaminoethyl methacrylate, quaternized with diethyl sulfate (Luviquat®PQ 11), copolymers of N-vinylcaprolactam N-vinylpyrrolidone/N-vinylimidazolium salts (Luviquat®Hold); cationic cellulose derivatives (Polyquaternium-4 and -10), acrylamide copolymers (Polyquaternium-7).

Suitable further hair cosmetic polymers are also neutral polymers, such as polyvinylpyrrolidones, copolymers of N-vinylpyrrolidone and vinyl acetate and/or vinyl propionate, polysiloxanes, polyvinylcaprolactam and copolymers with N-vinylpyrrolidone, polyethyleneimines and salts thereof, polyvinylamines and salts thereof, cellulose derivatives, polyaspartic acid salts and derivatives.

To establish certain properties, the preparations can additionally also comprise conditioning substances based on silicone compounds. Suitable silicone compounds are, for example, polyalkylsiloxanes, polyarylsiloxanes, polyarylalkylsiloxanes, polyether siloxanes, silicone resins or dimethicone copolyols (CTFA) and aminofunctional silicone compounds such as amodimethicones (CTFA).

The W/W emulsion polymers are suitable, for example, for producing hair styling preparations, in particular clear, transparent gels and gel sprays.

In a preferred embodiment, these preparations comprise
a) 0.1-10% by weight of W/W emulsion polymer
b) 20-99.9% by weight of water and/or alcohol
c) 0-70% by weight of a propellant
d) 0-20% by weight of further constituents

Propellants are the propellants customarily used for hair sprays or aerosol foams. Preference is given to mixtures of propane/butane, pentane, dimethyl ether, 1,1-difluoroethane (HFC-152 a), carbon dioxide, nitrogen or compressed air.

A formulation for aerosol hair foams preferred according to the invention comprises
a) 0.1-10% by weight of W/W emulsion polymer
b) 55-94.8% by weight of water and/or alcohol
c) 5-20% by weight of a propellant
d) 0.1-5% by weight of an emulsifier
e) 0-10% by weight of further constituents

Emulsifiers which can be used are all emulsifiers customarily used in hair foams. Suitable emulsifiers may be nonionic, cationic or anionic.

Examples of nonionic emulsifiers (INCI nomenclature) are laureths, e.g. laureth-4; ceteths, e.g. cetheth-1, polyethylene glycol cetyl ether; ceteareths, e.g. cetheareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methylsulfate, quaternium-1 to x (INCI).

Anionic emulsifiers can be chosen, for example, from the group of alkyl sulfates, alkyl ether sulfates, alkylsulfonates, alkylarylsulfonates, alkylsuccinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

On account of their thickening effect, the W/W emulsion polymers can be used as the sole gel former in the cosmetic preparations. Moreover, they are also suitable for use in combination with other gel formers.

A preparation suitable according to the invention for clear styling gels can, for example, have the following composition:
a) 0.1-10% by weight ofW/Wemulsion polymer
b) 60-99.85% by weight of water and/or alcohol
c) 0-10 % by weight of a further gel former
d) 0-20% by weight of further constituents

Further gel formers which can be used are all gel formers customary in cosmetics. These include slightly crosslinked polyacrylic acid, for example Carbomer (INCI), cellulose derivatives, e.g. hydroxypropylcellulose, hydroxyethylcellulose, cationically modified celluloses, polysaccharides, e.g. xanthum gum, caprylic/capric triglycerides, sodium acrylates copolymer, Polyquaternium-32 (and) Paraffinum Liquidum (INCI), Sodium Acrylates Copolymer (and) Paraffinum Liquidum (and) PPG-1 Trideceth-6, Acrylamidopropyl Trimonium Chloride/Acrylamide Copolymer, Steareth-10 Allyl Ether Acrylates Copolymer, Polyquaternium-37 (and) Paraffinum Liquidum (and) PPG-1 Trideceth-6, Polyquaternium 37 (and) Propylene Glycol Dicaprate Dicaprylate (and) PPG-1 Trideceth-6, Polyquaternium-7, Polyquaternium-44.

One embodiment of the invention is cosmetic preparations, in particular hair gels, on an alcoholic, essentially anhydrous basis with a content of W/W emulsion polymers, at least 30% by weight of C₁-C₄-alcohols and, if appropriate, an alcohol-soluble, film-forming and hair-setting polymer.

Compared to aqueous or aqueous/alcoholic gels, gels based on C₁-C₄-alcohols can satisfy other/complementary requirements for hair gels. If, for example the intention is to produce a setting gel, then it is thus also possible to use alcohol-soluble setting polymers.

The W/W emulsion polymer is preferably used in an amount of from 0.01 to 20% by weight, particularly preferably from 0.05 to 10% by weight, very particularly preferably from 0.1 to 7% by weight. If a hair-setting polymer is used, then preferably in an amount of from 0.1 to 20% by weight, particularly preferably from 0.5 to 15% by weight, very particularly preferably from 1 to 10% by weight. The alcohol is preferably used in an amount of from 50 to 99% by weight, particularly preferably from 70 to 98% by weight. In each case, the % by weight are based on the total weight of the preparation.

In this case, alcohol-soluble polymers are understood as meaning those polymers which are soluble at 25° C. to at least 5% by weight in at least one alcohol having 1 to 4 carbon atoms. Liquid alcohols suitable for the hair gels on an alcoholic, essentially anhydrous basis are mono- or polyhydric alcohols which are liquid at room temperature (20° C.) and have 1 to 4 carbon atoms. These are, in particular, the lower alcohols customarily used for cosmetic purposes, such as, for example, ethanol, isopropanol, glycerol, ethylene glycol or propylene glycol. Particular preference is given to monohydric alcohols having 2 to 4 carbon atoms, in particular ethanol and isopropanol. The hair gel is preferably essentially anhydrous, although, in order to improve the solubility of further ingredients, it can comprise small amounts of water, although the alcohol content significantly exceeds the water content. Essentially anhydrous means that the water content is not greater than 10% by weight, preferably not greater than 5% by weight. The alcoholic gels according to the invention are notable, in the presence of a setting polymer, for good conditioning properties, high degree of setting, rapid drying and pleasant cooling effect.

The preparations according to the invention can be applied to wet or dry hair. The products are suitable both for straight and curly hair.

The W/W emulsion polymers can advantageously also be used in shampoo formulations.

Preferred shampoo formulations comprise
a) 0.05-10% by weight of W/W emulsion polymer
b) 25-94.95% by weight of water
c) 5-50% by weight of surfactant
d) 0-5% by weight of a conditioner
e) 0-5% by weight of a setting agent
f) 0-10% by weight of further cosmetic constituents

In the shampoo formulations, all anionic, neutral, amphoteric or cationic surfactants customarily used in shampoos can be used.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

Of suitability are, for example, sodium lauryl sulfate, ammonium lauryl sulfate, sodium lauryl ether sulfate, ammonium lauryl ether sulfate, sodium lauroyl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or amphopropionates, alkyl amphodiacetates or amphodipropionate.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linea or branched, with ethylene oxide and/or propylene oxide. The amount of alkylene oxide is about 6 to 60 mol per mole of alcohol. Also suitable are alkylamine oxides, mono- or dialkylalkanolamides, fatty acid esters of polyethylene glycols, alkyl polyglycosides or sorbitan ether esters.

Furthermore, the shampoo formulations can comprise customary cationic surfactants, such as, for example, quaternary ammonium compounds, for example cetyltrimethylammonium chloride.

In the shampoo formulations, in order to achieve certain effects, customary conditioners can be used in combination with the emulsion polymers according to the invention. These include, for example, cationic polymers with the INCI name Polyquaternium, in particular copolymers of vinylpyrrolidone/N-vinylimidazolium salts (Luviquat®FC, Luviquat®HM, Luviquat®MS, Luviquat®Care), copolymers of N-vinylpyrrolidone/dimethylaminoethyl methacrylate, quaternized with diethyl sulfate (Luviquat®PQ 11), copolymers of N-vinylcaprolactam/N-vinylpyrrolidone/N-vinyl-imidazolium salts (Luviquat®Hold); cationic cellulose derivatives (Polyquaternium-4 and -10), acrylamide copolymers (Polyquaternium-7). In addition, protein hydrolyzates can be used, as can conditioning substances based on silicone compounds, for example polyalkylsiloxanes, polyarylsiloxanes, polyarylalkylsiloxanes, polyether siloxanes or silicone resins. Further suitable silicone compounds are dimethicone copolyols (CTFA) and aminofunctional silicone compounds such as amodimethicones (CTFA).

Skin Cosmetic Preparations

Skin cosmetic compositions according to the invention, in particular those for caring for the skin, can be present and used in various forms. Thus, for example, they may be an emulsion of the oil-in-water (O/W) type or a multiple emulsion, for example of the water-in-oil-in-water (W/O/W) type. Emulsifier-free formulations such as hydrodispersions, hydrogels or a Pickering emulsion are also advantageous embodiments.

The consistency of the formulations can range from pasty formulations via flowable formulations to thin-liquid, sprayable products. Accordingly, creams, lotions or sprays can be formulated. For use, the cosmetic compositions according to the invention are applied to the skin in an adequate amount in the manner customary for cosmetics and dermatological products.

The salt content of the surface of the skin is sufficient to lower the viscosity of the preparations according to the invention in such a way as to enable simple distribution and incorporation of the preparations.

The skin cosmetic preparations according to the invention are present in particular as W/O or O/W skin creams, day and night creams, eye creams, face creams, antiwrinkle creams, mimic creams, moisturizing creams, bleach creams, vitamin creams, skin lotions, care lotions and moisturizing lotions.

Further advantageous skin cosmetic preparations are face tonics, face masks, deodorants and other cosmetic lotions and preparations for decorative cosmetics, for example concealing sticks, stage make-up, mascara, eye shadows, lipsticks, kohl pencils, eyeliners, makeup, foundations, blushers, powder and eyebrow pencils.

Furthermore, the compositions according to the invention can be used in nose strips for pore cleansing, in antiacne compositions, repellents, shaving compositions, hair-removal compositions, intimate care compositions, foot care compositions, and in baby care.

Besides the W/W emulsion polymer and suitable carriers, the skin cosmetic preparations according to the invention also comprise further active ingredients and/or auxiliaries customary in cosmetics, as described above and below.

These include, preferably, emulsifiers, preservatives, perfume oils, cosmetic active ingredients such as phytantriol, vitamin A, E and C, retinol, bisabolol, panthenol, natural and synthetic photoprotective agents, bleaches, colorants, tinting agents, tanning agents, collagen, protein hydrolyzates, stabilizers, pH regulators, dyes, salts, thickeners, gel formers, consistency regulators, silicones, humectants, conditioners, refatting agents and further customary additives.

Further polymers can also be added to the compositions if specific properties are to be established. To establish certain properties, for example, improving the feel to the touch, the spreading behavior, the water resistance and/or the binding of active ingredients and auxiliaries, such as pigments, the compositions can additionally also comprise conditioning substances based on silicone compounds. Suitable silicone compounds are, for example, polyalkylsiloxanes, polyarylsiloxanes, polyarylalkylsiloxanes, polyether siloxanes or silicone resins.

Further possible ingredients of the compositions according to the invention are described below under the respective key word.

Oils, Fats and Waxes

The skin and hair cosmetic compositions also preferably comprise oils, fats or waxes.

Constituents of the oil and/or fat phase of the cosmetic compositions are advantageously chosen from the group of lecithins and fatty acid triglycerides, namely the triglycerol esters of saturated and/or unsaturated, branched and/or unbranched alkanecarboxylic acids with a chain length of from 8 to 24, in particular 12 to 18, carbon atoms. The fatty acid triglycerides can, for example, be chosen advantageously from the group of synthetic, semisynthetic and natural oils, such as, for example, olive oil, sunflower oil, soybean oil, peanut oil, rapeseed oil, almond oil, palm oil, coconut oil, castor oil, wheat germ oil, grape seed oil, thistle oil, evening primrose oil, macadamia nut oil and the like. Further polar oil components can be chosen from the group of esters of saturated and/or unsaturated, branched and/or unbranched alkanecarboxylic acids with a chain length of from 3 to 30 carbon atoms and saturated and/or unsaturated, branched and/or unbranched alcohols with a chain length of from 3 to 30 carbon atoms, and also from the group of esters of aromatic carboxylic acids and saturated and/or unsaturated, branched and/or unbranched alcohols with a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, dicaprylyl carbonate (cetiol CC) and cocoglycerides (Myritol 331), butylene glycol dicaprylate/dicaprate and dibutyl adipate, and synthetic, semisynthetic and natural mixtures of such esters, such as, for example, jojoba oil.

In addition, one or more oil components can be chosen advantageously from the group of branched and unbranched hydrocarbons and hydrocarbon waxes, silicone oils, dialkyl ethers, the group of saturated or unsaturated, branched or unbranched alcohols.

Any mixtures of such oil and wax components can also be used advantageously for the purposes of the present invention. It may also, if appropriate, be advantageous to use waxes, for example cetyl palmitate, as the sole lipid component of the oil phase.

According to the invention, the oil component is chosen advantageously from the group consisting of 2-ethylhexyl isostearate, octyldodecanol, isotridecyl isononanoate, isoeicosane, 2-ethylhexyl cocoate, C12-15-alkyl benzoate, caprylic/capric triglyceride, dicaprylyl ether.

Of advantage according to the invention are mixtures of C12-15-alkyl benzoate and 2-ethylhexyl isostearate, mixtures of C12-15-alkyl benzoate and isotridecyl isononanoate, and mixtures of C12-15-alkyl benzoate, 2-ethylhexyl isostearate and isotridecyl isononanoate.

According to the invention, as oils with a polarity of from 5 to 50 mN/m, particular preference is given to using fatty acid triglycerides, in particular soybean oil and/or almond oil.

Of the hydrocarbons, paraffin oil, squalane, squalene and in particular polyisobutenes, which may also be hydrogenated, are to be used for the purposes of the present invention.

In addition, the oil phase can be chosen advantageously from the group of Guerbet alcohols. They are formed in accordance with the reaction equation

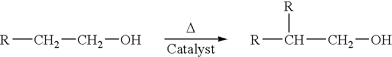

by oxidation of an alcohol to an aldehyde, by aldol condensation of the aldehyde, elimination of water from the aldol and hydrogenation of the allyl aldehyde. Guerbet alcohols are even liquid at low temperatures and bring about virtually no skin irritations. They can be used advantageously as fatting, superfatting and also refatting constituents in cosmetic compositions.

The use of Guerbet alcohols in cosmetics is known per se. Such species are then characterized in most cases by the structure

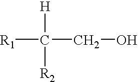

Here, R₁ and R₂ are generally unbranched alkyl radicals.

According to the invention, the Guerbet alcohol(s) is/are advantageously chosen from the group where

R₁=propyl, butyl, pentyl, hexyl, heptyl or octyl and

R₂=hexyl, heptyl, octyl, nonyl, decyl, undecyl, dodecyl, tridecyl or tetradecyl.

Guerbet alcohols preferred according to the invention are 2-butyloctanol (commercially available, for example, as Isofol®12 (Condea)) and 2-hexyldecanol (commercially available, for example, as Isofol®16 (Condea)).

Mixtures of Guerbet alcohols according to the invention are also to be used advantageously according to the invention, such as, for example, mixtures of 2-butyl-octanol and 2-hexyldecanol (commercially available, for example, as Isofol®14 (Condea)).

Any mixtures of such oil and wax components are also to be used advantageously for the purposes of the present invention. Among the polyolefins, polydecenes are the preferred substances.

The oil component can advantageously also have a content of cyclic or linear silicone oils or consist entirely of such oils, although it is preferred to use an additional content of other oil phase components apart from the silicone oil or the silicone oils.

Low molecular weight silicones or silicone oils are generally defined by the following general formula

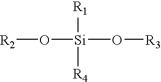

Higher molecular weight silicones or silicone oils are generally defined by the following general formula

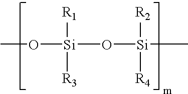

where the silicon atoms may be substituted by identical or different alkyl radicals and/or aryl radicals, which are depicted here in general terms by the radicals R₁ to R₄. The number of different radicals is not, however, necessarily limited to 4. m can assume values from 2 to 200 000.

Cyclic silicones to be used advantageously according to the invention are generally defined by the following general formula

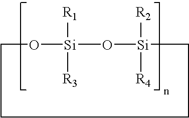

where the silicon atoms can be substituted by identical or different alkyl radicals and/or aryl radicals, which are depicted here in general terms by the radicals R₁ to R₄. The number of different radicals is, however, not necessarily limited to 4. n here can assume values from 3/2 to 20. Fractional values for n take into account that uneven numbers of siloxyl groups may be present in the cycle.

Phenyltrimethicone is advantageously chosen as silicone oil. Other silicone oils, for example dimethicone, hexamethylcyclotrisiloxane, phenyldimethicone, cyclomethicone (e.g. decamethylcyclopentasiloxane), hexamethylcyclotrisiloxane, polydimethylsiloxane, poly(methylphenylsiloxane), cetyldimethicone, behenoxydimethicone are also to be used advantageously for the purposes of the present invention. Also advantageous are mixtures of cyclomethicone and isotridecyl isononanoate, and those of cyclomethicone and 2-ethylhexyl isostearate.

It is, however, also advantageous to choose silicone oils of similar constitution to the compounds described above whose organic side chains are derivatized, for example polyethoxylated and/or polypropoxylated. These include, for example, polysiloxane polyalkyl-polyether copolymers, such as, for example, cetyldimethicone copolyol.

Cyclomethicone (octamethylcyclotetrasiloxane) is used advantageously as silicone oil to be used according to the invention.

Fat and/or wax components to be used advantageously can be chosen from the group of vegetable waxes, animal waxes, mineral waxes and petrochemical waxes. For example, candelilla wax, carnauba wax, Japan wax, esparto grass wax, cork wax, guaruma wax, rice germ oil wax, sugar cane wax, berry wax, ouricury wax, montan wax, jojoba wax, shea butter, beeswax, shellac wax, spermaceti, lanolin (wool wax), uropygial grease, ceresin, ozokerite (earth wax), paraffin waxes and microwaxes are advantageous.

Further advantageous fat and/or wax components are chemically modified waxes and synthetic waxes, such as, for example, Syncrowax®HRC (glyceryl tribehenate), and Syncrowax®AW 1 C (C₁₈₋₃₆-fatty acid), and montan ester waxes, sasol waxes, hydrogenated jojoba waxes, synthetic or modified beeswaxes (e.g. dimethicone copolyol beeswax and/or C₃₀₋₅₀-alkyl beeswax), cetyl ricinoleates, such as, for example, Tegosoft®CR, polyalkylene waxes, polyethylene glycol waxes, but also chemically modified fats, such as, for example, hydrogenated vegetable oils (for example hydrogenated castor oil and/or hydrogenated coconut fatty glycerides), triglycerides, such as, for example, hydrogenated soy glyceride, trihydroxystearin, fatty acids, fatty acid esters and glycol esters, such as, for example, C₂₀₋₄₀-alkyl stearate, C₂₀₋₄₀-alkyl hydroxystearoylstearate and/or glycol montanate. Further advantageous are also certain organosilicon compounds which have similar physical properties to the specified fat and/or wax components, such as, for example, stearoxytrimethylsilane.

According to the invention, the fat and/or wax components can be used either individually or as a mixture in the compositions.

Any mixtures of such oil and wax components are also to be used advantageously for the purposes of the present invention.

The oil phase is advantageously chosen from the group consisting of 2-ethylhexyl isostearate, octyldodecanol, isotridecyl isononanoate, butylene glycol dicaprylate/dicaprate, 2-ethylhexyl cocoate, C₁₂₋₁₅-alkylbenzoate, caprylic/capric triglyceride, dicaprylyl ether.

Of particular advantage are mixtures of octyldodecanol, caprylic/capric triglyceride, dicaprylyl ether, dicaprylyl carbonate, cocoglycerides or mixtures of C₁₂₋₁₅-alkyl benzoate and 2-ethylhexyl isostearate, mixtures of C₁₂₋₁₅-alkyl benzoate and butylene glycol dicaprylate/dicaprate, and mixtures of C₁₂₋₁₅-alkyl benzoate, 2-ethylhexyl isostearate and isotridecyl isononanoate.

Of the hydrocarbons, paraffin oil, cycloparaffin, squalane, squalene, hydrogenated polyisobutene and polydecene are to be used advantageously for the purposes of the present invention.

The oil component can also advantageously be chosen from the group of phospholipids. The phospholipids are phosphoric esters of acylated glycerols. Of greatest importance among the phosphatidylcholines are, for example, the lecithins, which are characterized by the general structure

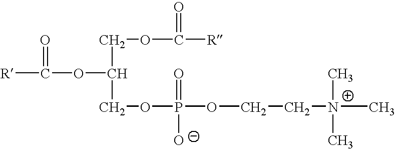

where R′ und R″ are typically unbranched aliphatic radicals having 15 or 17 carbon atoms and up to 4 cis double bonds.

Paraffin oil advantageous according to the invention which can be used in accordance with the invention is Merkur Weissoel Pharma 40 from Merkur Vaseline, Shell Ondina® 917, Shell Ondina® 927, Shell Oil 4222, Shell Ondina®933 from Shell & DEA Oil, Pionier® 6301 S. Pionier® 2071 (Hansen & Rosenthal).

Suitable cosmetically compatible oil and fat components are described in Karl-Heinz Schrader, Grundlagen und Rezepturen der Kosmetika [Fundamentals and formulations of cosmetics], 2nd edition, Verlag Huthig, Heidelberg, pp. 319-355, which is hereby incorporated in its entirety by reference.

Conditioners

In a preferred embodiment, the cosmetic compositions also comprise conditioners.

Suitable conditioners are, for example, those compounds which are listed in the International Cosmetic Ingredient Dictionary and Handbook (Volume 4, editor: R. C. Pepe, J. A. Wenninger, G. N. McEwen, The Cosmetic, Toiletry, and Fragrance Association, 9th edition, 2002) under Section 4 under the keywords “Hair Conditioning Agents”, “Humectants”, “Skin-Conditioning Agents”, “Skin-Conditioning Agents-Emollient”, “Skin-Conditioning Agents-Humectant”, “Skin-Conditioning Agents-Miscellaneous”, “Skin-Conditioning Agents-Occlusive” and “Skin Protectants”, and all of the compounds listed in EP-A 934 956 (pp.11-13) under “water soluble conditioning agent” and “oil soluble conditioning agent”. Further advantageous conditioning substances are represented by, for example, the compounds referred to according to INCI as Polyquaternium (in particular Polyquaternium-1 to Polyquaternium-56).

Suitable conditioners include, for example, also polymeric quaternary ammonium compounds, cationic cellulose derivatives, chitosan derivatives and polysaccharides.

Conditioners advantageous according to the invention can be chosen here also from the compounds shown in Table 1 below.

TABLE 1
Conditioners to be used advantageously

| | | | Example |
|---|---|---|---|
| INCI name | CAS number | Polymer type | (trade name) |
| Polyquaternium-2 | CAS 63451-27-4 | Urea, N,N′-bis[3-(dimethyl- | Mirapol ® A-15 |
| | | amino)propyl] polymer with | |
| | | 1,1′-oxybis(2-chloroethane) | |
| Polyquaternium-5 | CAS 26006-22-4 | Acrylamide, β-methacryloxy- | |
| | | ethyltriethylammonium metho- | |
| | | sulfate | |
| Polyquaternium-6 | CAS 26062-79-3 | N,N-Dimethyl-N-2-propenyl- | Merquat ® 100 |
| | | 2-propenaminium chloride | |
| Polyquaternium-7 | CAS 26590-05-6 | N,N-Dimethyl-N-2-propenyl- | Merquat ® S |
| | | 2-propenaminium chloride, | |
| | | 2-propenamide | |
| Polyquaternium-10 | CAS 53568-66-4, | Quaternary ammonium salt of | Celquat ® SC-230M, |
| | 55353-19-0, 54351-50-7 | hydroxyethylcellulose | Polymer JR 400 |
| | 68610-92-4, 81859-24-7 | | |
| Polyquaternium-11 | CAS 53633-54-8 | Vinylpyrrolidone/dimethylamino- | Gafquat ® 755N |
| | | ethyl methacrylate | |
| | | copolymer/diethyl sulfate | |
| | | reaction product | |
| Polyquaternium-16 | CAS 29297-55-0 | Vinylpyrrolidone/- | Luviquat ® HM552 |
| | | vinylimidazolinum methochloride | |
| | | copolymer | |
| Polyquaternium-17 | CAS 90624-75-2 | | Mirapol ® AD-1 |
| Polyquaternium-19 | CAS 110736-85-1 | Quaternized water-soluble | |
| | | polyvinyl alcohol | |
| Polyquaternium-20 | CAS 110736-86-2 | Quaternized polyvinyl octadecyl | |
| | | ether dispersible in water | |
| Polyquaternium-21 | | Polysiloxane-polydimethyl- | Abil ® B 9905 |
| | | dimethylammonium acetate | |
| | | copolymer | |
| Polyquaternium-22 | CAS 53694-17-0 | Dimethyldiallylammonium | Merquat ® 280 |
| | | chloride/acrylic acid copolymer | |
| Polyquaternium-24 | CAS 107987-23-5 | Polymeric quaternary | Quartisoft ® LM-200 |
| | | ammonium salt of | |
| | | hydroxyethylcellulose | |
| Polyquaternium-28 | CAS 131954-48-8 | Vinylpyrrolidone/methacrylamido- | Gafquat ® HS-100 |
| | | propyltrimethylammonium | |
| | | chloride copolymer | |
| Polyquaternium-29 | CAS 92091-36-6, | Chitosan which has been | Lexquat ® CH |
| | 148800-30-2 | reacted with propylene oxide | |
| | | and quaternized with | |
| | | epichlorohydrin | |
| Polyquaternium-31 | CAS 136505-02-7, | Polymeric quaternary | Hypan ® QT 100 |
| | 139767-67-7 | ammonium salt which is | |
| | | produced via the reaction of | |
| | | DMAPA-acrylate/acrylic | |
| | | acid/acrylonitrogen copolymer | |
| | | and diethyl sulfate | |
| Polyquaternium-32 | CAS 35429-19-7 | N,N,N-Trimethyl-2-([82-methyl- | |
| | | 1-oxo-2-propenyl)oxy]- | |
| | | ethanaminium chloride, polymer | |
| | | with 2-propenamide | |
| Polyquaternium-37 | CAS 26161-33-1 | | |
| Polyquaternium-44 | | Copolymeric quaternary | |
| | | ammonium salt of vinyl- | |
| | | pyrrolidone and quaternized | |
| | | imidazoline | |

Further conditioners advantageous according to the invention are cellulose derivatives and quaternized guar gum derivatives, in particular guar hydroxypropylammonium chloride (e.g. Jaguar Excel®, Jaguar C 162® (Rhodia), CAS 65497-29-2, CAS 39421-75-5). Nonionic poly-N-vinylpyrrolidone/polyvinyl acetate copolymers (e.g. Luviskol®VA 64 (BASF)), anionic acrylate copolymers (e.g. Luviflex® Soft (BASF)), and/or amphoteric amide/acrylate/methacrylate copolymers (e.g. Amphomer® (National Starch)) can also be used advantageously as conditioners according to the invention. Further possible conditioners are quaternized silicones.

Thickeners

Apart from the W/W emulsion polymers, the cosmetic compositions according to the invention can also comprise further thickeners. Suitable thickeners are crosslinked polyacrylic acids and derivatives thereof, polysaccharides, such as xanthan gum, guar guar, agar agar, alginates or tyloses, cellulose derivatives, e.g. carboxymethylcellulose or hydroxycarboxymethylcellulose, also higher molecular weight polyethylene glycol mono- and diesters of fatty acids, fatty alcohols, monoglycerides and fatty acids, polyvinyl alcohol and polyvinylpyrrolidone.

Suitable thickeners are also polyacrylates, such as Carbopol® (Noveon), Ultrez® (Noveon), Luvigel® EM (BASF), Capigel®98 (Seppic), Synthalene® (Sigma), the Aculyn® grades from Rohm und Haas, such as Aculyn® 22 (copolymer of acrylates and methacrylic acid ethoxides with stearyl radical (20 EO units)) and Aculyn® 28 (copolymer of acrylates and methacrylic acid ethoxylates with behenyl radical (25 EO units)).

Suitable thickeners are also, for example, aerosil grades (hydrophilic silicas), polyacrylamides, polvinyl alcohol and polyvinylpyrrolidone, surfactants, such as, for example, ethoxylated fatty acid glyercides, esters of fatty acids with polyols, such as, for example, pentaerythritol or trimethylolpropane, fatty alcohol ethoxylates with narrowed homolog distribution or alkyl oligoglucosides, and electrolytes, such as sodium chloride and ammonium chloride. Particularly preferred thickeners for the preparation of gels are Ultrez®21, Aculyn®28, Luvigel® EM and Capigel®98.

Particularly in the case of more highly concentrated compositions, it is also possible, to regulate the consistency, to also add substances which reduce the viscosity of the formulation, such as, for example, propylene glycol or glycerol. These substances influence the product properties only slightly.

In a preferred embodiment of the invention, the cosmetic preparations comprise no further thickeners apart from the W/W emulsion polymers.

Preservatives

The aqueous cosmetic compositions according to the invention can also comprise preservatives. Compositions with high water contents have to be reliably protected against the build-up of germs. The most important preservatives used for this purpose are urea condensates, p-hydroxybenzoic esters, the combination of phenoxyethanol with methyldibromoglutaronitrile and acid preservatives with benzoic acid, salicylic acid and sorbic acid.

Compositions with high fractions of surfactants or polyols and low water contents can also be formulated without preservatives.

The compositions according to the invention can comprise one or more preservatives. Advantageous preservatives for the purposes of the present invention are, for example, formaldehyde donors (such as, for example, DMDM hydantoin, which is commercially available, for example, under the trade name Glydant® (Lonza)), iodopropyl butylcarbamates (e.g. Glycacil-L®, Glycacil-S® (Lonza), Dekaben®LMB (Jan Dekker)), parabens (p-hydroxybenzoic alkyl esters, such as, for example, methyl-, ethyl-, propyl- and/or butylparaben), dehydroacetic acid (Euxyl® K 702 (Schülke&Mayr), phenoxyethanol, ethanol, benzoic acid. So-called preservative aids, such as, for example, octoxyglycerol, glycine, soybean etc. are also used advantageously.

The table below gives an overview of customary preservatives which may also be present in the cosmetic compositions according to the invention.

| | |
|---|---|
| E 200 | Sorbic acid |
| E 201 | Sodium sorbate |
| E 202 | Potassium sorbate |
| E 203 | Calcium sorbate |
| E 210 | Benzoic acid |
| E 211 | Sodium benzoate |
| E 212 | Potassium benzoate |
| E 213 | Calcium benzoate |
| E 214 | Ethyl p-hydroxybenzoate |
| E 215 | Ethyl p-hydroxybenzoate Na salt |
| E 216 | n-Propyl p-hydroxybenzoate |
| E 217 | n-Propyl p-hydroxybenzoate Na salt |
| E 218 | Methyl p-hydroxybenzoate |
| E 219 | Methyl p-hydroxybenzoate Na salt |
| E 220 | Sulfur dioxide |
| E 221 | Sodium sulfite |
| E 222 | Sodium hydrogensulfite |
| E 223 | Sodium disulfite |
| E 224 | Potassium disulfite |
| E 226 | Calcium sulfite |
| E 227 | Calcium hydrogensulfite |
| E 228 | Potassium hydrogensulfite |
| E 230 | Biphenyl (diphenyl) |
| E 231 | Orthophenylphenol |
| E 232 | Sodium orthophenyl phenoxide |
| E 233 | Thiabendazole |
| E 235 | Natamycin |
| E 236 | Formic acid |
| E 237 | Sodium formate |
| E 238 | Calcium formate |
| E 239 | Hexamethylenetetramine |
| E 249 | Potassium nitrite |
| E 250 | Sodium nitrite |
| E 251 | Sodium nitrate |
| E 252 | Potassium nitrate |
| E 280 | Propionic acid |
| E 281 | Sodium propionate |
| E 282 | Calcium propionate |
| E 283 | Potassium propionate |
| E 290 | Carbon dioxide |

Also advantageous are preservatives or preservative aids customary in cosmetics, such as dibromodicyanobutane (2-bromo-2-bromomethylglutarodinitrile), phenoxyethanol, 3-iodo-2-propynyl butylcarbamate, 2-bromo-2-nitropropane-1,3-diol, imidazolidinylurea, 5-chloro-2-methyl-4-isothiazolin-3-one, 2-chloroacetamide, benzalkonium chloride, benzyl alcohol, salicylic acid and salicylates.

It is particularly preferred if the preservatives used are iodopropyl butylcarbamates, parabens (methyl, ethyl, propyl and/or butyl paraben) and/or phenoxyethanol.

For stabilizing alcoholic, in particular ethanolic, compositions, no preservatives are often necessary. Consequently, preferred cosmetic preparations based on alcohol or water/alcohol which comprise a W/W emulsion polymer as thickener require no preservatives.

Moreover, the W/W emulsion polymers are exceptionally suitable for producing disinfection gels based on alcohol (ethanol, isopropanol, polyethylene glycols which are liquid at STP, such as, for example, PEG-8).

Complexing Agents

Since the raw materials and also the compositions themselves are prepared predominantly in steel apparatuses, the end products can comprise iron (ions) in trace amounts. In order to prevent these impurities adversely affecting the product quality via reactions with dyes and perfume oil constituents, complexing agents such as salts of ethylenediaminetetraacetic acid, of nitrilotriacetic acid, of iminodisuccinic acid or phosphates, are added.

UV Photoprotective Filters

In order to stabilize the ingredients present in the compositions according to the invention, such as, for example, dyes and perfume oils, against changes due to UV light, it is possible to incorporate UV photoprotective filters, such as, for example, benzophenone derivatives. Of suitability for this purpose are all cosmetically acceptable UV photoprotective filters.

Examples of UV photoprotective filters which may be present in the compositions according to the invention are:

| | | CAS No. |
|---|---|---|
| No. | Substance | (=acid) |
| 1 | 4-Aminobenzoic acid | 150-13-0 |
| 2 | 3-(4′-Trimethylammonium)benzylidenebornan-2-one methyl- | 52793-97-2 |
| | sulfate | |
| 3 | 3,3,5-Trimethylcyclohexyl salicylate | 118-56-9 |
| | (homosalate) | |
| 4 | 2-Hydroxy-4-methoxybenzophenone | 131-57-7 |
| | (oxybenzone) | |
| 5 | 2-Phenylbenzimidazole-5-sulfonic acid and its potassium, sodium | 27503-81-7 |
| | and triethanolamine salts | |
| 6 | 3,3′-(1,4-Phenylenedimethine)bis(7,7-dimethyl- | 90457-82-2 |
| | 2-oxobicyclo[2.2.1]heptane-1-methanesulfonic acid) and its | |
| | salts | |
| 7 | Polyethoxyethyl 4-bis(polyethoxy)aminobenzoate | 113010-52-9 |
| 8 | 2-Ethylhexyl 4-dimethylaminobenzoate | 21245-02-3 |
| 9 | 2-Ethylhexyl salicylate | 118-60-5 |
| 10 | 2-Isoamyl 4-methoxycinnamate | 71617-10-2 |
| 11 | 2-Ethylhexyl 4-methoxycinnamate | 5466-77-3 |
| 12 | 2-Hydroxy-4-methoxybenzophenone-5-sulfonic acid (sulisobenzone) | 4065-45-6 |
| | and the sodium salt | |
| 13 | 3-(4′-Sulfobenzylidene)bornan-2-one and salts | 58030-58-6 |
| 14 | 3-Benzylidenebornan-2-one | 16087-24-8 |
| 15 | 1-(4′-Isopropylphenyl)-3-phenylpropane-1,3-dione | 63260-25-9 |
| 16 | 4-Isopropylbenzyl salicylate | 94134-93-7 |
| 17 | 3-Imidazol-4-ylacrylic acid and its ethyl ester | 104-98-3 |
| 18 | Ethyl 2-cyano-3,3-diphenylacrylate | 5232-99-5 |
| 19 | 2′-Ethylhexyl 2-cyano-3,3-diphenylacrylate | 6197-30-4 |
| 20 | Menthyl o-aminobenzoate or: | 134-09-8 |
| | 5-methyl-2-(1-methylethyl)-2-aminobenzoate | |
| 21 | Glyceryl p-aminobenzoate or: | 136-44-7 |
| | 1-glyceryl 4-aminobenzoate | |
| 22 | 2,2′-Dihydroxy-4-methoxybenzophenone (dioxybenzone) | 131-53-3 |
| 23 | 2-Hydroxy-4-methoxy-4-methylbenzophenone (mexenone) | 1641-17-4 |
| 24 | Triethanolamine salicylate | 2174-16-5 |
| 25 | Dimethoxyphenylglyoxalic acid or: | 4732-70-1 |
| | 3,4-dimethoxyphenylglyoxal acidic sodium | |
| 26 | 3-(4′-Sulfobenzylidene)bornan-2-one and its salts | 56039-58-8 |
| 27 | 4-tert-Butyl-4′-methoxydibenzoylmethane | 70356-09-1 |
| 28 | 2,2′,4,4′-Tetrahydroxybenzophenone | 131-55-5 |
| 29 | 2,2′-Methylenebis[6-(2H-benzotriazol-2-yl)- | 103597-45-1 |
| | 4-(1,1,3,3,-tetramethylbutyl)phenol] | |
| 30 | 2,2′-(1,4-Phenylene)bis-1H-benzimidazole-4,6-disulfonic acid, | 180898-37-7 |
| | Na salt | |
| 31 | 2,4-bis[4-(2-Ethylhexyloxy)-2-hydroxy]phenyl- | 187393-00-6 |
| | 6-(4-methoxyphenyl)(1,3,5)-triazine | |
| 32 | 3-(4-Methylbenzylidene)camphor | 36861-47-9 |
| 33 | Polyethoxyethyl 4-bis(polyethoxy)paraaminobenzoate | 113010-52-9 |
| 34 | 2,4-Dihydroxybenzophenone | 131-56-6 |
| 35 | 2,2′-Dihydroxy-4,4′-dimethoxybenzophenone-5,5′-disodium sulfonate | 3121-60-6 |
| 36 | Benzoic acid, 2-[4-(diethylamino)-2-hydroxybenzoyl], hexyl ester | 302776-68-7 |
| 37 | 2-(2H-Benzotriazol-2-yl)-4-methyl-6-[2-methyl-3-[1,3,3,3- | 155633-54-8 |
| | tetramethyl-1-[(trimethylsilyl)oxy]disiloxanyl]propyl]phenol | |
| 38 | 1,1-[(2,2′-Dimethylpropoxy)carbonyl]-4,4-diphenyl-1,3-butadiene | 363602-15-7 |

Photoprotective agents suitable for use in the compositions according to the invention are also the compounds specified in EP-A 1 084 696 in paragraphs [0036] to [0053], which is hereby incorporated in its entirety at this point by reference. Of suitability for the use according to the invention are all UV photoprotective filters which are specified in Annex 7 (to §3b) of the German Cosmetics Directive under “Ultraviolet filters for cosmetic compositions”.

The list of specified UV photoprotective filters which can be used in the compositions according to the invention is not exhaustive.

Antioxidants

An additional content of antioxidants is generally preferred. According to the invention, antioxidants which can be used are all antioxidants which are customary or suitable for cosmetic applications. The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g anserine), carotenoids, carotenes (e.g. α-carotene, β-carotene, γ-lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, n-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, furfurylidenesorbitol and derivatives thereof, ubiquinone and ubiquinol and derivatives thereof, vitamin C and derivatives (e.g. ascorbyl palmitate, Mg-ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄) selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) suitable according to the invention of these specified active ingredients.

The amount of the abovementioned antioxidants (one or more compounds) in the compositions is preferably 0.001 to 30% by weight, particularly preferably 0.05 to 20% by weight, in particular 0.1 to 10% by weight, based on the total weight of the composition.

If vitamin E and/or derivatives thereof are the antioxidant or antioxidants, it is advantageous to prepare these in concentrations of from 0.001 to 10% by weight, based on the total weight of the composition.

If vitamin A, or vitamin A derivatives, or carotenes or derivatives thereof are the antioxidant or the antioxidants, it is advantageous to prepare these in concentrations of from 0.001 to 10% by weight, based on the total weight of the composition.

Buffers

Buffers ensure the pH stability of aqueous compositions according to the invention. Preference is given to using citrate, lactate and phosphate buffers.

Solubility Promoters

These are used in order to form clear solutions of care oils or perfume oils and also to keep them in clear solution at low temperatures. The most common solubility promoters are ethoxylated nonionic surfactants, e.g. hydrogenated and ethoxylated castor oils.

Antimicrobial Agents

In addition, it is also possible to use antimicrobial agents. These include generally all suitable preservatives with a specific effect against Gram-positive bacteria, e.g. triclosan (2,4,4′-trichloro-2′-hydroxydiphenyl ether), chlorhexidine (1,1′-hexamethylenebis[5-(4-chlorophenyl)biguanide) and TTC (3,4,4′-trichlorocarbanilide). Quaternary ammonium compounds are in principle likewise suitable. Numerous fragrances also have antimicrobial properties. A large number of essential oils and their characteristic ingredients, such as, for example, oil of cloves (eugenol), mint oil (menthol) or thyme oil (thymol) also exhibit a marked antimicrobial effectiveness.

The antibacterially effective substances are generally used in concentrations of from about 0.1 to 0.3% by weight.

Dispersants

If it is the aim to disperse insoluble active ingredients, such as antidandruff active ingredients or silicone oils, in the composition and to keep them permanently in suspension, it is advantageous to use dispersants and thickeners, such as, for example, magnesium aluminum silicates, bentonites, fatty acyl derivatives, polyvinylpyrrolidone or hydrocolloids, e.g. xanthan gum or carbomers.

Apart from the abovementioned substances, the compositions in accordance with the invention comprise, if appropriate, the further additives customary in cosmetics, for example perfume, dyes, antimicrobial substances, refatting agents, complexing agents and sequestering agents, pearlescent agents, plant extracts, vitamins, active ingredients, bactericides, pigments which have a coloring effect, softening, moisturizing and/or humectant substances, or other customary constituents of a cosmetic or dermatological formulation, such as alcohols, polyols, polymers, organic acids for adjusting the pH, foam stabilizers, electrolytes, organic solvents or silicone derivatives.

Ethoxylated Glycerol Fatty Acid Esters

The cosmetic compositions according to the invention comprise, if appropriate, ethoxylated oils chosen from the group of ethoxylated glycerol fatty acid esters, particularly preferably PEG-10 olive oil glycerides, PEG-11 avocado oil glycerides, PEG-11 cocoa butter glycerides, PEG-13 sunflower oil glycerides, PEG-15 glyceryl isostearate, PEG-9 coconut fatty acid glycerides, PEG-54 hydrogenated castor oil, PEG-7 hydrogenated castor oil, PEG-60 hydrogenated castor oil, jojoba oil ethoxylate (PEG-26 jojoba fatty acids, PEG-26 jojoba alcohol), glycereth-5 cocoate, PEG-9 coconut fatty acid glycerides, PEG-7 glyceryl cocoate, PEG-45 palm kernel oil glycerides, PEG-35 castor oil, olive oil PEG-7 ester, PEG-6 caprylic/capric glycerides, PEG-10 olive oil glycerides, PEG-13 sunflower oil glycerides, PEG-7 hydrogenated castor oil, hydrogenated palm kernel oil glyceride PEG-6 ester, PEG-20 corn oil glycerides, PEG-18 glycerol oleate cocoate, PEG-40 hydrogenated castor oil, PEG-40 castor oil, PEG-60 hydrogenated castor oil, PEG-60 corn oil glycerides, PEG-54 hydrogenated castor oil, PEG-45 palm kernel oil glycerides, PEG-80 glyceryl cocoate, PEG-60 almond oil glycerides, PEG-60 Evening Primrose glyceride, PEG-200 hydrogenated glyceryl palmate, PEG-90 glyceryl isostearate.

Preferred ethoxylated oils are PEG-7 glyceryl cocoate, PEG-9 cocoglycerides, PEG-40 hydrogenated castor oil, PEG-200 hydrogenated glyceryl palmate.

Ethoxylated glycerol fatty acid esters are used in aqueous cleaning formulations for various purposes. Glycerol fatty acid esters with low degrees of ethoxylation (3-12 ethylene oxide units) usually serve as refatting agents for improving the feel on the skin after drying, glycerol fatty acid esters with a degree of ethoxylation of from about 30-50 serve as solubility promoters for nonpolar substances such as perfume oils. Highly ethoxylated glycerol fatty acid esters are used as thickeners. It is a common property of all of these substances that during application on the skin, upon dilution with water, they produce a particular feel on the skin.

Active Ingredients

It has been found that a very wide variety of active ingredients of varying solubility can be incorporated homogeneously into the cosmetic or dermatological compositions according to the invention. The substantivity of the active ingredients on skin and hair is greater from the described composition than from conventional surfactant-containing cleansing formulations.

According to the invention, the active ingredients (one or more compounds) can be chosen advantageously from the group consisting of acetylsalicylic acid, atropine, azulene, hydrocortisone and derivatives thereof, e.g. hydrocortisone-17 valerate, vitamins of the B and D series, in particular vitamin B₁, vitamin B₁₂, vitamin D, vitamin A and derivatives thereof, such as retinol palmitate, vitamin E or derivatives thereof, such as, for example, tocopherol acetate, vitamin C and derivatives thereof, such as, for example, ascorbyl glucoside, and also niacinamide, panthenol, bisabolol, polydocanol, unsaturated fatty acids, such as, for example, the essential fatty acids (usually referred to as vitamin F), in particular γ-linolenic acid, oleic acid, eicosapentaneoic acid, docosahexaenoic acid and derivatives thereof, chloramphenicol, caffeine, prostaglandins, thymol, camphor, squalene, extracts or other products of vegetable and animal origin, e.g. evening primrose oil, borage oil or blackcurrant seed oil, fish oils, cod liver oil, and also ceramides and ceramide-like compounds, incense extract, green tea extract, water lily extract, licorice extract, hamamelis, antidandruff active ingredients (e.g. selenium disulfide, zinc pyrithione, piroctone, olamine, climbazole, octopirox, polydocanol and combinations thereof), complexing active ingredients such as, for example, those comprising γ-oryzanol and calcium salts, such as calcium pantothenate, calcium chloride, calcium acetate.

It is also advantageous to choose the active ingredients from the group of refatting substances, for example purcellin oil, Eucerit® and Neocerit®.

The active ingredient or ingredients is/are particularly advantageously also chosen from the group of NO synthase inhibitors, particularly if the compositions according to the invention are to be used for the treatment and prophylaxis of the symptoms of intrinsic and/or extrinsic skin aging, and also for the treatment and prophylaxis of the harmful effects of ultraviolet radiation on the skin and the hair. A preferred NO synthase inhibitor is nitroarginine.

The active ingredient or the active ingredients are further advantageously chosen from the group comprising catechins and bile acid esters of catechins and aqueous or organic extracts from plants or parts of plants which have a content of catechins or bile acid esters of catechins, such as, for example, the leaves of the Theaceae family, in particular the Camellia sinensis (green tea) species. Of particular advantage are their typical ingredients (e.g. polyphenols and catechins, caffeine, vitamins, sugars, minerals, amino acids, lipids).

Catechins represent a group of compounds which are to be regarded as hydrogenated flavones or anthocyanidines and represent derivatives of “catechin” (catechol, 3,3′,4′,5,7-flavanepentaol, 2-(3,4-dihydroxyphenyl)chromane-3,5,7-triol). Epicatechin ((2R,3R)-3,3′,4′,5,7-flavanepentaol) is also an advantageous active ingredient for the purposes of the present invention.

Also advantageous are plant extracts with a content of catechins, in particular extracts of green tea, such as, for example, extracts from leaves of the plants of the Camellia spec. species, very particularly of the tea varieties Camellia sinenis, C. assamica, C. taliensis and C. inawadiensis and hybrids of these with, for example, Camellia japonica.

Preferred active ingredients are also polyphenols and catechins from the group (−)-catechin, (+)-catechin, (−)-catechin gallate, (−)-gallocatechin gallate, (+)-epicatechin, (−)-epicatechin, (−)-epicatechin gallate, (−)-epigallocatechin, (−)-epigallocatechin gallate.

Flavone and its derivatives (often also collectively called flavones) are also advantageous active ingredients for the purposes of the present invention. They are characterized by the following basic structure (substitution positions given):

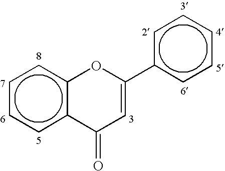

Some of the more important flavones which can also preferably be used in compositions according to the invention are listed in Table 2 below.

TABLE 2
Flavones
Table 2

| | OH substitution positions | | | | | | | |
|---|---|---|---|---|---|---|---|---|
| | 3 | 5 | 7 | 8 | 2′ | 3′ | 4′ | 5′ |
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Kaempferol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In nature, flavones generally occur in glycosylated form.

According to the invention, the flavonoids are preferably chosen chosen from the group of substances of the general formula

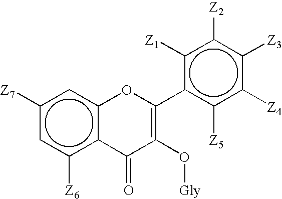

where Z₁ to Z₇, independently of one another, are chosen from the group consisting of H. OH, alkoxy and hydroxyalkoxy, where the alkoxy and/or hydroxyalkoxy groups may be branched or unbranched and have 1 to 18 carbon atoms, where Gly is chosen from the group of mono- and oligoglycoside radicals.

According to the invention, the flavonoids can, however, also be chosen advantageously from the group of substances of the general formula

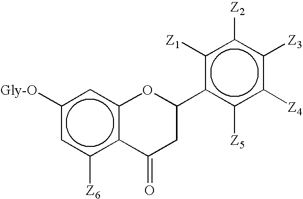

where Z₁ to Z₆, independently of one another, are chosen from the group consisting of H, OH, alkoxy and hydroxyalkoxy, where the alkoxy and hydroxyalkoxy groups may be branched and unbranched and have 1 to 18 carbon atoms, where Gly is chosen from the group of mono- and oligoglycoside radicals.

Preferably, such structures can be chosen from the group of substances of the general formula

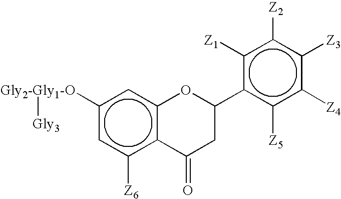

where Z₁ to Z₆, independently of one another, are as specified above and Gly₁, Gly₂ and Gly₃, independently of one another, are monoglycoside radicals or oligoglycoside radicals. Gly₂ and Gly₃ can also individually or together be saturations by hydrogen atoms.

Preferably, Gly₁, Gly₂ and Gly₃, independently of one another, are chosen from the group of hexosyl radicals, in particular rhamnosyl radicals and glucosyl radicals. However, other hexosyl radicals, for example allosyl, altrosyl, galactosyl, gulosyl, idosyl, mannosyl and talosyl are also to be used advantageously if appropriate.

It can also be advantageous according to the invention to use pentosyl radicals.

Advantageously, Z₁ to Z₅, independently of one another, are chosen from the group consisting of H, OH, methoxy, ethoxy and 2-hydroxyethoxy, and the flavone glycosides correspond to the general structural formula

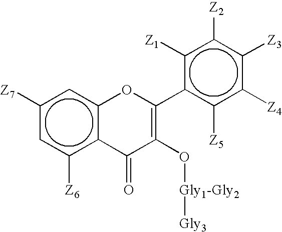

The flavone glycosides are particularly advantageously chosen from the group which is given by the following structure,

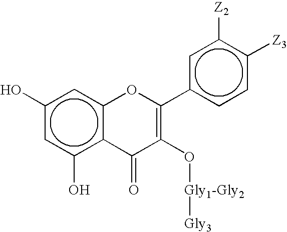

where Gly₁, Gly₂ and Gly₃, independently of one another, are monoglycoside radicals or oligoglycoside radicals. Gly₂ and Gly₃ can also individually or together be saturations by hydrogen atoms.

Preferably, Gly₁, Gly₂ and Gly₃, independently of one another, are chosen from the group of hexosyl radicals, in particular rhamnosyl radicals and glucosyl radicals. However, other hexosyl radicals, for example, allosyl, altrosyl, galactosyl, gulosyl, idosyl, mannosyl and talosyl, are also to be used advantageously if appropriate.

It may also be advantageous according to the invention to use pentosyl radicals.

It is particularly advantageous for the purposes of the present invention to choose the flavone glycoside or flavone glycosides from the group consisting of α-glucosylrutin, α-glucosylmyricetin, α-glucosylisoquercitrin, α-glucosylisoquercetin and α-glucosylquercitrin.

Further advantageous active ingredients are sericoside, pyridoxol, vitamin K, biotin and aroma substances.

Furthermore, the active ingredients (one or more compounds) can also very advantageously be chosen from the group of hydrophilic active ingredients, in particular from the following group:

α-hydroxy acids, such as lactic acid or salicylic acid and salts thereof, such as, for example, Na lactate, Ca lactate, TEA lactate, urea, allantoin, serine, sorbitol, glycerol, milk proteins, panthenol, chitosan.

The list of specified active ingredients and active ingredient combinations which can be used in the compositions according to the invention is not of course intended to be limiting. The active ingredients can be used individually or in any combinations with one another.

The amount of such active ingredients (one or more compounds) in the compositions according to the invention is preferably 0.001 to 30% by weight, particularly preferably 0.05 to 20% by weight, in particular 1 to 10% by weight, based on the total weight of the composition.

The specified and further active ingredients which can be used in the compositions according to the invention are given in DE 103 18 526 A1 on pages 12 to 17, which is hereby incorporated in its entirety at this point by reference.

Pearlescent Waxes

Suitable pearlescent waxes are, for example: alkylene glycol esters, specifically ethylene glycol distearate; fatty acid alkanolamides, specifically coconut fatty acid diethanolamide; partial glycerides, specifically stearic acid monoglyceride; esters of polybasic, optionally hydroxy-substituted carboxylic acids with fatty alcohols having 6 to 22 carbon atoms, specifically long-chain esters of tartaric acid; fatty substances, such as, for example, fatty alcohols, fatty ketones, fatty aldehydes, fatty ethers and fatty carbonates which have a total of at least 24 carbon atoms, specifically laurone and distearyl ether; fatty acids, such as stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 10 hydroxyl groups, and mixtures thereof.

The compositions according to the invention can also comprise glitter substances and/or other effect substances (e.g. color streaks).

Emulsifiers

The cosmetic compositions according to the invention are in the form of emulsions in a preferred embodiment of the invention. Such emulsions are prepared by known methods. Besides the W/W emulsion polymers, the emulsions can also comprise fatty alcohols, fatty acid esters and, in particular, fatty acid triglycerides, fatty acids, lanolin and derivatives thereof, natural or synthetic oils or waxes and emulsifiers in the presence of water. The choice of additives specific to the type of emulsion and the preparation of suitable emulsions is described, for example, in Schrader, Grundlagen und Rezepturen der Kosmetika [Fundamentals and formulations of cosmetics], Hüthig Buch Verlag, Heidelberg, 2nd edition, 1989, third part, which is hereby expressly incorporated by reference.

A suitable emulsion, e.g. for a skin cream, generally comprises an aqueous phase which is emulsified in an oil or fatty phase by means of a suitable emulsifier system.

The fraction of emulsifier system in this type of emulsion is preferably about 4 to 35% by weight, based on the total weight of the emulsion. The fraction of the fatty phase is preferably about 20 to 60% by weight. Preferably, the fraction of the aqueous phase is about 20 and 70%, in each case based on the total weight of the emulsion.

Suitable emulsifiers are, for example, nonionogenic surfactants from at least one of the following groups:
(1) addition products of from 2 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group;
(2) C12/18 fatty acid mono- and diesters of addition products of from 1 to 30 mol of ethylene oxide onto glycerol;
(3) glycerol mono- and diesters and sorbitan mono- and diesters of saturated and unsaturated fatty acids having 6 to 22 carbon atoms and ethylene oxide addition products thereof;
(4) alkyl mono- and oligoglycosides having 8 to 22 carbon atoms in the alkyl radical and ethoxylated analogs thereof;
(5) addition products of from 15 to 60 mol of ethylene oxide onto oils, for example onto castor oil and/or hydrogenated castor oil;
(6) polyol, and in particular polyglycerol, esters, such as, for example, polyglycerol polyricinoleate, polyglycerol poly-12-hydroxystearate or polyglycerol dimerate. Mixtures of compounds from two or more of these classes of substances are likewise suitable;
(7) addition products of from 2 to 15 mol of ethylene oxide onto castor oil and/or hydrogenated castor oil;
(8) partial esters based on linear, branched, unsaturated or saturated C₆/₂₂-fatty acids, ricinoleic acid, and 12-hydroxystearic acid and glycerol, polyglycerol, pentaerythritol, dipentaerythritol, sugar alcohols (e.g. sorbitol), alkyl glucosides (e.g. methyl glucoside, butyl glucoside, lauryl glucoside), and polyglucosides (e.g. cellulose);
(9) mono-, di- and trialkyl phosphates, and mono-, di- and/or tri-PEG alkyl phosphates and salts thereof;
(10) wool wax alcohols;
(11) polysiloxane-polyalkyl-polyether copolymers and corresponding derivatives;
(12) mixed esters of pentaerythritol, fatty acids, citric acid and fatty alcohols according to DE-C 1165574 and/or mixed esters of fatty acids having 6 to 22 carbon atoms, methylglycose and polyols, preferably glycerol or polyglycerol and
(13) polyalkylene glycols.

The addition products of ethylene oxide and/or of propylene oxide onto fatty alcohols, fatty acids, alkylphenols, glycerol mono- and diesters, and sorbitan mono- and diesters of fatty acids or onto castor oil are known, commercially available products. These are homolog mixtures whose average degree of alkoxylation corresponds to the ratio of the quantitative amounts of ethylene oxide and/or propylene oxide and substrate with which the addition reaction is carried out. C₁₂ to C₁₈-fatty acid mono- and diesters of addition products of ethylene oxide onto glycerol are known from DE-C 2024051 as refatting agents for cosmetic compositions. C₈ to C₁₈-alkyl mono- and oligoglycosides, their preparation and their use are known from the prior art. Their preparation takes place in particular by reacting glucose or oligosaccharides with primary alcohols having 8 to 18 carbon atoms. As regards the glycoside ester, both monoglycosides in which a cyclic sugar radical is bonded glycosidically to the fatty alcohol, and also oligomeric glycosides with a degree of oligomerization up to preferably about 8 are suitable. The degree of oligomerization here is a statistical average value which is based on a homolog distribution customary for such technical products.

In addition, zwitterionic surfactants can be used as emulsifiers. Zwitterionic surfactants is the term used to describe those surface-active compounds which carry at least one quaternary ammonium group and at least one carboxylate and/or one sulfonate group in the molecule. Particularly suitable zwitterionic surfactants are the so-called betaines, such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxylmethyl-3-hydroxyethylimidazolines having in each case 8 to 18 carbon atoms in the alkyl or acyl group, and cocoacylaminoethyl hydroxyethylcarboxymethyl glycinate.

Of particular preference is the fatty acid amide derivative known under the CTFA name Cocamidopropyl Betaine. Likewise suitable emulsifiers are ampholytic surfactants. Ampholytic surfactants are understood as meaning those surface-active compounds which, apart from a C₈ to C₁₈-alkyl or -acyl group in the molecule, comprise at least one free amino group and at least one —COOH and/or —SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group. Particularly preferred ampholytic surfactants are N-cocoalkylaminopropionate, cocoacylaminoethylaminopropionate and C₁₂ to C₁₈-acylsarcosine.

Besides the ampholytic emulsifiers, quaternary emulsifiers are also suitable, particular preference being given to those of the ester quat type, preferably methyl-quaternized difatty acid triethanolamine ester salts.

Perfume Oils

If appropriate, the cosmetic compositions according to the invention can comprise perfume oils. Perfume oils which may be mentioned are, for example, mixtures of natural and synthetic fragrances. Natural fragrances are extracts from flowers (lily, lavender, rose, jasmine, neroli, ylang-ylang), stems and leaves (geranium, patchouli, petitgrain), fruits (anise, coriander, cumin, juniper), fruit peels (bergamot, lemon, orange), roots (mace, angelica, celery, cardamom, costus, iris, calmus), woods (pine wood, sandalwood, guaiac wood, cedar wood, rose wood), herbs and grasses (tarragon, lemon grass, sage, thyme), needles and branches (spruce, fir, pine, dwarf-pine), resins and balsams (galbanum, elemi, benzoin, myrrh, olibanum, opoponax). Also suitable are animal raw materials, such as, for example, civet and castoreum. Typical synthetic fragrance compounds are products of the ester, ether, aldehyde, ketone, alcohol and hydrocarbon type. Fragrance compounds of the ester type are, for example, benzyl acetate, phenoxyethyl isobutyrate, 4-tert-butyl cyclohexylacetate, linalyl acetate, dimethylbenzylcarbinyl acetate, phenylethyl acetate, linalyl benzoate, benzyl formate, ethylmethyl phenylglycinate, allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamenaldehyde, hydroxycitronellal, lilial and bourgeonal, the ketones include, for example, the ionones, cc-isomethylionene and methyl cedryl ketone, the alcohols include anethol, citronellol, eugenol, isoeugenol, geraniol, linalool, phenylethyl alcohol and terioneol, the hydrocarbons include primarily the terpenes and balsams. However, preference is given to using mixtures of different fragrances which together produce a pleasing scent note. Essential oils of lower volatility, which are mostly used as aroma components, are also suitable as perfume oils, e.g. sage oil, camomile oil, oil of cloves, melissa oil, mint oil, cinnamon leaf oil, linden blossom oil, juniperberry oil, vetiver oil, olibanum oil, galbanum oil, labolanum oil and lavandin oil. Preference is given to using bergamot oil, dihydromyrcenol, lilial, lyral, citronellol, phenylethyl alcohol, α-hexylcinnamaldehyde, geraniol, benzyl acetone, cyclamenaldehyde, linalool, Boisambrene®Forte, ambroxan, indole, hedione, sandelice, citrus oil, mandarin oil, orange oil, allyl amyl glycolate, cyclovertal, lavandin oil, clary sage oil, β-damascone, geranium oil bourbon, cyclohexyl salicylate, Vertofix®Coeur, Iso-E-Super®, Fixolide®NP, evernyl, iraldein gamma, phenylacetic acid, geranyl acetate, benzyl acetate, rose oxide, romillat, irotyl and floramat alone or in mixtures.

Pigments

If appropriate, the cosmetic compositions according to the invention further comprise pigments.

The pigments are present in the product mass in undissolved form and may be present in an amount of from 0.01 to 25% by weight, particularly preferably from 5 to 15% by weight. The preferred particle size is 1 to 200 μm, in particular 3 to 150 μm, particularly preferably 10 to 100 μm. The pigments are colorants which are virtually insoluble in the application medium and may be inorganic or organic. Inorganic-organic mixed pigments are also possible. Preference is given to inorganic pigments. The advantage of the inorganic pigments is their excellent fastness to light, weather and temperature. The inorganic pigments can be of natural origin, for example prepared from chalk, ocher, umber, green earth, burnt sienna or graphite. The pigments may be white pigments, such as, for example, titanium dioxide or zinc oxide, black pigments, such as, for example, iron oxide black, colored pigments, such as, for example, ultramarine or iron oxide red, luster pigments, metal effect pigments, pearlescent pigments, and fluorescent or phosphorescent pigments, where preferably at least one pigment is a colored, non-white pigment.

Metal oxides, hydroxides and oxide hydrates, mixed phase pigments, sulfur-containing silicates, metal sulfides, complex metal cyanides, metal sulfates, chromates and molybdates, and the metals themselves (bronze pigments) are suitable. Of particular suitability are titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (C177289), iron blue (ferric ferrocyanide, CI 77510), carmine (cochineal).

Particular preference is given to pearlescent and color pigments based on mica which are coated with a metal oxide or a metal oxychloride such as titanium dioxide or bismuth oxychloride, and, if appropriate, further color-imparting substances such as iron oxides, iron blue, ultramarine, carmine etc. and where the color can be determined by varying the layer thickness. Such pigments are sold, for example, under the trade names Rona®, Colorona®, Dichrona® and Timiron® (Merck).

Organic pigments are, for example, the natural pigments sepia, gamboge, bone charcoal, cassel brown, indigo, chlorophyll and other plant pigments. Synthetic organic pigments are, for example, azo pigments, anthraquinoids, indigoids, dioxazine, quinacridone, phthalocyanine, isoindolinone, perylene and perinone, metal complex, alkali blue and diketopyrrolopyrrole pigments.

In one embodiment, the composition according to the invention comprises 0.01 to 10% by weight, particularly preferably from 0.05 to 5% by weight, of at least one particulate substance. Suitable substances are, for example, substances which are solid at room temperature (25° C.) and are in the form of particles. For example, silica, silicates, aluminates, clay earths, mica, salts, in particular inorganic metal salts, metal oxides, e.g. titanium dioxide, minerals and polymer particles are suitable.

The particles are present in the composition undissolved, preferably in stably dispersed form and, following application to the application surface and evaporation of the solvent, can settle out in solid form.

Preferred particulate substances are silica (silica gel, silicon dioxide) and metal salts, in particular inorganic metal salts, particular preference being given to silica. Metal salts are, for example, alkali metal or alkaline earth metal halides, such as sodium chloride or potassium chloride; alkali metal or alkaline earth metal sulfates, such as sodium sulfate or magnesium sulfate.

Polymers

The cosmetic compositions according to the invention can also comprise additional polymers.

Suitable polymers are, for example, cationic polymers with the INCI name Polyquaternium, e.g. copolymers of vinylpyrrolidone/N-vinylimidazolium salts (Luviquate® FC, Luviquat® HM, Luviquate® MS, Luviquate® Care, Luviquate® UltraCare), copolymers of N-vinylpyrrolidone/dimethylaminoethyl methacrylate quaternized with diethyl sulfate (Luviquate® PQ 11), copolymers of N-vinylcaprolactam/N-vinylpyrrolidone/N-vinylimidazolium salts (Luviquate® Hold); cationic cellulose derivatives (polyquaternium-4 and -10), acrylamido copolymers (polyquaternium-7) and chitosan. Suitable cationic (quaternized) polymers are also Merquate® (polymer based on dimethyldiallylammonium chloride), Gafquate® (quaternary polymers which are formed by reacting polyvinylpyrrolidone with quaternary ammonium compounds), polymer JR (hydroxyethylcellulose with cationic groups) and cationic polymers based on plants, e.g. guar polymers, such as the Jaguar® grades from Rhodia.

Further suitable polymers are also neutral polymers, such as polyvinylpyrrolidone, copolymers of N-vinylpyrrolidone and vinyl acetate and/or vinyl propionate and/or stearyl (meth)acrylate, polysiloxanes, polyvinylcaprolactam and other copolymers with N-vinylpyrrolidone, polyethyleneimines and salts thereof, polyvinylamines and salts thereof, cellulose derivatives, polyaspartic acid salts and derivatives. These include, for example, Luviflex® Swing (partially hydrolyzed copolymer of polyvinyl acetate and polyethylene glycol, BASF) or Kollicoat® IR.

Suitable polymers are also the (meth)acrylamide copolymers described in WO 03/092640, in particular those described as Examples 1 to 50 (Table 1, page 40 ff.) and Examples 51 to 65 (Table 2, page 43), which is hereby incorporated in its entirety at this point by reference.

Suitable polymers are also nonionic, water-soluble or water-dispersible polymers or oligomers, such as polyvinylcaprolactam, e.g. Luviskol® Plus (BASF), or polyvinylpyrrolidone and copolymers thereof, in particular with vinyl esters, such as vinyl acetate, e.g. Luviskol® VA 37 (BASF); polyamides, e.g. based on itaconic acid and aliphatic diamines, as are described, for example, in DE-A-43 33 238.

Suitable polymers are also amphoteric or zwitterionic polymers, such as the octylacrylamide/methyl methacrylate/tert-butylaminoethyl methacrylate/2-hydroxypropyl methacrylate copolymers obtainable under the names Amphomer® (National Starch), and zwitterionic polymers as are disclosed, for example, in the German patent applications DE 39 29 973, DE 21 50 557, DE 28 17 369 and DE 37 08 451. Acrylamidopropyltrimethylammonium chloride/acrylic acid or methacrylic acid copolymers and alkali metal and ammonium salts thereof are preferred zwitterionic polymers. Further suitable zwitterionic polymers are methacroylethylbetaine/methacrylate copolymers, which are commercially available under the name Amersette® (AMERCHOL), and copolymers of hydroxyethyl methacrylate, methyl methacrylate, N,N-dimethylaminoethyl methacrylate and acrylic acid (Jordapon®).

Suitable polymers are also nonionic, siloxane-containing, water-soluble or-dispersible polymers, e.g. polyether siloxanes, such as Tegopren® (Goldschmidt) or Belsil® (Wacker).

Furthermore, biopolymers are also suitable, i.e. polymers which are obtained from naturally renewable raw materials and are constructed from natural monomer building blocks, e.g. cellulose derivatives, chitin, chitosan, DNA, hyaluronic acid and RNA derivatives.

Further compositions according to the invention comprise at least one further water-soluble polymer, in particular chitosans (poly(D-glucosamines)) of varying molecular weight and/or chitosan derivatives.

Anionic Polymers

Further polymers suitable for the compositions according to the invention are copolymers containing carboxylic acid groups. These are polyelectrolytes with a relatively large number of anionically dissociable groups in the main chain and/or one side chain. They are capable of forming polyelectrolyte complexes (symplexes) with the copolymers A).

In a preferred embodiment, the polyelectrolyte complexes used in compositions according to the invention have an excess of anionogenic/anionic groups.

Besides at least one of the abovementioned copolymers A), the polyelectrolyte complexes also comprise at least one polymer containing acid groups.

The polyelectrolyte complexes preferably comprise copolymer(s) A) and polymers containing acid groups in a quantitative ratio by weight of from about 50:1 to 1:20, particularly preferably from 20:1 to 1:5.

Suitable polymers containing carboxylic acid groups are obtainable, for example, by free-radical polymerization of α,β-ethylenically unsaturated monomers. Here, monomers m1) are used which comprise at least one free-radically polymerizable, α,β-ethylenically unsaturated double bond and at least one anionogenic and/or anionic group per molecule.

Suitable polymers containing carboxylic acid groups are also polyurethanes containing carboxylic acid groups. Preferably, the monomers are chosen from monoethylenically unsaturated carboxylic acids, sulfonic acids, phosphonic acids and mixtures thereof.

The monomers m1) include monoethylenically unsaturated mono- and dicarboxylic acids having 3 to 25, preferably 3 to 6, carbon atoms, which can also be used in the form of their salts or anhydrides. Examples thereof are acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid. The monomers also include the monoesters of monoethylenically unsaturated dicarboxylic acids having 4 to 10, preferably 4 to 6, carbon atoms, e.g. of maleic acid, such as monomethyl maleate. The monomers also include monoethylenically unsaturated sulfonic acids and phosphonic acids, for example vinylsulfonic acid, allylsulfonic acid, sulfoethyl acrylate, sulfoethyl methacrylate, sulfopropyl acrylate, su Ifopropyl methacrylate, 2-hydroxy-3-acryloxypropylsulfonic acid, 2-hydroxy-3-methacryloxypropylsulfonic acid, styrenesulfonic acid, 2-acrylamido-2-methylpropanesulfonic acid, vinylphosphonic acid and allylphosphonic acid. The monomers also include the salts of the abovementioned acids, in particular the sodium, potassium and ammonium salts, and the salts with the abovementioned amines. The monomers can be used as they are or as mixtures with one another. The weight fractions given all refer to the acid form.

Preferably, the monomer m1) is chosen from acrylic acid, methacrylic acid, ethacrylic acid, α-chloroacrylic acid, crotonic acid, maleic acid, maleic anhydride, fumaric acid, itaconic acid, citraconic acid, mesaconic acid, glutaconic acid, aconitic acid and mixtures thereof, particularly preferably acrylic acid, methacrylic acid and mixtures thereof.

The abovementioned monomers m1) can in each case be used individually or in the form of any mixtures.

Of suitability in principle as comonomers for the preparation of the polymers containing carboxylic acid groups are the compounds a) to d) given above as components of copolymer A) with the proviso that the molar fraction of anionogenic and anionic groups which comprise the copolymerized polymer containing carboxylic acid groups is larger than the molar fraction of cationogenic and cationic groups.

In a preferred embodiment, the polymers containing carboxylic acid groups comprise at least one monomer in copolymerized form which is chosen from the abovementioned crosslinkers.

Furthermore, the polymers containing carboxylic acid groups preferably comprise at least one monomer m2) in copolymerized form which is chosen from compounds of the general formula (VI)

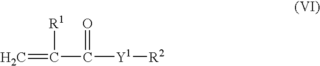

in which
R¹ is hydrogen or C₁-C₈-alkyl,
Y¹ is O NH or NR³, and
R² and R³ independently of one another are C₁-C₃₀-alkyl or C₅-C₈-cycloalkyl, where the alkyl groups may be interrupted by up to four nonadjacent heteroatoms or heteroatom-containing groups which are chosen from O, S and NH.

Preferably, R¹ in the formula VI is hydrogen or C₁-C₄-alkyl, in particular hydrogen, methyl or ethyl. Preferably, R² in the formula VI is C₁-C₈-alkyl, preferably methyl, ethyl, n-butyl, isobutyl, tert-butyl or a group of the formula —CH₂—CH₂—NH—C(CH₃)₃. If R³ is alkyl, then it is preferably C₁-C₄-alkyl, such as methyl, ethyl, n-propyl, n-butyl, isobutyl or tert-butyl.

Suitable monomers m2) are methyl (meth)acrylate, methyl ethacrylate, ethyl (meth)acrylate, ethyl ethacrylate, tert-butyl (meth)acrylate, tert-butyl ethacrylate, n-octyl (meth)acrylate, 1,1,3,3-tetramethylbutyl (meth)acrylate, ethylhexyl (meth)acrylate, n-nonyl (meth)acrylate, n-decyl (meth)acrylate, n-undecyl (meth)acrylate, tridecyl (meth)acrylate, myristyl (meth)acrylate, pentadecyl (meth)acrylate, palmityl (meth)acrylate, heptadecyl (meth)acrylate, nonadecyl (meth)acrylate, arachinyl (meth)acrylate, behenyl (meth)acrylate, lignocerenyl (meth)acrylate, cerotinyl (meth)acrylate, melissinyl (meth)acrylate, palmitoleinyl (meth)acrylate, oleyl (meth)acrylate, linolyl (meth)acrylate, linolenyl (meth)acrylate, stearyl (meth)acrylate, lauryl (meth)acrylate and mixtures thereof.

Suitable monomers m2) are also acrylamide, methacrylamide, N-methyl(meth)-acrylamide, N-ethyl(meth)acrylamide, N-propyl(meth)acrylamide, N-(n-butyl)(meth)-acrylamide, N-(tert-butyl)(meth)acrylamide, N,N-dimethyl(meth)acrylamide, N,N-diethyl(meth)acrylamide, piperidinyl(meth)acrylamide and morpholinyl-(meth)acrylamide, N-(n-octyl)(meth)acrylamide, N-(1,1,3,3-tetramethylbutyl)-(meth)acrylamide, N-ethylhexyl(meth)acrylamide, N-(n-nonyl)(meth)acrylamide, N-(n-decyl)(meth)acrylamide, N-(n-undecyl)(meth)acrylamide, N-tridecyl-(meth)acrylamide, N-myristyl(meth)acrylamide, N-pentadecyl(meth)acrylamide, N-palmityl(meth)acrylamide, N-heptadecyl(meth)acrylamide, N-nonadecyl(meth)-acrylamide, N-arachinyl(meth)acrylamide, N-behenyl(meth)acrylamide, N-lignocerenyl(meth)acrylamide, N-cerotinyl(meth)acrylamide, N-melissinyl(meth)-acrylamide, N-palmitoleinyl(meth)acrylamide, N-oleyl(meth)acrylamide, N-linolyl-(meth)acrylamide, N-linolenyl(meth)acrylamide, N-stearyl(meth)acrylamide and N-lauryl(meth)acrylamide.

Furthermore, the polymers containing carboxylic acid groups preferably comprise at least one monomer m3) in copolymerized form which is chosen from compounds of the general formula VII

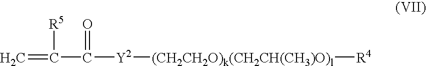

in which

the order of the alkylene oxide units is arbitrary,
k and I independently of one another are an integer from 0 to 1000, where the sum of k and I is at least 5,
R⁴ is hydrogen, C₁-C₃₀-alkyl or C₅-C₈-cycloalkyl,
R⁵ is hydrogen or C₁-C₈-alkyl,
Y² is O or NR⁶, where R⁶ is hydrogen, C₁-C₃₀-alkyl or C₅-C₈-cycloalkyl.

Preferably, in the formula VII, k is an integer from 1 to 500, in particular 3 to 250. Preferably, I is an integer from 0 to 100. Preferably, R⁵ is hydrogen, methyl, ethyl, n-propyl, isopropyl, n-butyl, sec-butyl, tert-butyl, n-pentyl or n-hexyl, in particular hydrogen, methyl or ethyl. Preferably R⁴ in the formula VII is hydrogen, methyl, ethyl, n-propyl, isopropyl, n-butyl, sec-butyl, n-pentyl, n-hexyl, octyl, 2-ethylhexyl, decyl, lauryl, palmityl or stearyl. Preferably, Y² in the formula VII is O or NH.

Suitable polyether acrylates VII) are, for example, the polycondensation products of the abovementioned α,β-ethylenically unsaturated mono- and/or dicarboxylic acids and the acid chlorides, amides and anhydrides thereof with polyetherols. Suitable polyetherols can be readily prepared by reacting ethylene oxide, 1,2-propylene oxide and/or epichlorohydrin with a starter molecule, such as water or a short-chain alcohol R⁴—OH. The alkylene oxides can be used individually, alternately one after the other or as a mixture. The polyether acrylates VII) can be used on their own or in mixtures for preparing the polymers used according to the invention. Suitable polyether acrylates II) are also urethane (meth)acrylates with alkylene oxide groups. Such compounds are described in DE 198 38 851 (component e2)), which is hereby incorporated in its entirety by reference.

Anionic polymers preferred as polymers containing carboxylic acid groups are, for example, homopolymers and copolymers of acrylic acid and methacrylic acid and salts thereof. These also include crosslinked polymers of acrylic acid, as are obtainable under the INCI name Carbomer. Such crosslinked homopolymers of acrylic acid are available commercially, for example, under the name Carbopol® from Noveon. Preference is also given to hydrophobically modified crosslinked polyacrylate polymers such as Carbopol® Ultrez 21 from Noveon.

Further examples of suitable anionic polymers are copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of (meth)acrylic acid and polyether acrylates, where the polyether chain is terminated with a C₈-C₃₀-alkyl radical. These include, for example, acrylate/beheneth-25 methacrylate copolymers, which are available under the name Aculyn® from Rohm und Haas. Particularly suitable polymers are also copolymers of t-butyl acrylate, ethyl acrylate, methacrylic acid (e.g. Luvimer® 100P, Luvimer® Pro55), copolymers of ethyl acrylate and methacrylic acid (e.g. Luvimer® MAE), copolymers of N-tert-butylacrylamide, ethyl acrylate, acrylic acid (Ultrahold® 8, Ultrahold® Strong), copolymers of vinyl acetate, crotonic acid and, if appropriate, further vinyl esters (e.g. Luviset® grades), maleic anhydride copolymers, if appropriate reacted with alcohol, anionic polysiloxanes, e.g. carboxyfunctional ones, t-butyl acrylate, methacrylic acid (e.g. Luviskol® VBM), copolymers of acrylic acid and methacrylic acid with hydrophobic monomers, such as, for example, C₄-C₃₀-alkyl esters of meth(acrylic acid), C₄-C₃₀-alkyl vinyl esters, C₄-C₃₀-alkyl vinyl ethers and hyaluronic acid. Examples of anionic polymers are also vinyl acetate/crotonic acid copolymers, as are sold, for example, under the names Resyn® (National Starch) and Gafset® (GAF), and vinylpyrrolidone/vinyl acrylate copolymers obtainable, for example, under the trade name Luviflex® (BASF). Further suitable polymers are the vinylpyrrolidone/acrylate terpolymer obtainable under the name Luviflex® VBM-35 (BASF) and polyamides containing sodium sulfonate or polyesters containing sodium sulfonate.

In addition, the group of suitable anionic polymers comprises, by way of example, Balance® CR (National Starch; acrylate copolymer), Balance® 0/55 (National Starch; acrylate copolymer), Balance® 47 (National Starch; octylacrylamide/acrylate/butylaminoethyl methacrylate copolymer), Aquaflex® FX 64 (ISP; isobutylene/ethylmaleimide/hydroxyethylmaleimide copolymer), Aquaflex® SF-40 (ISP/National Starch; VP/vinylcaprolactam/DMAPA acrylate copolymer), Allianz® LT-120 (ISP/Rohm & Haas; acrylate/C1-2 succinate/hydroxyacrylate copolymer), Aquarez® HS (Eastman; polyester-1), Diaformer® Z-400 (Clariant; methacryloylethylbetaine/methacrylate copolymer), Diaformer® Z-711 (Clariant; methacryloylethyl N-oxide/methacrylate copolymer), Diaformer® Z-712 (Clariant; methacryloylethyl N-oxide/methacrylate copolymer), Omnirez® 2000 (ISP; monoethyl ester of poly(methyl vinyl ether/maleic acid in ethanol), Amphomer® HC (National Starch; acrylate/octylacrylamide copolymer), Amphomer® 28-4910 (National Starch; octylacrylamide/acrylate/butylaminoethyl methacrylate copolymer), Advantage® HC 37 (ISP; terpolymer of vinylcaprolactam/vinylpyrrolidone/dimethylaminoethyl methacrylate), Advantage® LC55 and LC80 or LC A and LC E, Advantage® Plus (ISP; VA/butyl maleate/isobornyl acrylate copolymer), Aculyne® 258 (Rohm & Haas; acrylate/hydroxy ester acrylate copolymer), Luviset® P.U.R. (BASF, polyurethane-1), Luviflex® Silk (BASF), Eastman® AQ 48 (Eastman), Styleze® CC-10 (ISP; VP/DMAPA acrylates copolymer), Styleze® 2000 (ISP; VP/acrylates/lauryl methacrylate copolymer), DynamX® (National Starch; polyurethane-14 AMP-acrylates copolymer), Resyn XP® (National Starch; acrylates/octylacrylamide copolymer), Fixomer® A-30 (Ondeo Nalco; polymethacrylic acid (and) acrylamidomethylpropanesulfonic acid), Fixate® G-100 (Noveon; AMP-acrylates/allyl methacrylate copolymer).

Suitable polymers containing carboxylic acid groups are also the terpolymers of vinylpyrrolidone, C₁-C₁₀-alkyl, cycloalkyl and aryl (meth)acrylates and acrylic acid described in U.S. Pat. No. 3,405,084. Suitable polymers containing carboxylic acid groups are also the terpolymers of vinylpyrrolidone, tert-butyl (meth)acrylate and (meth)acrylic acid described in EP-A-0 257 444 and EP-A-0 480 280. Suitable polymers containing carboxylic acid groups are also the copolymers described in DE-A-42 23 066 which comprise, in copolymerized form, at least one (meth)acrylic ester, (meth)acrylic acid and N-vinylpyrrolidone and/or N-vinylcaprolactam. The disclosure of these documents is hereby incorporated in its entirety by reference.

The abovementioned polymers containing carboxylic acid groups are prepared by known processes, for example solution, precipitation, suspension or emulsion polymerization, as described above for the copolymers A).

Suitable polymers containing carboxylic acid groups are also polyurethanes containing carboxylic acid groups.

EP-A-636361 discloses suitable block copolymers with polysiloxane blocks and polyurethane/polyurea blocks which have carboxylic acid and/or sulfonic acid groups. Suitable silicone-containing polyurethanes are also described in WO 97/25021 and EP-A-751 162.

Suitable polyurethanes are also described in DE-A-42 25 045, which is hereby incorporated in its entirety by reference.

The acid groups of the polymers containing carboxylic acid groups can be partially or completely neutralized. At least some of the acid groups are then present in deprotonated form, the counterions preferably being chosen from alkali metal ions, such as Na⁺, K⁺, ammonium ions and organic derivatives thereof etc.

Propellants (Propellant Gases)

If the compositions according to the invention are to be provided as aerosol spray, propellants are necessary. Suitable propellants (propellant gases) are the customary propellants, such as n-propane, isopropane, n-butane, isobutane, 2,2-dimethylbutane, n-pentane, isopentane, dimethyl ether, difluoroethane, fluorotrichloromethane, dichlorodifluoromethane or dichlorotetrafluoroethane, HFC 152 A or mixtures thereof. Hydrocarbons, in particular propane, n-butane, n-pentane and mixtures thereof, and also dimethyl ether and difluoroethane are primarily used. If appropriate, one or more of the specified chlorinated hydrocarbons are co-used in propellant mixtures, but only in small amounts, for example up to 20% by weight, based on the propellant mixture.

The hair cosmetic preparations according to the invention are also suitable for pump spray preparations without the addition of propellants and also for aerosol sprays with customary compressed gases such as nitrogen, compressed air or carbon dioxide as propellant.

Surfactants

The compositions according to the invention can also comprise surfactants. Surfactants which may be used are anionic, cationic, nonionic and/or amphoteric surfactants.

Advantageous anionic surfactants for the purposes of the present invention are acylamino acids and salts thereof, such as
acyl glutamates, in particular sodium acylglutamate
sarcosinates, for example myristoyl sarcosine, TEA lauroyl sarcosinate, sodium lauroyl sarcosinate and sodium cocoyl sarcosinate,
   sulfonic acids and salts thereof, such as
acyl isethionates, for example sodium or ammonium cocoyl isethionate
   sulfosuccinates, for example dioctyl sodium sulfosuccinate, disodium laureth sulfosuccinate, disodium lauryl sulfosuccinate and disodium undecylenamido MEA sulfosuccinate, disodium PEG-5 lauryl citrate sulfosuccinate and derivatives,
      and sulfuric esters, such as
alkyl ether sulfate, for example sodium, ammonium, magnesium, MIPA, TIPA laureth sulfate, sodium myreth sulfate and sodium C₁₂₋₁₃ pareth sulfate,
alkyl sulfates, for example sodium, ammonium and TEA lauryl sulfate.

Further advantageous anionic surfactants are
taurates, for example sodium lauroyl taurate and sodium methyl cocoyl taurate,
ether carboxylic acids, for example sodium laureth-13 carboxylate and sodium PEG-6 cocamide carboxylate, sodium PEG-7 olive oil carboxylate
phosphoric esters and salts, such as, for example, DEA oleth-10 phosphate and dilaureth-4 phosphate,
alkylsulfonates, for example sodium cocomonoglyceride sulfate, sodium C₁₂₋₁₄ olefinsulfonate, sodium lauryl sulfoacetate and magnesium PEG-3 cocamide sulfate,
acyl glutamates, such as Di-TEA palmitoyl aspartate and sodium caprylic/capric glutamate,
acyl peptides, for example palmitoyl hydrolyzed milk protein, sodium cocoyl hydrolyzed soybean protein and sodium/potassium cocoyl hydrolyzed collagen
   and carboxylic acids and derivatives, such as,

for example lauric acid, aluminum stearate, magnesium alkanolate and zinc undecylenate, ester carboxylic acids, for example calcium stearoyl lactylate, laureth-6 citrate and sodium PEG-4 lauramide carboxylate
alkylarylsulfonates.

Advantageous cationic surfactants for the purposes of the present invention are quaternary surfactants. Quaternary surfactants comprise at least one N atom which is covalently bonded to 4 alkyl or aryl groups. Alkylbetaine, alkylamidopropylbetaine and alkylamidopropylhydroxysultaine, for example, are advantageous.

Further advantageous cationic surfactants for the purposes of the present invention are also
alkylamines,
alkylimidazoles and
ethoxylated amines
   and in particular salts thereof.

Advantageous amphoteric surfactants for the purposes of the present invention are acyl-/dialkylethylenediamines, for example sodium acyl amphoacetate, disodium acyl amphodipropionate, disodium alkyl amphodiacetate, sodium acyl amphohydroxypropylsulfonate, disodium acyl amphodiacetate, sodium acyl amphopropionate, and N-coconut fatty acid amidoethyl-N-hydroxyethyl glycinate sodium salts.

Further advantageous amphoteric surfactants are N-alkylamino acids, for example aminopropylalkylglutamide, alkylaminopropionic acid, sodium alkyl imidodipropionate and lauroamphocarboxyglycinate.

Advantageous active nonionic surfactants for the purposes of the present invention are
alkanolamides, such as cocamides MEA/DEA/MIPA,
esters which are formed by esterification of carboxylic acids with ethylene oxide, glycerol, sorbitan or other alcohols,
ethers, for example ethoxylated alcohols, ethoxylated lanolin, ethoxylated polysiloxanes, propoxylated POE ethers, alkyl polyglycosides, such as laurylglucoside, decyl glycoside and cocoglycoside, glycosides with an HLB value of at least 20 (e.g. Belsil®SPG 128V (Wacker)).

Further advantageous nonionic surfactants are alcohols and amine oxides, such as cocoamidopropylamine oxide.

Among the alkyl ether sulfates, sodium alkyl ether sulfates based on di- or triethoxylated lauryl and myristyl alcohol in particular are preferred. They are significantly superior to the alkyl sulfates with regard to insensitivity toward water hardness, ability to be thickened, solubility at low temperatures and, in particular, skin and mucosa compatibility. Lauryl ether sulfate has better foaming properties than myristyl ether sulfate, but is inferior to this in terms of mildness.

Alkyl ether carboxylates with an average and particularly with a relatively high actually belong to the mildest surfactants, but exhibit poor foaming and viscosity behavior. They are often used in combination with alkyl ether sulfates and amphoteric surfactants.

Sulfosuccinic esters (sulfosuccinates) are mild and readily foaming surfactants, but, due to their poor ability to be thickened, are preferably used only together with other anionic and amphoteric surfactants and, because of their low hydrolysis stability, preferably only in neutral or well-buffered products.

Amidopropylbetaines have excellent skin and eye mucosa compatibility. In combination with anionic surfactants, their mildness can be synergistically improved. The use of cocamidopropylbetaine is preferred.

Amphoacetates/amphodiacetates as amphoteric surfactants have very good skin and mucosa compatibility and can have a conditioning effect and increase the care effect of additives. They are used in a similar way to the betaines for optimizing alkyl ether sulfate formulations. Sodium cocoamphoacetate and disodium cocoamphodiacetate are most preferred.

Alkyl polyglycosides are mild, have good universal properties, but are weakly foaming. For this reason, they are preferably used in combinations with anionic surfactants.

Polysorbates

In addition, according to the invention, polysorbate agents can advantageously be incorporated into the composition.

Polysorbates advantageous for the purposes of the invention here are
polyoxyethylene(20) sorbitan monolaurate (Tween 20, CAS No. 9005-64-5)
polyoxyethylene(4) sorbitan monolaurate (Tween 21, CAS No. 9005-64-5)
polyoxyethylene(4) sorbitan monostearate (Tween 61, CAS No. 9005-67-8)
polyoxyethylene(20) sorbitan tristearate (Tween 65, CAS No. 9005-71-4)
polyoxyethylene(20) sorbitan monooleate (Tween 80, CAS No. 9005-65-6)
polyoxyethylene(5) sorbitan monooleate (Tween 81, CAS No. 9005-65-5)
polyoxyethylene(20) sorbitan trioleate (Tween 85, CAS No. 9005-70-3).

Of particular advantage are, in particular,
polyoxyethylene(20) sorbitan monopalmitate (Tween 40, CAS No. 9005-66-7)
polyoxyethylene(20) sorbitan monostearate (Tween 60, CAS No. 9005-67-8).

According to the invention, these are advantageously used in a concentration of from 0.1 to 5% by weight and in particular in a concentration of from 1.5 to 2.5% by weight, based on the total weight of the composition individually or as a mixture of two or more polysorbates.

In one embodiment of the invention, the compositions according to the invention comprise copolymer a) and, in each case based on the composition, less than 1% by weight of, preferably less than 0.1% by weight of and in particular no oligomer b).

The cosmetic compositions according to the invention comprising copolymer a) can be used advantageously for removing excess oil or lipid from the surface of the skin. In particular, these compositions comprise, based on the composition, less than 1% by weight of, preferably, less than 0.1% by weight of and in particular no oligomer b).

Self-Tanning Products

Standard commercial self-tanning products are generally O/W emulsions. In these, the water phase is stabilized by emulsifiers customary in cosmetics. The required additional stabilization through carbomers is disadvantageous. Their use in conjunction with self-tanning agents, in particular with dihydroxyacetone (DHA), leads, as a result of chemical reaction, to a yellowish discoloration of the preparation and to odor impairments. One alternative to using carbomers is to use xanthan gum. Although this gives stable products, an unpleasant sticky feel on the skin often has to be accepted.

The compositions according to the invention can be present and used in various forms. Thus they may, for example, an emulsion of the oil-in-water (O/W) type or a multiple emulsion, for example of the water-in-oil-in-water (W/O/W) type. Emulsifier-free formulations, such as hydrodispersions, hydrogels or a Pickering emulsion are also advantageous embodiments.

The consistency of the formulations can range from pasty formulations via flowable formulations to low viscosity sprayable products. Accordingly, creams, lotions or sprays can be formulated. For use, the cosmetic compositions according to the invention are applied to the skin in the manner customary for cosmetics and dermatological products in a sufficient amount.

Through the use it is possible not only to achieve an even skin color, it is also possible to color areas of skin which are differently colored as a result of nature or as a result of pathological change.

The self-tanning agents used advantageously according to the invention are, inter alia, glycerol aldehyde, hydroxymethylglyoxal, γ-dialdehyde, erythrulose, 5-hydroxy-1,4-naphthoquinone (juglone), and 2-hydroxy-1,4-naphthoquinone which occurs in henna leaves.

For the purposes of the invention, very particular preference is given to 1,3-dihydroxy-acetone (DHA), a trivalent sugar which occurs in the human body. 6-Aldo-D-fructose and ninhydrin can also be used as self-tanning agents according to the invention. For the purposes of the invention, self-tanning agents are also understood as meaning substances which bring about a skin color which departs from a brown shade.

In a preferred embodiment of the invention, these compositions comprise two or more self-tanning substances in a concentration of from 0.1 to 10% by weight and particularly preferably from 0.5 to 6% by weight, in each case based on the total weight of the composition.

Preferably, these compositions comprise 1,3-dihydroxyacetone as self-tanning substance. Further preferably, these compositions comprise organic and/or inorganic photoprotective filters. The compositions can also comprise inorganic and/or organic and/or modified inorganic pigments.

Customary and advantageous ingredients also present in the compositions according to the invention are specified above and in DE 103 21 147, [0024] to [0132].

The invention also relates to the cosmetic use of such compositions for coloring the skin of multicellular organisms, in particular the skin of humans and animals, in particular also for evening out the color of areas of skin that are pigmented to differing degrees.

A further advantage of the preparations thickened by means of the W/W emulsion polymers is that O/W and W/O/W emulsions with a high fraction of humectants, such as, for example, glycerol, can be stably provided.

A further advantage of using the W/W emulsion polymers for modifying the rheology of cosmetic or dermatological preparations is that a thickening effect is achieved over a broad pH range from 4 to 11 and in particular from 6 to 10. In contrast to standard commercial thickeners, it is possible to achieve a continuous increase in the viscosity whereas standard commercial thickeners in most cases only permit a stepped change in the viscosity in relatively large steps. Consequently, it is more easily possible to establish viscosities as are suitable, for example, for body milks or lotions.

The examples below illustrate the invention without, however, limiting it thereto.

### EXAMPLES

The K values of the polymers were determined according to H. Fikentscher, Cellulose-Chemie [Cellulose Chemistry], volume 13, 58-64 and 71-74 (1932) in 3% strength by weight aqueous sodium chloride solution at 25° C., a concentration of 0.1% by weight.

The viscosity of the dispersions was measured in each case in a Brookfield viscosimeter with a spindle No. 4 at 20 rpm and a temperature of 20° C. Unless stated otherwise, the data are in % by weight.

The polymers of groups a) and b) used as stabilizers in the examples had the following composition:
Stabilizer 1: graft polymer of vinyl acetate on polyethylene glycol of molecular weight M_{N} 6000, polymer concentration 20%
Stabilizer 2: hydrolyzed copolymer of vinyl methyl ether and maleic acid in the form of the free carboxyl groups, polymer concentration 35%
Stabilizer 3: copolymer of methyl polyethylene glycol methacrylate and methacrylic acid of molar mass M_{w} 1500, polymer concentration 40%
Stabilizer 4: polypropylene glycol with a molecular weight M_{N} of 600
Stabilizer 5: polypropylene glycol with a molecular weight M_{N} of 900
Stabilizer 6: polypropylene glycol with a molecular weight M_{N} of 1000 and terminally capped at one end with a methyl group
Stabilizer 7: block copolymer of polyalkylene glycols with a molecular weight M_{N} of 1000
Stabilizer 8: maltodextrin (C-PUR01910, 100% strength)
Stabilizer 9: polypropylene glycol with a molecular weight M_{N} of 2000 and terminally capped at one end with a methyl group
Stabilizer 10: copolymer of acrylamide and DMAEMA quaternized with a molecular weight M_{N} of 500-6000
Stabilizer 11: copolymer of methacrylic acid and acrylamidomethylpropanesulfonic acid with a molar ratio of 80:20
Stabilizer 12: copolymer of methacrylic acid and acrylamidomethylpropanesulfonic acid with a molar ratio of 70:30
Stabilizer 13: copolymer of methacrylic acid and acrylamidomethylpropanesulfonic acid with a molar ratio of 60:40
Stabilizer 14: Pluronic® PE 4300: block copolymer von ethylene oxide and propylene oxide of the general formula (I) (see above), where the mass of the polypropylene glycol block is about 1100 g/mol and about 30% by weight of polyethylene glycol are present per molecule of the block copolymer.
Stabilizer 15: Pluronic® PE 6200: block copolymer of ethylene oxide and propylene oxide of the general formula (I) (see above), where the mass of the polypropylene glycol block is about 1750 g/mol and about 20% by weight of polyethylene glycol are present per molecule of the block copolymer.
Stabilizer 16: copolymer of 50 mol % of acrylamide and 50 mol % of dimethylaminoethyl acrylate methochloride, K value 82.6;
Stabilizer 17: copolymer of 50 mol % of acrylamide and 45 mol % of dimethylaminoethyl acrylate methochloride, 5 mol % of acrylic acid, K value 45.1
Stabilizer 18: copolymer of 60 mol % of acrylamide and 38 mol % of dimethylaminoethyl acrylate methochloride, 2 mol % of acrylic acid, K value 78.0.

In the examples, the following polymerization initiators were used:

Azostarter VA-044®: 2,2′-azobis(N,N′-dimethyleneisobutyramidine) dihydrochloride

Azostarter V-70®: 2,2′-azobis(4-methoxy-2,4-dimethylvaleronitrile)

Azostarter V-65®: 2,2′-azobis(2,4-dimethylvaleronitrile)

### Example 1

In a 250 ml-capacity four-necked flask which was equipped with a Teflon stirrer and a device for working under nitrogen, while passing nitrogen through,

90.0 g of stabilizer 1,

51.4 g of stabilizer 2 and

28.6 g of completely demineralized water

were initially introduced and stirred at a speed of 300 rpm. 30 g of acrylic acid were added dropwise to this solution over the course of 5 to 10 minutes, the mixture was heated to 50° C., 0.03 g of 2,2′-azobis(N,N′-dimethyleneisobutyramidine) dihydrochloride (Azostarter VA-044®) was added and the mixture was polymerized for 5 hours at 50° C. The reaction mixture was then treated with 0.05 g of Azostarter VA-044® and after-polymerized for 1 hour at 60° C. This gave an aqueous dispersion with a solids content of 33%. It had a pH of 4 and a viscosity of 5950 mPas. The polymer had a K value of 120.7. By adding water to the dispersion, a 2% strength aqueous solution was produced which, at a pH of 7, had a viscosity of 2640 mPas. The particle size distribution of the dispersed particles of the polymer dispersion was 3 to 8 μm.

### Example 2

In the device stated in Example 1,

90.0 g of stabilizer 1,

51.4 g of stabilizer 2 and

28.6 g of completely demineralized water

were initially introduced and, while passing nitrogen through, stirred at a speed of 300 rpm. A mixture of 30 g of acrylic acid and 0.09 g of triallylamine as crosslinker was added dropwise to this solution over the course of 5 to 10 minutes, and the mixture was heated to a temperature of 40° C. over the course of 5 to 10 minutes. 0.03 g of 2,2′-azobis(4-methoxy-2,4-dimethylvaleronitrile) (Azostarter V-70®) was then added and the mixture was polymerized for 5 hours at a temperature of 40° C. Then, for the after-polymerization, 0.05 g of Azostarter V-70® was added and the dispersion was heated to a temperature of 50° C. for one hour. This gave an aqueous dispersion with a viscosity of 2700 mPas. It had a pH of 4. By adding water to the aqueous dispersion, a 2% strength aqueous solution was produced. It had a viscosity of 39000 mPas at pH 7. The particle size distribution of the dispersed particles of the polymer dispersion was 5 to60μm.

### Example 3

Example 2 was repeated except that in the polymerization apparatus

12 g of stabilizer 4

51.4 g of stabilizer 2 and

106.6 g of completely demineralized water

were initially introduced and the use of triallylamine was dispensed with. This gave an aqueous emulsion which, at a pH of 4, had a viscosity of 2240 mPas.

### Example 4

In the apparatus given in Example 1,

1.5 g of stabilizer 5

16.5 g of stabilizer 4

18.0 g of stabilizer 8 and

104.0 g of completely demineralized water

were initially introduced, the mixture was stirred continuously at 300 rpm, and 30 g of acrylic acid were added continuously over the course of 5 to 10 minutes. The pH of the reaction mixture was then adjusted from 4.5 to 3 by adding 30 g of 32% strength hydrochloric acid, and the emulsion was heated to a temperature of 50° C. After adding 0.03 g of Azostarter VA-044®, the emulsion was polymerized for 5 hours at 50° C., then 0.05 g of Azostarter VA-044 was added and the mixture was after-polymerized for a further 1 hour at 50° C. This gave an aqueous dispersion with a viscosity of 208 mPas.

### Example 5

Example 1 was repeated except that in the polymerization apparatus a mixture of

45 g of stabilizer 3

51.4 g of stabilizer 2 and

73.6 g of completely demineralized water

was initially introduced. This gave an aqueous emulsion with a viscosity of 3650 mPas. The particle size distribution of the dispersed particles of the polymer dispersion was 3 to 10 μm.

### Example 6

In the device given in Example 1,

90.0 g of stabilizer 1,

51.4 g of stabilizer 2 and

28.6 g of completely demineralized water

were initially introduced and, while passing nitrogen through, stirred at a speed of 300 rpm. A mixture of 30 g of acrylic acid and 0.22 g of pentaerythritol triallyl ether (70% strength) as crosslinker was added dropwise to this solution over the course of 5 to 10 minutes, and the mixture was heated to a temperature of 40° C. over the course of 5 to 10 minutes. 0.03 g of Azostarter V-70® was then added and the mixture was polymerized for 5 hours at a temperature of 40° C. Then, for the after-polymerization, 0.05 g of Azostarter VA-044® was added, and the dispersion was heated to a temperature of 50° C. for one hour. This gave an aqueous dispersion with a viscosity of 2900 mPas. By adding water and adjusting the pH to 7, a 2% strength aqueous solution was produced which had a viscosity of 10 000 mPas. The particle size distribution of the dispersed particles of the polymer dispersion was 5 to 70 μm.

### Example 7

In a 250 ml-capacity four-necked flask which was equipped with a Teflon stirrer and a device for working under nitrogen, while passing nitrogen through,

90.0 g of stabilizer 1,

18.0 g of stabilizer 8 and

62.0 g of completely demineralized water

were initially introduced and stirred at a speed of 200 rpm. 30 g of acrylic acid were added dropwise to this solution over the course of 5 to 10 minutes, the mixture was heated to 50° C., 0.03 g of Azostarter VA-044® was added and the mixture was polymerized at 50° C. for 5 hours. The reaction mixture was then treated with 0.05 g of Azostarter VA-044® and after-polymerized for 1 hour at 60° C. This gave an aqueous dispersion with a solids content of 33%. It had a pH of 2 and a viscosity of 10500 mPas. A 2% strength solution prepared therefrom by adding water had, at a pH of 7, a viscosity of 2000 mPas. The particle size distribution of the dispersed particles of the polymer dispersion was 5 to 40 μm.

### Example 8

In the device stated in Example 1

90.0 g of stabilizer 1,

51.4 g of stabilizer 2 and

28.6 g of completely demineralized water

were initially introduced and, while passing nitrogen through, stirred at a speed of 300 rpm. A mixture of 30 g of acrylic acid and 0.09 g of triallylamine as crosslinker was added dropwise to this solution over the course of 5 to 10 minutes, and the emulsion was heated to a temperature of 50° C. over the course of 5 to 10 minutes. 0.03 g of Azostarter V-65® was then added, and the mixture was polymerized for 5 hours at a temperature of 50° C. Then, for the after-polymerization, 0.05 g of Azostarter VA-044® was added, and the dispersion was heated to a temperature of 60° C. for one hour. This gave an aqueous dispersion with a viscosity of 3700 mPas. It had a pH of 4. By adding water to the aqueous dispersion, a 2% strength aqueous solution was produced. It had a viscosity of 29 000 mPas at pH 7. The particle size distribution of the dispersed particles of the polymer dispersion was 5 to 30 μm.

### Example 9

In the device stated in Example 1,

90.0 g of stabilizer 1,

45.7 g of stabilizer 2 and

34.3 g of completely demineralized water

were initially introduced and, while passing nitrogen through, stirred at a speed of 300 rpm. A mixture of 30 g of acrylic acid and 0.09 g of triallylamine as crosslinker was added dropwise to this solution over the course of 5 to 10 minutes, and the mixture was heated to a temperature of 40° C. over the course of 5 to 10 minutes. 0.03 g of Azostarter V-70® was then added and the mixture was polymerized for 5 hours at a temperature of 40° C. For the after-polymerization, 0.05 g of Azostarter VA-044® was added and the dispersion was heated to a temperature of 50° C. for one hour. This gave an aqueous dispersion with a viscosity of 2300 mPas. By adding water and adjusting the pH to 7, a 2% strength aqueous solution was produced which had a viscosity of 32 000 mPas.

### Example 10

In the apparatus stated in Example 1,

18.0 g of stabilizer 9

18.0 g of stabilizer 8 and

90.0 g of completely demineralized water

were initially introduced, the mixture was stirred continuously at 300 rpm while passing nitrogen through, and 30 g of acrylic acid were added continuously over the course of 5 to 10 minutes. The pH of the reaction mixture was then adjusted from 4.5 to 3 by adding 30 g of 32% strength hydrochloric acid, and the emulsion was heated to a temperature of 50° C. After adding 0.03 g of Azostarter VA-044®, the emulsion was polymerized at 50° C. for 5 hours, then 0.05 g of Azostarter VA-044® was added, and the mixture was polymerized for a further 1 hour at 50° C. This gave an aqueous dispersion with a viscosity of 320 mPas.

### Example 11

In thedevicestated in Example 1,

63.0 g of stabilizer 7

9.0 g of stabilizer 8

400 g of water and

45 g of acrylic acid

were initially introduced and stirred at a speed of 100 rpm while passing nitrogen through. 0.45 g of sodium persulfate and 14.4 g of water were added to this solution, and initial polymerization was carried out for 15 minutes at 25° C. Then, 135 g of acrylic acid and 27 g of stabilizer 8 were introduced over 2 hours at 25° C. At the same time, 0.18 g of ascorbic acid was introduced over 7 hours. The mixture was then after-polymerized for a further hour. This gave an aqueous dispersion with a viscosity of 800 mPas and a pH of 1.5. By adding water and sodium hydroxide solution, a 2% strength dispersion with a pH of 7 was produced. The viscosity of the dispersion was 5000 mPas.

### Example 12

In a 2 liter-capacity four-necked flask which was equipped with a Teflon stirrer and a device for working under nitrogen, while passing nitrogen through,

257.0 g of stabilizer 1,

449.0 g of stabilizer 2 and

102.5 g deionized water

were initially introduced and, while passing nitrogen through, stirred at a speed of 200 rpm for 10 minutes. 60 g of acrylic acid were added dropwise to this solution over 10 min, the reaction mixture was heated to 60° C. and a solution of 90 g of acrylic acid and 1.5 g of ethoxylated trimethylenepropane triacrylate was added over the course of 3.5 hours. At the same time as the addition of the acrylic acid/trimethylolpropane triacrylate solution, the four-hour addition of a solution of 0.15 g of Azostarter VA-044® in 40 g of water was started. After the end of the addition, the mixture was further stirred for 30 min at 60° C. Finally, a further 0.225 g of VA-044® was added and the polymerization was continued for a further hour at 60° C. After cooling to room temperature, an aqueous dispersion with a polymer content of 15% by weight, a viscosity of 5350 mpa*s and a pH of 4.5 was obtained. By adding appropriate amounts of water and sodium hydroxide solution, a dispersion with a solids content of 2% by weight, a pH of 7 and a viscosity of 10 900 mpa*s was produced.

### Example 13

In the apparatus from Example 12, while passing nitrogen through,

257.0 g of stabilizer 1,

449.0 g of stabilizer 2 and

102.5 g of deionized water

were initially introduced and, while passing nitrogen through, stirred at a speed of 200 rpm for 10 minutes. 60 g of acrylic acid and 0.015 g of VA-044® were added dropwise to this solution over the course of 10 min, the reaction mixture was heated to 60° C., and a solution of 90 g of acrylic acid and 1.5 g of ethoxylated trimethylolpropane triacrylate was added over the course of 3.5 hours. At the same time as the addition of the acrylic acid/trimethylenepropane triacrylate solution, the four-hour addition of a solution of 0.135 g of Azostarter VA-044® in 40 g of water was started. After the end of the addition, the mixture was further stirred for 30 min at 60° C. Finally, a further 0.225 g of VA-044® was added and the polymerization was continued for a further hour at 60° C. After cooling to room temperature, an aqueous dispersion with a polymer content of 15% by weight, a viscosity of 5550 mpa*s and a pH of 4.5 was obtained. By adding appropriate amounts of water and sodium hydroxide solution, a dispersion with a solids content of 2% by weight, a pH of 7 and a viscosity of 10 300 mpa*s was produced.

### Example 14

In the apparatus from Example 12, while passing nitrogen through,

257.0 g of stabilizer 1,

449.0 g of stabilizer 2 and

102.5 g of deionized water

were introduced and, while passing nitrogen through, stirred at a speed of 200 rpm for 10 minutes. 60 g of acrylic acid and 0.015 g of VA-044® were added dropwise to this solution over the course of 10 minutes, the reaction mixture was heated to 60° C., and a solution of 90 g of acrylic acid and 1.5 g of triallylamine was added over the course of 3.5 hours. At the same time as the addition of the acrylic acid/triallylamine solution, the four-hour addition of a solution of 0.135 g of Azostarter VA-044® in 40 g of water was started. After the end of the addition, the mixture was further stirred for 30 min at 60° C. Finally, a further 0.225 g of VA-044® was added and the polymerization was continued for a further hour at 60° C. After cooling to room temperature, an aqueous dispersion with a polymer content of 15% by weight, a viscosity of 10 250 mpa*s and a pH of 4.5 was obtained. By adding appropriate amounts of water and sodium hydroxide solution, a dispersion with a solids content of 2% by weight, a pH of 7 and a viscosity of 28 500 mpa*s was produced.

### Example 15

In the apparatus from Example 12, while passing nitrogen through,

257.0 g of stabilizer 1,

449.0 g of stabilizer 2 und

102.5 g of deionized water

were initially introduced and, while passing nitrogen through, stirred at a speed of 200 rpm for 10 minutes. 60 g of acrylic acid and 0.015 g of VA-044® were added dropwise to this solution over the course of 10 min, the reaction mixture was heated to 60° C. and a solution of 75 g of acrylic acid, 15 g of methyl methacrylate and 1.5 g of triallylamine was added over the course of 3.5 hours. At the same time as the addition of the acrylic acid/triallylamine solution, the four-hour addition of a solution of 0.135 g of Azostarter VA-044® in 40 g of water was started. After the end of the addition, the mixture was further stirred for 30 min at 60° C. Finally, a further 0.225 g of VA-044® was added and the polymerization was continued for a further hour at 60° C. After cooling to room temperature, an aqueous dispersion with a polymer content of 15% by weight, a viscosity of 5800 mpa*s and a pH of 4.5 was obtained. By adding appropriate amounts of water and sodium hydroxide solution, a dispersion with a solids content of 2% by weight, a pH of 7 and a viscosity of 17 500 mpa*s was produced.

### Example 16

In the apparatus from Example 12, while passing nitrogen through,

257.0 g of stabilizer 1,

449.0 g of stabilizer 2 and

102.5 g of deionized water

were initially introduced and, while passing nitrogen through, stirred at a speed of 200 rpm for 10 minutes. 60 g of acrylic acid and 0.015 g of VA-044® were added dropwise to this solution over the course of 10 min, the reaction mixture was heated to 60° C. and a solution of 82.5 g of acrylic acid, 7.5 g of methyl methacrylate and 1.5 g of triallylamine was added over the course of 3.5 hours. At the same time as the addition of the acrylic acid/triallylamine solution, the four-hour addition of a solution of 0.135 g of Azostarter VA-044® in 40 g of water was started. After the end of the addition, the mixture was further stirred for 30 min at 60° C. Finally, a further 0.225 g of VA-044® was added and the polymerization was continued for a further hour at 60° C. After cooling to room temperature, an aqueous dispersion with a polymer content of 15% by weight, a viscosity of 21 900 mpa*s and a pH of 4.5 was obtained. By adding appropriate amounts of water and sodium hydroxide solution, a dispersion with a solids content of 2% by weight, a pH of 7 and a viscosity of 23 650 mpa*s was produced.

### Example 17

In a 250 ml capacity four-necked flask which was equipped with a Teflon stirrer and a device for working under nitrogen, while passing nitrogen through,

87.5 g (8.75%) of stabilizer 14,

87.5 g (8.75%) of stabilizer 15,

25 g (2.5%) of stabilizer 11 with a K value of 74.2,

442.8 g of completely demineralized water

were initially introduced and stirred at a speed of 200 rpm. A mixture of 174 g of acrylic acid and 1.134 g of pentaerythritol triallyl ether was added dropwise to this solution over the course of 5 to 10 minutes, the mixture was heated to 40° C., 0.2 g of Azostarter VA-044®, which was dissolved in 20 g of water, was added, and the mixture was polymerized for 4 hours at 40° C. The reaction mixture was then treated with 0.3 g of Azostarter VA-044® in 20 g of water and after-polymerized for 1 hour at 40° C. This gave an aqueous dispersion with a solids content of 37.5 %.

### Example 18

In a 250 ml capacity four-necked flask equipped with a Teflon stirrer and a device for working under nitrogen, while passing nitrogen through,

87.5 g (8.75%) of stabilizer 14,

87.5 g (8.75%) of stabilizer 15,

20 g (2%) of stabilizer 12 with a K value of 92.3,

409.0 g of completely demineralized water

were initially introduced and stirred at a speed of 200 rpm. A mixture of 174 g of acrylic acid and 1.05 g of pentaerythritol triallyl ether was added dropwise to this solution over the course of 5 to 10 minutes, the mixture was heated to 40° C., 0.2 g of Azostarter VA-044®, which was dissolved in 20 g of water, was added, and the mixture was polymerized for 4 hours at 40° C. The reaction mixture was then treated with 0.3 g of Azostarter VA-044® in 20 g of water and after-polymerized for 1 hour at 40° C. This gave an aqueous dispersion with a solids content of 37.1%.

### Example 19

In a 250 ml capacity four-necked flask which was equipped with a Teflon stirrer and a device for working under nitrogen, while passing nitrogen through,

87.5 g (8.75%) of stabilizer 14,

87.5 g (8.75%) of stabilizer 15,

20 g (2%) of stabilizer 13 with a K value of 84.1,

359.5 g of completely demineralized water

were initially introduced and stirred at a speed of 200 rpm. A mixture of 174 g of acrylic acid and 1.05 g of pentaerythritol triallyl ether was added dropwise to this solution over the course of 5 to 10 minutes, the mixture was heated to 40° C., 0.2 g of Azostarter VA-044®, which was dissolved in 20 g of water, was added, and the mixture was polymerized for 4 hours at 40° C. The reaction mixture was then treated with 0.3 g of Azostarter VA-044® in 20 g of water and after-polymerized for 1 hour at 40° C. This gave an aqueous dispersion with a solids content of 37.5%.

### Example 20

In a 250 ml capacity four-necked flask which was equipped with a Teflon stirrer and a device for working under nitrogen, while passing nitrogen through,

87.5 g (8.75%) of stabilizer 14,

87.5 g (8.75%) of stabilizer 15,

24 g (2.4%) of stabilizer 11,

398.13 g of completely demineralized water

were initially introduced and stirred at a speed of 200 rpm. A mixture of 172.3 g of acrylic acid, 1.74 g of octadecyl vinyl ether and 1.05 g of pentaerythritol triallyl ether was added dropwise to this solution over the course of 5 to 10 minutes, the mixture was heated to 40° C., 0.2 g of Azostarter VA-044®, which was dissolved in 20 g of water, was added, and the mixture was polymerized for 5 hours at 40° C. The reaction mixture was then treated with 0.3 g of Azostarter VA-044® in 20 g of water and after-polymerized for 1 hour at 40° C. This gave an aqueous dispersion with a solids content of 37.5%.

### Example 21

In a 250 ml capacity four-necked flask which was equipped with a Teflon stirrer and a device for working under nitrogen, while passing nitrogen through,

87.5 g (8.75%) of stabilizer 14,

87.5 g (8.75%) of stabilizer 15,

24 g (2.4%) of stabilizer 12,

509.0 of completely demineralized water

were initially introduced and stirred at a speed of 200 rpm. A mixture of 165.3 g of acrylic acid, 17.4 g of hexene-1 and 1.05 g of pentaerythritol triallyl ether was added dropwise to this solution over the course of 5 to 10 minutes, the mixture was heated to 40° C., 0.2 g of Azostarter VA-044®, which was dissolved in 20 g of water, was added, and the mixture was polymerized for 5 hours at 40° C. The reaction mixture was then treated with 0.3 g of Azostarter VA-044® in 20 g of water and after-polymerized for 1 hour at 40° C. This gave an aqueous dispersion with a solids content of 37.5%.

### Example 22

In a glass reactor which was equipped with an anchor stirrer and a device for working under nitrogen, while passing nitrogen through and with constant stirring at a speed of 200 rpm, 489 g of completely demineralized water, 175 g of a block copolymer of ethylene oxide (EO) and propylene oxide (PO) with a content of EO of 20% and a molar mass of 1750 g/mol of the polypropylene glycol block and 120 g of a 20% strength aqueous solution of a copolymer of 59 parts by weight of acrylamidomethylpropanesulfonic acid, 20 parts by weight of methyl acrylate, 20 parts by weight of acrylic acid and 1 part by weight of styrene were initially introduced. Then, with stirring (200 rpm), a mixture of 174 g of acrylic acid and 1.5 g of pentaerythritol triallyl ether (70% strength) was added dropwise over the course of 5 minutes and the resulting emulsion was heated to 40° C. After adding a solution of 0.2 g of Azostarter VA-044 in 10 g of water and rinsing the metering device with 10 g of water, the reaction mixture was heated to a temperature of 40° C. and held at this temperature for 4 hours. Then, a solution of 0.3 g of Azostarter VA-044 in 10 g of completely demineralized water was metered in, then rinsed with 10 g of completely demineralized water and the reaction mixture was then stirred for a further 1 hour for the afterpolymerization at 40° C. This gave a milky white dispersion with a viscosity of 18 800 mPas. A 0.5% strength aqueous solution had a viscosity of 26 600 mPas at pH 7.

### Example 23

In a glass reactor which was equipped with an anchor stirrer and a device for working under nitrogen, while passing nitrogen through and with constant stirring at a speed of 200 rpm, 489 g of completely demineralized water, 175 g of a copolymer of 20.3 parts by weight of propylene oxide and 14.2 parts by weight of ethylene oxide and 120 g of a 20% strength aqueous solution of a copolymer of 59 parts by weight of acrylamidomethylpropanesulfonic acid, 20 parts by weight of methyl acrylate, 20 parts by weight of acrylic acid and 1 part by weight of styrene were initially introduced. Then, with stirring (200 rpm), a mixture of 174 g of acrylic acid and 1.5 g of pentaerythritol triallyl ether (70% strength) was added dropwise over the course of 5 minutes and the resulting emulsion was heated to 40° C. After adding a solution of 0.2 g of Azostarter VA-044 in 10 g of water and rinsing with 10 g of water, the reaction mixture is heated to a temperature of 40° C. and polymerized at this temperature for 4 hours. Then, a solution of 0.3 g of Azostarter VA-044 in 10 g of completely demineralized water was metered in, rinsed with 10 g of completely demineralized water, and the reaction mixture was then stirred for a further 1 hour for the after-polymerization at 40° C. This gave a milky white dispersion with a viscosity of 19 600 mPas. A 0.5% strength aqueous solution had a viscosity of 22 400 mPas at pH 7.

### Example 24

Example 22 was repeated except that in the polymerization reactor 359 g of completely demineralized water, 87.5 g of a block copolymer of ethylene oxide (EO) and propylene oxide (PO) with a content of EO of 20% and a molar mass of 1750 g/mol of the polypropylene glycol block, 87.5 g of a copolymer of 20.3 parts by weight of propylene oxide and 14.2 parts by weight of ethylene oxide, and 250 g of a 10% strength aqueous solution of a copolymer of 90 mol % of methacrylic acid and 10 mol % of the Na salt of acrylamidomethylpropanesulfonic acid were initially introduced. This gave a white dispersion with a viscosity of 1000 mPas. A 0.5% strength aqueous solution prepared therefrom had a viscosity of 30 000 mPas at a pH of 7.

### Example 25

Example 24 was repeated with the sole exception that instead of the copolymer of 90 mol % of methacrylic acid and 10 mol % of the Na salt of acrylamidomethylpropanesulfonic acid, now a copolymer of the composition 10 mol % of methacrylic acid and 90 mol % of the Na salt of acrylamidomethylpropanesulfonic acid was used. This gave an aqueous dispersion with a viscosity of 1500 mPas. An aqueous solution of the dispersion diluted to 0.5% had a viscosity of 25 000 mPas at a pH of 7.

### Example 26

Example 24 was repeated with the sole exception that instead of the copolymer of 90 mol % of methacrylic acid and 10 mol % of the Na salt of acrylamidomethylpropane-sulfonic acid, now a copolymer of the composition 50 mol % of methacrylic acid and 50 mol % of the Na salt of acrylamidomethylpropanesulfonic acid was used. This gave an aqueous dispersion with a viscosity of 1200 mPas. An aqueous solution of the dispersion diluted to 0.5% had a viscosity of 35 000 mPas at a pH of 7.

### Example 27

Example 24 was repeated with the sole exception that instead of the copolymer of 90 mol % of methacrylic acid and 10 mol % of the Na salt of acrylamidomethylpropane-sulfonic acid, now a copolymer of the composition 80 mol % of methacrylic acid and 20 mol % of the Na salt of acrylamidomethylpropanesulfonic acid was used. This gave an aqueous dispersion with a viscosity of 1300 mPas. An aqueous solution of the dispersion diluted to 0.5% had a viscosity of 33 000 mPas at a pH of 7.

### Example 28

Example 24 was repeated with the sole exception that instead of the copolymer of 90 mol % of methacrylic acid and 10 mol % of the Na salt of acrylamidomethylpropane-sulfonic acid, now a copolymer of the composition 70 mol % of methacrylic acid and 30 mol % of the Na salt of acrylamidomethylpropanesulfonic acid was used. This gave an aqueous dispersion with a viscosity of 1100 mPas. An aqueous solution of the dispersion diluted to 0.5% had a viscosity of 29 000 mPas at a pH of 7.

### Example 29

In a glass reactor which was equipped with an anchor stirrer and a device for working under nitrogen, while passing nitrogen through and with continuous stirring at a speed of 200 rpm, 359 g of completely demineralized water, 87.5 g of a block copolymer of ethylene oxide (EO) and propylene oxide (PO) with a content of EO of 20% and a molar mass of 1750 g/mol of the polypropylene glycol block, 87.5 g of a copolymer of 20.3 parts by weight of propylene oxide and 14.2 parts by weight of ethylene oxide and 250 g of a 10% strength aqueous solution of a copolymer of 80 mol % of methacrylic acid and 20 mol % of the Na salt of acrylamidomethylpropanesulfonic acid were initially introduced.

Then, with stirring (200 rpm), a mixture of 174 g of acrylic acid and 1.5 g of pentaerythritrol triallyl ether (70% strength) was added dropwise over the course of 5 minutes and the resulting emulsion was heated to 50° C. After adding a solution of 0.2 g of Azostarter VA-044 in 10 g of water and rinsing the metering device with 10 g of water, the reaction mixture was heated to a temperature of 50° C. and polymerized at this temperature for 4 hours. Then, a solution of 0.3 g of Azostarter VA-044 in 10 g of completely demineralized water was metered in, the metering device was rinsed with 10 g of completely demineralized water and the reaction mixture was then stirred for a further 1 hour for the afterpolymerization at 50° C. This gave a milky white dispersion with a viscosity of 1600 mPas. A 0.5% strength aqueous solution of this dispersion had a viscosity of 29 000 mPas at pH 7.

### Example 30

In a glass reactor which was equipped with an anchor stirrer and a device for working under nitrogen, while passing nitrogen through and with constant stirring at a speed of 200 rpm, 359 g of completely demineralized water, 87.5 g of a block copolymer of ethylene oxide (EO) and propylene oxide (PO) with a content of EO of 20% and a molar mass of 1750 g/mol of the polypropylene glycol block, 87.5 g of a copolymer of 20.3 parts by weight of propylene oxide and 14.2 parts by weight of ethylene oxide and 250 g of a 10% strength aqueous solution of a copolymer of 80 mol % of methacrylic acid and 20 mol % of the Na salt of acrylamidomethylpropanesulfonic acid.

Then, with stirring (200 rpm), a mixture of 174 g of acrylic acid and 1.5 g of pentaerythritol triallyl ether (70% strength) was added dropwise over the course of 5 minutes and the resulting emulsion was heated to 35° C. After adding a solution of 0.2 g of Azostarter V-70 in 10 g of water and rinsing the metering device with 10 g of water, the reaction mixture was heated to a temperature of 50° C. and polymerized at this temperature for 4 hours. Then, a solution of 0.3 g of Azostarter V-70 in 10 g of completely demineralized water was metered in, the metering device was rinsed with 10 g of completely demineralized water and the reaction mixture was then stirred for a further 1 hour for the afterpolymerization at 35° C. This gave a milky white dispersion with a viscosity of 1400 mPas. A 0.5% strength aqueous solution of this dispersion had a viscosity of 32 000 mPas at pH 7.

### Example 31

Example 22 was repeated except that a solution of 87.5 g of a block copolymer of ethylene oxide (EO) and propylene oxide (PO) with a content of EO of 20% and a molar mass of 1750 g/mol of the polypropylene glycol block, 87.5 g of a copolymer of 20.3 parts by weight of propylene oxide and 14.2 parts by weight of ethylene oxide, 53.4 g of a 45% strength aqueous polyacrylic acid of molar mass M_{w} 50 000 and 555.7 g of completely demineralized water was initially introduced. This gave an aqueous dispersion with a viscosity of 2000 mPas. A 0.5% strength aqueous solution of this dispersion had a viscosity of 26 000 mPas at pH 7.

### Example 32

Example 22 was repeated except that a solution of 87.5 g of a block copolymer of ethylene oxide (EO) and propylene oxide (PO) with a content of EO of 20% and a molar mass of 1750 g/mol of the polypropylene glycol block, 87.5 g of a copolymer of 20.3 parts by weight of propylene oxide and 14.2 parts by weight of ethylene oxide, 120 g of a 20% strength aqueous solution of polyacrylamidomethylpropanesulfonic acid and 555.7 g of completely demineralized water was initially introduced. This gave an aqueous dispersion with a viscosity of 1900 mPas. A 0.5% strength aqueous solution of this dispersion had a viscosity of 28 000 mPas at pH 7.

### Example 33

Example 32 was repeated with the sole exception that in the initial charge, the polyacrylamidomethylpropanesulfonic acid was replaced by the same amount of 20% strength aqueous polymethacrylic acid of molar mass M_{w} 40 000. This gave an aqueous dispersion with a viscosity of 1900 mPas. A 0.5% strength aqueous solution of this dispersion had a viscosity of 36 000 mPas at pH 7.

### Example 34

Example 27 was repeated with the sole exception that instead of pentaerythritol triallyl ether, now 1.75 g of a 10% strength aqueous solution of an ethoxylated trimethylolpropane triacrylate was used as crosslinker. This gave an aqueous dispersion with a viscosity of 900 mPas. A 0.5% strength aqueous solution of this dispersion had a viscosity of 34 000 mPas at pH 7.

### Example 35

Example 27 was repeated with the sole exception that instead of pentaerythritol triallyl ether, now 4.35 g of a 10% strength aqueous solution of triallylamine were used as crosslinker. This gave an aqueous dispersion with a viscosity of 1000 mPas. A 0.5% strength aqueous solution of this dispersion had a viscosity of 38 000 mPas at pH 7.

### Example 36

Example 22 was repeated with the exceptions that, as the initial charge, a solution of 87.5 g of a block copolymer of ethylene oxide (EO) and propylene oxide (PO) with a content of EO of 20% and a molar mass of 1750 g/mol of the polypropylene glycol block, 87.5 g of a copolymer of 20.3 parts by weight of propylene oxide and 14.2 parts by weight of ethylene oxide, 250 g of a 10% strength aqueous solution of a copolymer of 60 mol % of methacrylic acid, 20 mol % of the Na salt of acrylamidomethylpropanesulfonic acid and 20 mol % of a vinylimidazole quaternized with methyl chloride and 359 g of completely demineralized water was used. This gave a dispersion with a viscosity of 2000 m Pas. A 0.5% strength aqueous solution of this dispersion had a viscosity of 20 000 mPas at pH 7.

### Example 37

Example 22 was repeated with the exceptions that, as the initial charge, a solution 87.5 g of a block copolymer of ethylene oxide (EO) and propylene oxide (PO) with a content of EO of 20% and a molar mass of 1750 g/mol of the polypropylene glycol block, 87.5 g of a copolymer of 20.3 parts by weight of propylene oxide and 14.2 parts by weight of ethylene oxide, 250 g of a 10% strength aqueous solution of a copolymer of 20 mol % of methyl acrylate, 69 mol % of the Na salt of acrylamidomethyl-propanesulfonic acid, 10 mol % of a vinylimidazole quaternized with methyl chloride and 1 mol % of styrene and 359 g of completely demineralized water was used. This gave a dispersion with a viscosity of 900 mPas. A 0.5% strength aqueous solution of this dispersion had a viscosity of 22 000 mPas at pH 7.

### Example 38

Example 22 was repeated with the exceptions that, as the initial charge, a solution of 175 g of polyethylene glycol of molar mass M_{w} 1500, 250 g of a 10% strength aqueous solution of a copolymer of 80 mol % of methacrylic acid and 20 mol % of the Na salt of acrylamidomethylpropanesulfonic acid and 359 g of completely demineralized water was used. This gave a dispersion with a viscosity of 2500 mPas. A 0.5% strength aqueous solution of this dispersion had a viscosity of 34 000 mPas at pH 7.

### Example 39

In a glass reactor which was equipped with an anchor stirrer and a device for working under nitrogen, while passing nitrogen through and with constant stirring at a speed of 200 rpm, 479.8 g of completely demineralized water, 106.7 g of a block copolymer of ethylene oxide (EO) and propylene oxide (PO) with a content of EO of 20% and a molar mass of 1750 g/mol of the polypropylene glycol block, 53.3 g of a copolymer of 20.3 parts by weight of propylene oxide and 14.2 parts by weight of ethylene oxide and 110 g of a 20% strength aqueous solution of a copolymer of 59 parts by weight of acrylamidomethylpropanesulfonic acid, 20 parts by weight of methyl acrylate, 20 parts by weight of acrylic acid and 1 part by weight of styrene were initially introduced.

Then, with stirring (200 rpm), a mixture of 139.2 g of acrylic acid and 69.55 g of a 50% strength aqueous solution of a dimethylaminoethyl methacrylate quaternized with diethyl sulfate and 1.5 g of pentaerythritol triallyl ether (70% strength) was added dropwise over the course of 5 minutes and the resulting emulsion was heated to 40° C. After adding a solution of 0.2 g of Azostarter VA-044 in 10 g of water and rinsing the metering device with 10 g of water, the reaction mixture was heated to a temperature of 40° C. and held at this temperature for 4 hours. Then, a solution of 0.3 g of Azostarter VA-044 in 10 g of completely demineralized water was metered in, the metering device was rinsed with 10 g of completely demineralized water and the reaction mixture was then stirred for a further 1 hour for the afterpolymerization at 40° C. This gave a milky white dispersion with a viscosity of 1850 mPas. A 0.5% strength aqueous solution had a viscosity of 12 150 mPas at pH 7.

### Example 40

In a 2 l glass reactor which was equipped with an anchor stirrer and a device for working under nitrogen, while passing nitrogen through, 600.59 g of distilled water, 160 g of a block copolymer of ethylene oxide (EO) and propylene oxide (PO) with a content of EO of 40% and a molar mass of 1750 g/mol of the polypropylene glycol block (Pluronic®-PE 6400) and 89.41 g of stabilizer 16 were initially introduced. Then, with stirring (200 rpm) at room temperature, 150 g of acrylic acid were added dropwise over the course of 10 minutes. After adding 0.2 g of Azostarter V-65, the reaction mixture was heated to an internal temperature of 40° C. and held at this temperature. After 1 hour, a further 0.3 g of Azostarter V-65 was added and after 5 hours 0.4 g of Azostarter VA-044 was added. This gave a milky white dispersion with a viscosity of 650 mPas (spindle 4, 20 rpm).

An aqueous solution, 2% strength by weight based on polyacrylic acid, had, following pH adjustment to 7 with triethanolamine, a viscosity of 550 mPas (spindle 4, 20 rpm).

### Example 41

In a 2 l glass reactor which was equipped with an anchor stirrer and a device for working under nitrogen, while passing nitrogen through, 575.55 g of distilled water, 160 g of a block copolymer of ethylene oxide (EO) and propylene oxide (PO) with a content of EO of 40% and a molar mass of 1750 g/mol of the polypropylene glycol block (Pluronic® PE 6400) and 89.41 g of stabilizer 16 were initially introduced. Then, with stirring (200 rpm) at room temperature, 175 g of acrylic acid were added dropwise over the course of 10 minutes. After adding 0.3 g of Azostarter VA-044, the reaction mixture was heated to an internal temperature of 40° C. and held at this temperature until the end of the polymerization. This gave a milky white dispersion with a viscosity of 1550 mPas (spindle 4, 20 rpm). The dispersed particles had a particle size of 5 to 10 μm with individual larger particles up to 50 μm.

An aqueous solution, 2% strength by weight based on polyacrylic acid, had, following pH adjustment to 7 with triethanolamine, a viscosity of 600 mPas (spindle 4, 20 rpm).

### Example 42

In a 2 l glass reactor which was equipped with an anchor stirrer and a device for working under nitrogen, while passing nitrogen through, 560.59 g of distilled water, 175 g of a block copolymer of ethylene oxide (EO) and propylene oxide (PO) with a content of EO of 40% and a molar mass of 1750 g/mol of the polypropylene glycol block (Pluronic®PE 6400) and 89.41 g of stabilizer 16 were initially introduced. Then, with stirring (200 rpm) at room temperature, 175 g of acrylic acid and 0.875 g of triallylamine were added dropwise over the course of 10 minutes. After adding 0.3 g of Azostarter VA-044, the reaction mixture was heated to an internal temperature of 40° C. and held at this temperature until the end of the polymerization. This gave a milky white dispersion with a viscosity of 4000 mPas (spindle 4, 20 rpm). The dispersed polymer had a particle size of 5 to 10 μm with individual larger particles up to 40 μm.

An aqueous solution,1% strength by weight based on polyacrylic acid, had, following pH adjustment to 7 with triethanolamine, a viscosity of 11 600 mPas (spindle 6 20 rpm),

### Example 43

In a 2 l glass reactor which was equipped with an anchor stirrer and a device for working under nitrogen, while passing nitrogen through, 560.59 g of distilled water, 175 g of a block copolymer of EO and PO with a content of EO of 30% and a molar mass of 1100 g/mol of the polypropylene glycol block (Pluronic®PE 4300) and 89.41 g of stabilizer 16 were initially introduced. Then, with stirring (200 rpm) at room temperature, 175 g of acrylic acid and 0.875 g of triallylamine were added dropwise over the course of 10 minutes. After adding 0.3 g of Azostarter VA-044, the mixture was heated to an internal temperature of 40° C. and held at this temperature until the end of the polymerization. This gave a milky white dispersion with a viscosity of 6700 mPas (spindle 5, 20 rpm).

An aqueous solution, 1 % strength by weight based on polyacrylic acid, had, following pH adjustment to 7 with triethanolamine, a viscosity of 11 500 mPas (spindle 6 20 rpm).

### Example 44

In a 2 l glass reactor which was equipped with an anchor stirrer and a device for working under nitrogen, while passing nitrogen through, 560.59 g of distilled water, 175 g of a block copolymer of EO and PO with a content of EO of 30% and a molar mass of 1100 g/mol of the polypropylene glycol block (Pluronic®PE 4300) and 89.41 g of stabilizer 16 were initially introduced. Then, with stirring (200 rpm) at room temperature, 173.55 g of acrylic acid and 1.75 g of triallylamine were added dropwise over the course of 10 minutes. After adding 0.3 g of Azostarter VA-044, the mixture was heated to an internal tempreature of 40° C. and kept at this temperature until the end of the polymerization. This gave a milky white dispersion with a viscosity of 16 000 mPas (spindle 4, 20 rpm). The dispersed polymer particles had a particle size of from 5 to 10 μm.

An aqueous solution, 1 % strength by weight based on polyacrylic acid, had, following pH adjustment to 7 with triethanolamine, a viscosity of 21 000 mPas (spindle 6, 20 rpm).

### Example 45

In a 2 l glass reactor which was equipped with an anchor stirrer and a device for working under nitrogen, while passing nitrogen through, 515.88 g of distilled water, 175 g of a block copolymer of EO and PO with a content of EO of 30% and a molar mass of 1100 g/mol of the polypropylene glycol block (Pluronic®PE 4300) and 134.12 g of stabilizer 16 were initially introduced. Then, with stirring (200 rpm) at room temperature, 173.55 g of acrylic acid and 1.75 g of triallylamine were added dropwise over the course of 10 minutes. After adding 0.3 g of Azostarter VA-044, the reaction mixture was heated to an internal temperature of 40° C. and held at this temperature until the end of the polymerization. This gave a milky white thixotropic dispersion. The dispersion has a particle size of from 8 to 20 μm.

An aqueous solution, 1 % strength by weight based on polyacrylic acid, had, following pH adjustment to 7 with triethanolamine, a viscosity of 34 000 mPas (spindle 6, 20 rpm).

### Example 46

In a 2 l glass reactor which was equipped with an anchor stirrer and a device for working under nitrogen, while passing nitrogen through, 552.38 g of distilled water, 175 g of a block copolymer of EO and PO with a content of EO of 30% and a molar mass of 1100 g/mol of the polypropylene glycol block (Pluronic®PE 4300) and 97.32 g of stabilizer 17 were initially introduced. Then, with stirring (200 rpm) at room temperature, 173.55 g of acrylic acid and 1.75 g of triallylamine were added dropwise over the course of 10 minutes. After adding 0.3 g of Azostarter VA-044, the mixture was heated to an internal temperature of 40° C. and held at this temperature until the end of the polymerization. This gave a milky white dispersion with a viscosity of 42 000 mPas (spindle 4, 20 rpm). The dispersed polymer particles had a particle size of from 5 to 10 μm.

An aqueous solution, 1 % strength by weight based on polyacrylic acid, had, following pH adjustment to 7 with triethanolamine, a viscosity of 13 000 mPas (spindle 6, 20 rpm).

### Example 47

In a 2 l glass reactor which was equipped with an anchor stirrer and a device for working under nitrogen, while passing nitrogen through, 560.59 g of distilled water, 175 g of a block copolymer of EO and PO with a content of EO of 30% and a molar mass of 1100 g/mol of the polypropylene glycol block (Pluronic®PE 4300) and 89.41 g of stabilizer 16 were initially introduced. Then, with stirring (200 rpm) at room temperature, 173.25 g of acrylic acid and 1.75 g of N,N′-divinylethyleneurea were added dropwise over the course of 10 minutes. After adding 0.3 g of Azostarter VA-044, the mixture was heated to an internal temperature of 40° C. and held at this temperature until the end of the polymerization. This gave a milky white dispersion with a viscosity of 4950 mPas (spindle 4, 20 rpm). The dispersion had a particle size of from 5 to 10 μm.

An aqueous solution, 1 % strength by weight based on polyacrylic acid, had, following pH adjustment to 7 with triethanolamine, a viscosity of 3000 mPas (spindle 6, 100 rpm).

### Example 48

In a 2 l glass reactor which was equipped with an anchor stirrer and a device for working under nitrogen, while passing nitrogen through, 556.5 g of distilled water, 175 g of a block copolymer of EO and PO with a content of EO of 30% and a molar mass of 1100 g/mol of the polypropylene glycol block (Pluronic®PE 4300) and 93.2 g of stabilizer 18 were initially introduced. Then, with stirring (200 rpm) at room temperature, 172.5 g of acrylic acid and 1.25 g of pentaerythritol triallyl ether (70% strength) were added dropwise over the course of 10 minutes. After adding 0.3 g of Azostarter VA-044, the reaction mixture was heated to an internal temperature of 40° C. and held at this temperature until the end of the polymerization. This gave a milky white dispersion with a viscosity of 13 000 mPas (spindle 5, 20 rpm, 30° C.). The dispersed polymer particles of the dispersion had a particle size of from 15 to 35 μm.

An aqueous solution, 0.25% strength by weight based on polyacrylic acid, had, following pH adjustment to 7 with triethanolamine, a viscosity of 12000 mPas (spindle 7, 10 rpm).

### Example 49

In a 2 l glass reactor which was equipped with an anchor stirrer and a device for working under nitrogen, while passing nitrogen through, 556.5 g of distilled water, 175 g of a block copolymer of EO and PO with a content of EO of 30% and a molar mass of 1100 g/mol of the polypropylene glycol block (Pluronic®PE 4300) and 93.2 g of stabilizer 18 were initially introduced. Then, with stirring (200 rpm) at room temperature, 174 g of acrylic acid and 1.0 g of pentaerythritol triallyl ether (70% strength) were added dropwise over the course 10 minutes. After adding 0.2 g of Azostarter VA-044, the reaction mixture was heated to an internal temperature of 40° C. and held at this temperature until the end of the polymerization. At the end of the polymerization, 0.4 g of Azostarter VA-044 were added for the afterpolymerization. This gave a milky white dispersion with a viscosity of 68 000 mPas (spindle 4, 2.5 rpm). The dispersion had a particle size of from 6 to 30 μm.

An aqueous solution, 0.5% strength by weight based on polyacrylic acid, had, following pH adjustment to 7 with triethanolamine, a viscosity of 33 000 mPas (spindle 7, 20 rpm).

### Example 50

In a 2 l glass reactor which was equipped with an anchor stirrer and a device for working under nitrogen, while passing nitrogen through, 560.39 g of distilled water, 175 g of a block copolymer of EO and PO with a content of EO of 30% and a molar mass of 1100 g/mol of the polypropylene glycol block (Pluronic®PE 4300) and 89.41 g of stabilizer 16 were initially introduced. Then, with stirring (200 rpm) at room temperature, 174 g of acrylic acid and 1.0 g of pentaerythritol triallyl ether (70% strength) were added dropwise over the course of 10 minutes. After adding 0.2 g of Azostarter VA-044, the reaction mixture was heated to an internal temperature of 40° C. and held at this temperature until the end of the polymerization. At the end of the actual polymerization, 0.4 g of Azostarter VA-044 was added for the afterpolymerization. This gave a milky white dispersion with a viscosity of 15 400 mPas (spindle 4, 10 rpm). The dispersion had a particle size of from 6 to 30 μm.

An aqueous solution, 0.5% strength by weight based on polyacrylic acid, had, following pH adjustment to 7 with triethanolamine, a viscosity of 30 000 mPas (spindle 7, 20 rpm).

### Application Examples of Cosmetic Preparations

The quantitative data below are in % by weight, unless expressly noted otherwise. The amounts of the copolymers used according to the invention are given in % by weight of polymer as solid. If the polymer is used in the form of a dispersion, the stated required amount of polymer must be used in the form of the corresponding amount of dispersion. The % parts by weight of polymer arise from the data of the preparation examples. This applies analogously if the polymer is used in the form of a solution. Instead of or in addition to the paraffin oil used in the following examples, isoalkane mixtures, as described, for example, in the patent application DE 10 2004 018 753, are also advantageously used.

It is particularly preferred to use an isoalkane mixture whose ¹H-NMR spectrum in the region of a chemical shift 6 from 0.6 to 1.0 ppm, based on tetramethylsilane, has an area integral from 25 to 70%, based on the total integral area. Such isoalkane mixtures and methods for their production are described in the unpublished patent application DE 10 2005 022 021.5.

The abovementioned isoalkane mixtures can advantageously also be used as mixtures with isohexadecane or as mixtures with Paraffinum liquidum in each case in the weight ratio from 10:1 to 1:10 instead of pure paraffin oil.

If, in the following application examples, the polymers of Examples 2 to 50 are mentioned, then this is understood as meaning the polymers of the above Preparation Examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, and any suitable mixture thereof.

### Application Example 1

### Setting Compositions for Hair Gels

| | INCI |
|---|---|
| 0.50% Carbopol ® 940 | Carbomer |
| 3.00% polymer from Example 1 | |
| 0.10% phytantriol | |
| 0.50% panthenol | |
| q.s. perfume oil | |
| q.s preservative | |
| ad 100% water | |

The example can be repeated with the polymers of the other Examples 1 to 50. In each case, a refreshing gel with good properties is obtained.

### Application Example 2

### Hair Shampoo or Shower Gel

| | INCI |
|---|---|
| 0.50% polymer from Example 18 | |
| 40.00% Texapon ® NSO | Sodium Laureth Sulfate |
| 5.00% Tego Betain ® L 7 | Cocamidopropyl Betaine |
| 5.00% Plantacare ® 2000 | Decyl Glucoside |
| 1.00% propylene glycol | |
| q.s. citric acid | |
| q.s. preservative | |
| 1.00% sodium chloride | |
| ad 100% water | |

The example can be repeated with the polymers of the other Examples 1 to 50. In each case, a hair shampoo or shower gel with good properties is obtained.

### Application Example 3

### Skin Cream

Water/oil cream emulsions were prepared according to the following formulation:

| Additive | | % by wt. |
|---|---|---|
| Cremophor ® A 6 | ceteareth-6 and stearyl alcohol | 2.0 |
| Chremophor ® A 25 | ceteareth-25 | 2.0 |
| Lanette O | cetearyl alcohol | 2.0 |
| Imwitor ® 960 K | glyceryl stearate SE | 3.0 |
| Paraffin oil | | 5.0 |
| Jojoba oil | | 4.0 |
| Luvitol ® EHO | cetearyl octanoate | 3.0 |
| ABIL ® 350 | dimethicone | 1.0 |
| Amerchol ® L 101 | mineral oil and lanolin alcohol | 3.0 |
| Veegum ® Ultra | magnesium aluminum silicate | 0.5 |
| 1,2-Propylene glycol | propylene glycol | 5.0 |
| Abiol ® | imidazolindinylurea | 0.3 |
| Phenoxyethanol | | 0.5 |
| D-panthenol USP | | 1.0 |
| Polymer from Example 1 | | 0.5 |
| Water | | ad 100 |

The example can be repeated with the polymers from Examples 1 to 50. In each case, a skin cream with good properties is obtained.

### Aplication Example 4

### Shower Gels

Shower gel formulations according to the following formulation were prepared:

| Additive | | % by wt. |
|---|---|---|
| Texapon ® NSO | sodium laureth sulfate | 40.0 |
| Tego Betain ® L7 | cocamidopropylbetaine | 5.0 |
| Plantacare ® 2000 | decyl glucoside | 5.0 |
| Perfume | | 0.2 |
| Polymer from Example 17 | | 0.2 |
| Euxyl ® K 100 | benzyl alcohol, | 0.1 |
| | methylchloroisothiazolinone, | |
| | methylisothiazolinone | |
| D-panthenol USP | | 0.5 |
| Citric acid (pH 6-7) | | q.s. |
| NaCl | | 2.0 |
| Water | | ad 100 |

The example can be repeated with the polymers of the other Examples 1 to 50. In each case, a shower gel with good properties is obtained.

### Application Example 5

### Thickener for Hair Gels

0.50% polymer from Example 1 neutralized with triethanolamine to pH 7.5

3.00% Luviskol®K 90 P

0.50% panthenol

q.s. perfume oil

q.s. preservative

ad 100% water

The example can be repeated with the polymers of the other Examples 1 to 50. In each case, a freshening gel with good properties is obtained.

### Application Example 6

### Humectant Formulations

Formulation A

| | Additive | | % by wt. |
|---|---|---|---|
| a) | Cremophor ® A6 | ceteareth-6 and stearyl | 2.0 |
| | | alcohol | |
| | Cremophor ® A25 | ceteareth-25 | 2.0 |
| | Paraffin oil (high viscosity) | | 10 |
| | Lannette ® O | cetearyl alcohol | 2.0 |
| | Stearic acid | | 3.0 |
| | Nip-Nip | methyl paraben/propyl | 0.5 |
| | | paraben 70:30 | |
| | Abiol ® | imidazoldinylurea | 0.5 |
| b) | polymer from Example 18 | | 3.0 |
| | water | | ad 100.0 |

Both phases are heated to 80° C., phase a) was stirred into phase b), homogenized and stirred until cold and then adjusted to pH 6 with 10% strength aqueous NaOH solution.

The example can be repeated with the polymers of the other Examples 1 to 50. In each case, a humectant formulation with good properties is obtained.

### Application Example 7

### O/W Creams for Skin Moisturization

| Additive | % by wt. |
|---|---|
| Glycerol monostearate | 2.0 |
| Cetyl alcohol | 3.0 |
| Paraffin oil, subliquidum | 15.0 |
| Vaseline | 3.0 |
| Caprylic/capric triglyceride | 4.0 |
| Octyldodecanol | 2.0 |
| Hydrogenated coconut fat | 2.0 |
| Cetyl phosphate | 0.4 |
| Polymer from Example 17 | 3.0 |
| Glycerol | 3.0 |
| Sodium hydroxide | q.s. |
| Perfume oil | q.s. |
| Preservative | q.s. |
| Water | ad 100 |

The example can be repeated with the polymers of the other Examples 1 to 50. In each case, an O/W skin moisturizing cream with good properties is obtained.

### Application Example 8

### O/W Lotions

| Additive | % by wt. |
|---|---|
| Stearic acid | 1.5 |
| Sorbitan monostearate | 1.0 |
| Sorbitan monooleate | 1.0 |
| Paraffin oil, subliquidum | 7.0 |
| Cetyl alcohol | 1.0 |
| Polydimethylsiloxane | 1.5 |
| Glycerol | 3.0 |
| Polymer from Example 1 | 0.5 |
| Perfume oil | q.s. |
| Preservative | q.s. |
| Water | ad 100 |

The example can be repeated with the polymers of the other Examples 1 to 50. In each case, an O/W lotion with good properties is obtained.

### Application Example 9

### W/O Creams

| Additive | % by wt. |
|---|---|
| PEG-7 hydrogenated castor oil | 4.0 |
| Wool wax alcohol | 1.5 |
| Beeswax | 3.0 |
| Triglyceride, liquid | 5.0 |
| Vaseline | 9.0 |
| Ozokerite | 4.0 |
| Paraffin oil, subliquidum | 4.0 |
| Glycerol | 2.0 |
| Polymer from Example 18 | 2.0 |
| Magnesium sulfate*7H2O | 0.7 |
| Perfume oil | q.s. |
| Preservative | q.s. |
| Water | ad 100 |

The example can be repeated with the polymers of the other Examples 1 to 50. In each case, a W/O cream with good properties is obtained.

### Application Example 10

### Hydrogels for Skin Care

| Additive | % by wt. |
|---|---|
| Polymer from Example 17 | 3.0 |
| Sorbitol | 2.0 |
| Glycerol | 3.0 |
| Polyethylene glycol 400 | 5.0 |
| Ethanol | 1.0 |
| Perfume oil | q.s. |
| Preservative | q.s. |
| Water | ad 100 |

The example can be repeated with the polymers of the other Examples 1 to 50. In each case, a hydrogel for skin care with good properties is obtained.

### Application Example 11

### Hydrodispersion Gels

| Additive | % by wt. |
|---|---|
| Polymer from Example 1 | 3.0 |
| Sorbitol | 2.0 |
| Glycerol | 3.0 |
| Polyethylene glycol 400 | 5.0 |
| Triglyceride, liquid | 2.0 |
| Ethanol | 1.0 |
| Perfume oil | q.s. |
| Preservative | q.s. |
| Water | ad 100 |

The example can be repeated with the polymers of the other Examples 1 to 50. In each case, a hydrodispersion gel with good properties is obtained.

### Application Example 12

### Liquid Soaps

| Additive | % by wt. |
|---|---|
| Coconut fatty acid, potassium salt | 15 |
| Potassium oleate | 3 |
| Glycerol | 5 |
| Polymer from Example 18 | 2 |
| Glycerol stearate | 1 |
| Ethylene glycol distearate | 2 |
| Specific additives, complexing agents, fragrances | q.s. |
| Water | ad 100 |

The example can be repeated with the polymers of the other Examples 1 to 50. In each case, a liquid soap with good properties is obtained.

### Application Example 13

### Sunscreen Emulsions Containing TiO₂ and ZnO₂

Phase A
6.00 PEG-7 Hydrogenated Castor Oil
2.00 PEG-45/Dodecyl Glycol Copolymer
3.00 Isopropyl Myristate
8.00 Jojoba (Buxus Chinensis) Oil
4.00 Octyl Methoxycinnamate (Uvinul®MC 80)
2.00 4-Methylbenzylidene Camphor (Uvinul®MBC 95)
3.00 Titanium Dioxide, dimethicone
1.00 Dimethicone
5.00 Zinc Oxide, dimethicone

Phase B
2.00 Polymer from Example 17
0.20 Disodium EDTA
5.00 Glycerol
q.s. Preservative
58.80 Dist. Water

Phase C
q.s. Perfume Oil

Preparation:

Heat phases A and B separately to about 85° C. Stir phase B into phase A and homogenize. Cool to about 40° C., add phase C and briefly homogenize again.

The example can be repeated with the polymers of the other Examples 1 to 50. In each case, a sunscreen emulsion with good properties is obtained.

### Application Example 14

### Face Cleansing Milk of the O/W Type

Phase A
1.50 Ceteareth-6
1.50 Ceteareth-25
2.00 Glyceryl Stearate
2.00 Cetyl Alcohol
10.00 Mineral Oil

Phase B
5.00 Propylene Glycol
q.s. Preservative
1.0 Polymer from Example 1
66.30 Dist. Water

Phase C
0.20 Carbomer
10.00 Cetearyl Octanoate

Phase D
0.40 Tetrahydroxypropyl Ethylenediamine

Phase E
q.s. Perfume Oil
0.10 Bisabolol

Preparation:

Heat phases A and B separately to about 80° C. Stir phase B into phase A with homogenization, briefly after-homogenize. Prepare a slurry from phase C, stir into phase AB, neutralize with phase D and after-homogenize. Cool to about 40° C., add Phase E, homogenize again.

The example can be repeated with the polymers of the other Examples 1 to 50. In each case, a face cleansing milk with good properties is obtained.

### Application Example 15

### Bodycare Creams

| Additive | % by wt. |
|---|---|
| Cremophor ® A6 Ceteareth-6 and Stearyl Alcohol | 2.0% |
| Cremophor ® A 25 Ceteareth-25 | 2.0% |
| Grape (Vitis Vinifera) Seed oil | 6.0% |
| Glyceryl stearate SE | 3.0% |
| Cetearyl alcohol | 2.0% |
| Dimethicone | 0.5% |
| Luvitol ® EHO Cetearyl Octanoate | 8.0% |
| Oxynex ® 2004 Propylene Glycol, BHT, Ascorbyl | 0.1% |
| Palmitate, Glyceryl Stearate, Citric Acid | |
| Preservative | q.s. |
| 1,2-Propylene Glycol USP | 3.0% |
| Glycerol | 2.0% |
| EDTA BD | 0.1% |
| D-Panthenol USP | 1.0% |
| Water | ad 100 |
| Polymer from Example 18 | 1.5% |
| Tocopheryl acetate | 0.% |

The example can be repeated with the polymers of the other Examples 1 to 50. In each case, a bodycare cream with good properties is obtained.

In the Application Examples below, all of the quantitve data is in % by wt.

### Application Example 16

### Liquid Makeups

Phase A
1.70 Glyceryl Stearate
1.70 Cetyl Alcohol
1.70 Ceteareth-6
1.70 Ceteareth-25
5.20 Caprylic/Capric Triglyceride
5.20 Mineral Oil

Phase B
q.s. Preservative
4.30 Propylene Glycol
2.50 Polymerfrom Example 17
59.50 Dist. Water

Phase C
q.s. Perfume Oil

Phase D
2.00 Iron Oxides
12.00 Titanium Dioxide

Preparation:

Heat phase A and phase B separately from one another to 80° C. Then mix phase B into phase A using a stirrer. Allow everything to cool 40° C. and add phase C and phase D. Homogenize again.

The example can be repeated with the polymers of the other Examples 1 to 50. In each case, a liquid makeup with good properties is obtained.

### Application Example 17

### Oil-Free Makeups

Phase A
0.35 Veegum
5.00 Butylene Glycol
0.15 Xanthan Gum

Phase B
53.0 Dist. Water
q.s. Preservative
0.2 Polysorbate®-20
1.6 Tetrahydroxypropyl Ethylenediamine

Phase C
1.0 Silica
2.0 Nylon-12
4.15 Mica
6.0 Titanium Dioxide
1.85 Iron Oxides

Phase D
4.0 Stearic Acid
1.5 Glyceryl Stearate
7.0 Benzyl Laurate
5.0 Isoeicosane
q.s. Preservative

Phase E
1.0 Dist. Water
0.5 Panthenol
0.1 Imidazolidinyl Urea
5.0 Polymerfrom Example 1

Preparation:

Wet phase A with butylene glycol, add to phase B and mix well. Heat phase AB to 75° C. Pulverize phase C feed materials, add to phase AB and homogenize well. Mix feed materials of phase D, heat to 80° C. and add to phase ABC. Mix for some time until everything is homogeneous. Transfer everything to a vessel with a propeller mixer. Mix feed materials of phase E, add to phase ABCD and mix well.

The example can be repeated with the polymers of the other Examples 1 to 50. In each case, an oil-free makeup with good properties is obtained.

### Application Example 18

### Eyeliners

Phase A
40.6 Dist. water
0.2 Disodium EDTA
q.s. Preservative

Phase B
0.6 Xanthan Gum
0.4 Veegum
3.0 Butylene Glycol
0.2 Polysorbate-20

Phase C
15.0 Iron oxide/Al Powder/Silica (e.g. Sicopeari Fantastico Gold from BASF)

Phase D
10.0 Dist. Water
30.0 Polymer from Example 18

Preparation:

Premix phase B. Using a propeller mixer, mix phase B into phase A, allowing the thickener to swell. Wet phase C with phase D, add everything to phases AB and mix well.

The example can be repeated with the polymers of the other Examples 1 to 50. In each case, an eyeliner with good properties is obtained.

### Application Example 19

### Shimmering Gels

Phase A
32.6 Dist. Water
0.1 Disodium EDTA
25.0 Carbomer (2% strength aqueous solution)
0.3 Preservative

Phase B
0.5 Dist. Water
0.5 Triethanolamine

Phase C
10.0 Dist. Water
9.0 Polymer from Example 17
1.0 Polyquaternium-46
5.0 Iron Oxide

Phase D
15.0 Dist. Water
1.0 D-Panthenol 50 P (Panthenol and Propylene Glycol)

Preparation:

Using a propeller mixer, thoroughly mix the feed materials of phase A in the given order. Then add phase B to phase A. Stir slowly until everything is homogeneous. Thoroughly homogenize phase C until the pigments are well distributed. Add phase C and phase D to phase AB and mix well.

The example can be repeated with the polymers of the other Examples 1 to 50. In each case, a shimmering gel with good properties is obtained.

### Application Example 20

### Water-Resistant Mascaras

Phase A
46.7 Dist. Water
3.0 Lutrol®E 400 (PEG-8)
0.5 Xanthan Gum
q.s. Preservative
0.1 Imidazolidinylurea
1.3 Tetrahydroxypropylethylenediamine

Phase B
8.0 Carnauba Wax
4.0 Beeswax
4.0 Isoeicosane
4.0 Polyisobutene
5.0 Stearic Acid
1.0 Glyceryl Stearate
q.s. Preservative
2.0 Benzyl Laurate

Phase C
10.0 Iron oxide/Al Powder/Silica (z.B. Sicopearl Fantastico Gold® from BASF)

Phase E
8.0 Polyurethane-1
2.0 Polymer from Example 1

Preparation:

Heat phase A and phase B separately from one another to 85° C. Maintain the temperature and add phase C to phase A and homogenize until the pigments are uniformly distributed. Add phase B to phases AC and homogenize for 2-3 minutes. Then add phase E and stir slowly. Allow everything to cool to room temperature.

The example can be repeated with the polymers of the other Examples 1 to 50. In each case, a water-resistant mascara with good properties is obtained.

### Application Example 21

### Sunscreen Gels

Phase A
1.00 PEG-40 Hydrogenated Castor Oil
8.00 Octyl Methoxycinnamate (Uvinul MC 80® from BASF)
5.00 Octocrylene (Uvinul N 539® from BASF)
0.80 Octyl Triazone (Uvinul T 150® from BASF)
2.00 Butyl Methoxydibenzoylmethane (Uvinul BMBM® from BASF)
2,00 Tocopheryl Acetate
q.s. Perfume Oil

Phase B
2.50 Polymer from Example 18
0.30 Acrylates/C10-30 Alkyl Acrylate Crosspolymer
0.20 Carbomer
5.00 Glycerol
0.20 Disodium EDTA
q.s. Preservative
72.80 Dist. Water

Phase C
0.20 Sodium Hydroxide

Preparation:

Mix the components of phase A. Allow phase B to swell and stir into phase A with homogenization. Neutralize with phase C and homogenize again.

The example can be repeated with the polymers of the other Examples 1 to 50. In each case, a sunscreen gel with good properties is obtained.

### Application Example 22

### Sunscreen Lotions

Phase A
6.00 Octyl Methoxycinnamate (Uvinul®MC 80 from BASF)
2.50 4-Methylbenzylidene Camphor (Uvinul®MBC 95 from BASF)
1.00 Octyl Triazone (Uvinul®T 150 TM from BASF)
2.00 Butyl Methoxydibenzoylmethane (Uvinul®BMBM from BASF)
2.00 PVP/Hexadecene Copolymer
5.00 PPG-3 Myristyl Ether
0.50 Dimethicone
0.10 BHT, Ascorbyl Palmitate, Citric Acid, Glyceryl Stearate, Propylene Glycol
2.00 Cetyl Alcohol
2.00 Potassium Cetyl Phosphate

Phase B
2.50 Polymer from Example 17
5.00 Propylene Glycol
0.20 Disodium EDTA
q.s. Preservative
63.92 Dist. Water

Phase C
5.00 Mineral Oil
0.20 Carbomer

Phase D
0.08 Sodium Hydroxide

Phase E
q.s. Perfume Oil

Preparation:

Heat phases A and B separately to about 80° C. Stir phase B into phase A with homogenization, briefly after-homogenize. Prepare a slurry from phase C, stir into phase AB, neutralize with phase D and after-homogenize. Cool to about 40° C., add phase E, homogenize again.

The example can be repeated with the polymers of the other Examples 1 to 50. In each case, a sunscreen lotion with good properties is obtained.

### Application Example 23

### Peelable Face Masks

Phase A
57.10 Dist. Water
6.00 Polyvinyl Alcohol
5.00 Propylene Glycol

Phase B
20.00 Alcohol
4.00 PEG-32
q.s Perfume Oil

Phase C
5.00 Polyquaternium-44
2.70 Polymer from Example 1
0.20 Allantoin

Preparation:

Heat phase A to at least 90° C. and stir until dissolved. Dissolve phase B at 50° C. and stir into phase A. At about 35° C., compensate for the loss of ethanol. Add phase C and stir in.

The example can be repeated with the polymers of the other Examples 1 to 50. In each case, a peelable face mask with good properties is obtained.

### Application Example 24

### Face Masks

Phase A
3.00 Ceteareth-6
1.50 Ceteareth-25
5.00 Cetearyl Alcohol
6.00 Cetearyl Octanoate
6.00 Mineral Oil
0.20 Bisabolol
3.00 Glyceryl Stearate

Phase B
2.00 Propylene Glycol
5.00 Panthenol
2.80 Polymer from Example 18
q.s. Preservative
65.00 Dist. Water

Phase C
q.s. Perfume oil
0.50 Tocopheryl Acetate

Preparation:

Heat phases A and B separately to about 80° C. Stir phase B into phase A with homogenization, briefly after-homogenize. Cool to about 40° C., add phase C, homogenize again.

The example can be repeated with the polymers of the other Examples 1 to 50. In each case, a face mask with good properties is obtained.

### Application Example 25

### Body Lotion Foams

Phase A
1.50 Ceteareth-25
1.50 Ceteareth-6
4.00 Cetearyl Alcohol
10.00 Cetearyl Octanoate
1.00 Dimethicone

Phase B
3.00 Polymer from Example 17
2.00 Panthenol
2.50 Propylene Glycol
q.s. Preservative
74.50 Dist. Water

Phase C
q.s. Perfume oil

Preparation:

Heat phases A and B separately to about 80° C. Stir phase B into phase A and homogenize. Cool to about 40° C., add phase C and briefly homogenize again. Bottling: 90% active ingredient and 10% propane/butane at 3.5 bar (20° C.).

The example can be repeated with the polymers of the other Examples 1 to 50. In each case, a body lotion foam with good properties is obtained.

### Application Example 26

### Face Tonics for Dry and Sensitive Skin

Phase A
2.50 PEG-40 Hydrogenated Castor Oil
q.s. Perfume Oil
0.40 Bisabolol

Phase B
3.00 Glycerol
1.00 Hydroxyethyl Cetyldimonium Phosphate
5.00 Witch Hazel (Hamamelis Virginiana) Distillate
0.50 Panthenol
0.50 Polymer from Example 1
q.s. Preservative
87.60 Dist. Water

Preparation:

Dissolve phase A until clear. Stir phase B into phase A.

The example can be repeated with the polymers of the other Examples 1 to 50. In each case, a face tonic with good properties is obtained.

### Application Example 27

### Face Washing Pastes with Peeling Effect

Phase A
70.00 Dist. Water
3.00 Polymer from Example 18
1.50 Carbome
q.s. Preservative

Phase B
q.s. Perfume Oil
7.00 Potassium Cocoyl Hydrolyzed Protein
4.00 Cocamidopropyl Betaine

Phase C
1.50 Triethanolamine

Phase D
13.00 Polyethylene (Luwax®A from BASF)

Preparation:

Allow phase A to swell. Dissolve phase B until clear. Stir phase B into phase A. Neutralize with phase C. Then stir in phase D.

The example can be repeated with the polymers of the other Examples 1 to 50. In each case, a face washing paste with good properties is obtained.

### Application Example 28

### Face Soaps

Phase A
25.0 Potassium Cocoate
20.0 Disodium Cocoamphodiacetate
2.0 Lauramide DEA
1.0 Glycol Stearate
2.0 Polymer from Example 17
50.0 Dist. Water
q.s. Citric Acid

Phase B
q.s. Preservative
q.s. Perfume oil

Preparation:

Heat phase A to 70° C. with stirring until everything is homogeneous. pH to 7.0 to 7.5 with citric acid. Allow everything to cool to 50° C. and add phase B.

The example can be repeated with the polymers of the other Examples 1 to 50. In each case, a face soap with good properties is obtained.

### Application Example 29

### Transparent Soaps

4.20 Sodium Hydroxide
3.60 Dist. Water
2.0 Polymer from Example 1
22.60 Propylene Glycol
18.70 Glycerol
5.20 Cocoamide DEA
10.40 Cocamine Oxide
4.20 Sodium Lauryl Sulfate
7.30 Myristic Acid
16.60 Stearic Acid
5.20 Tocopherol

Preparation:

Mix all of the ingredients. Melt the mixture at 85° C. until clear. Immediately pour out into the mold.

The example can be repeated with the polymers of the other Examples 1 to 50. In each case, a transparent soap with good properties is obtained.

### Application Example 30

### Peeling Creams, O/W Type

Phase A
3.00 Ceteareth-6
1.50 Ceteareth-25
3.00 Glyceryl Stearate
5.00 Cetearyl Alcohol, Sodium Cetearyl Sulfate
6.00 Cetearyl Octanoate
6.00 Mineral Oil
0.20 Bisabolol

Phase B
2.00 Propylene Glycol
0.10 Disodium EDTA
3.00 Polymer from Example 18
q.s. Preservative
59.70 Dist. Water

Phase C
0.50 Tocopheryl Acetate
q.s. Perfume Oil

Phase D
10.00 Polyethylene

Preparation:

Heat phases A and B separately to about 80° C. Stir phase B into phase A and homogenize. Cool to about 40° C., add phase C and briefly homogenize again. Then stir in phase D.

The example can be repeated with the polymers of the other Examples 1 to 50. In each case, a peeling cream with good properties is obtained.

### Application Example 31

### Shaving Foams

6.00 Ceteareth-25
5.00 Poloxamer 407
52.00 Dist. Water
1.00 Triethanolamine
5.00 Propylene Glycol
1.00 PEG-75 Lanolin Oil
5.00 Polymer from Example 17
q.s. Preservative
q.s. Perfume oil
25.00 Sodium Laureth Sulfate

Preparation:

Weigh everything together, then stir until dissolved. Bottling: 90 parts of active substance and 10 parts of propane/butane mixture 25:75.

The example can be repeated with the polymers of the other Examples 1 to 50. In each case, a shaving foam with good properties is obtained.

### Application Example 32

### After Shave Balm

Phase A
0.25 Acrylates/C10-30 Alkyl Acrylate Crosspolymer
1.50 Tocopheryl Acetate
0.20 Bisabolol
10.00 Caprylic/Capric Triglyceride
q.s. Perfume Oil
1.00 PEG-40 Hydrogenated Castor Oil

Phase B
1.00 Panthenol
15.00 Alcohol
5.00 Glycerol
0.05 Hydroxyethyl Cellulose
1.92 Polymer from Example 1
64.00 Dist. Water

Phase C
0.08 Sodium Hydroxide

Preparation:

Mix the components of phase A. Stir phase B into phase A with homogenization, briefly after-homogenize. Neutralize with phase C and homogenize again.

The example can be repeated with the polymers of the other Examples 1 to 50. In each case, an after shave balm with good properties is obtained.

### Application Example 33

### Bodycare Creams

Phase A
2.00 Ceteareth-6
2.00 Ceteareth-25
2.00 Cetearyl Alcohol
3.00 Glyceryl Stearate SE
5.00 Mineral Oil
4.00 Jojoba (Buxus Chinensis) Oil
3.00 Cetearyl Octanoate
1.00 Dimethicone
3.00 Mineral Oil, Lanolin Alcohol

Phase B
5.00 Propylene Glycol
0.50 Veegum
1.00 Panthenol
1.70 Polymer from Example 18
6.00 Polyquaternium-44
q.s. Preservative
60.80 Dist. Water

Phase C
q.s. Perfume oil

Preparation:

Heat phases A and B separately to about 80° C. Homogenize phase B. Stir phase B into phase A with homogenization, briefly after-homogenize. Cool to about 40° C., add phase C and briefly homogenize again.

The example can be repeated with the polymers of the other Examples 1 to 50. In each case, a bodycare cream with good properties is obtained.

### Application Example 34

### Toothpastes

Phase A
34.79 Dist. Water
3.00 Polymer from Example 17
0.30 Preservative
20.00 Glycerol
0.76 Sodium Monofluorophosphate

Phase B
1.20 Sodium Carboxymethylcellulose

Phase C
0.80 Aroma Oil
0.06 Saccharin
0.10 Preservative
0.05 Bisabolol
1.00 Panthenol
0.50 Tocopheryl Acetate
2.80 Silica
1.00 Sodium Lauryl Sulfate
7.90 Dicalcium Phosphate Anhydrate
25.29 Dicalcium Phosphate Dihydrate
0.45 Titanium Dioxide

Preparation:

Dissolve phase A. Sprinkle phase B into phase A and dissolve. Add phase C and stir under reduced pressure at RT for about 45 min.

The example can be repeated with the polymers of the other Examples 1 to 50. In each case, a toothpaste with good properties is obtained.

### Application Example 35

### Mouthwash

Phase A
2.00 Aroma Oil
4.00 PEG-40 Hydrogenated Castor Oil
1.00 Bisabolol
30.00 Alcohol

Phase B
0.20 Saccharin
5.00 Glycerol
q.s. Preservative
5.00 Poloxamer 407
0.5 Polymer from Example 1
52.30 Dist. Water

Preparation:

Dissolve phase A and phase B separately until clear. Stir phase B into phase A.

The example can be repeated with the polymers of the other Examples 1 to 50. In each case, a mouthwash with good properties is obtained.

### Application Example 36

### Denture Adhesive

Phase A
0.20 Bisabolol
1.00 Beta-Carotene
q.s. Aroma Oil
20.00 Cetearyl Octanoate
5.00 Silica
33.80 Mineral Oil

Phase B
5.00 Polymer from Example 18
35.00 PVP (20% strength solution in water)

Preparation:

Thoroughly mix phase A. Stir phase B into phase A.

The example can be repeated with the polymers of the other Examples 1 to 50. In each case, a denture adhesive with good properties is obtained.

### Application Example 37

### Skincare Cream, O/W Type

Phase A
8.00 Cetearyl Alcohol
2.00 Ceteareth-6
2.00 Ceteareth-25
10.00 Mineral Oil
5.00 Cetearyl Octanoate
5.00 Dimethicone

Phase B
3.00 Polymer from Example 17
2.00 Panthenol, Propylene Glycol
q.s. Preservative
63.00 Dist. Water

Phase C
q.s. Perfume Oil

Preparation:

Heat phases A and B separately to about 80° C. Stir phase B into phase A with homogenization, briefly after-homogenize. Cool to about 40° C., add phase C, homogenize again.

The example can be repeated with the polymers of the other Examples 1 to 50. In each case, a skincare cream with good properties is obtained.

### Application Example 38

### Skincare Creams, W/O Type

Phase A
6.00 PEG-7 Hydrogenated Castor Oil
8.00 Cetearyl Octanoate
5.00 Isopropyl Myristate
15.00 Mineral Oil
2.00 PEG-45/Dodecyl Glycol Copolymer
0.50 Magnesium Stearate
0.50 Aluminum Stearate

Phase B
3.00 Glycerol
3.30 Polymer from Example 1
0.70 Magnesium Sulfate
2.00 Panthenol
q.s. Preservative
48.00 Dist. Water

Phase C
1.00 Tocopherol
5.00 Tocopheryl Acetate
q.s. Perfume Oil

Preparation:

Heat phases A and B separately to about 80° C. Stir phase B into phase A and homogenize. Cool to about 40° C., add phase C and briefly homogenize again.

The example can be repeated with the polymers of the other Examples 1 to 50. In each case, a skincare cream with good properties is obtained.

### Application Example 39

### Lipcare Creams

Phase A
10.00 Cetearyl Octanoate
5.00 Polybutene

Phase B
0.10 Carbomer

Phase C
2.00 Ceteareth-6
2.00 Ceteareth-25
2.00 Glyceryl Stearate
2.00 Cetyl Alcohol
1.00 Dimethicone
1.00 Benzophenone-3
0.20 Bisabolol
6.00 Mineral Oil

Phase D
8.00 Polymer from Example 18
3.00 Panthenol
3.00 Propylene Glycol
q.s. Preservative
54.00 Dist. Water

Phase E
0.10 Triethanolamine

Phase F
0.50 Tocopheryl Acetate
0.10 Tocopherol
q.s. Perfume Oil

Preparation:

Dissolve phase A until clear. Add phase B and homogenize. Add phase C and melt at 80° C. Heat phase D to 80° C. Add phase D to phase ABC and homogenize. Cool to about 40° C., add phase E and phase F. homogenize again.

The example can be repeated with the polymers of the other Examples 1 to 50. In each case, a lipcare cream with good properties is obtained.

### Application Example 40

### Glossy Lipsticks

Phase A
5.30 Candelilla (Euphorbia Cerifera) Wax
1.10 Bees Wax
1.10 Microcrystalline Wax
2.00 Cetyl Palmitate
3.30 Mineral Oil
2.40 Castor Oil, Glyceryl Ricinoleate, Octyldodecanol, Carnauba, Candelilla Wax,
0.40 Bisabolol
16.00 Cetearyl Octanoate
2.00 Hydrogenated Coco-Glycerides
q.s. Preservative
1.00 Polymer from Example 17
60.10 Castor (Ricinus Communis) Oil
0.50 Tocopheryl Acetate

Phase B
0.80 C.I.14 720:1, Acid Red 14 Aluminum Lake

Phase C
4.00 Mica, Titanium Dioxide

Preparation:

Weigh in the components of phase A and melt. Work in phase B until homogeneous. Add phase C and stir in. Cool to room temperature with stirring.

The example can be repeated with the polymers of the other Examples 1 to 50. In each case, a lipstick with good properties is obtained.

### Application Example 41

### Formulation for Aerosol Hair Foam:

2.00% Polymerfrom Example 1
2.00% Luviquat®Mono LS (Cocotrimonium methyl sulfate)
67.7% Water
10.0 Propane/Butane 3.5 bar (20° C.)
q.s. Perfume Oil

### Comparative Example:

2.00% Luviquat®Hold (Polyquaternium-46)
2.00% Luviquat®Mono LS (Cocotrimonium methyl sulfate)
67.7% Water
10.0 Propane/Butane 3.5 bar (20° C.)
q.s. Perfume Oil

The example can be repeated with the polymers of the other Examples 1 to 50. In each case, a lipstick with good properties is obtained.

### Application Example 42

### Aerosol Hair Foam

| | INCI |
|---|---|
| 4.00% Polymer from Example 18 | |
| 0.20% Cremophor A 25 | Ceteareth-25 |
| 1.00% Luviquat ® Mono CP | Hydroxyethyl cetyl- |
| | dimonium phosphate |
| 5.00% Ethanol | |
| 1.00% Panthenol | |
| 10.0 Propane/Butane 3.5 bar (20° C.) | |
| q.s. Perfume Oil | |
| ad 100% Water | |

The example can be repeated with the polymers of the other Examples 1 to 50. In each case, a lipstick with good properties is obtained.

### Application Example 43

### Pump Foams

| | INCI |
|---|---|
| 2.00% Polymer from Example 17 | |
| 2.00% Luviflex ® Soft (polymer content) | |
| 1.20% 2-Amino-2-methyl-1-propanol | |
| 0.20% Cremophor ® A 25 | |
| 0.10% Uvinul ® P 25 | PEG-25 PABA |
| q.s. Preservative | |
| q.s. Perfume Oil | |
| ad 100% Water | |

### Application Example 44

### Pump Sprays

| | INCI |
|---|---|
| 4.00% Polymer from Example 1 | |
| 1.00% Panthenol | |
| 0.10% Uvinul ® MS 40 | Benzophenone-4 |
| q.s. Preservative | |
| q.s. Perfume Oil | |
| ad 100% Water | |

### Application Example 45

### Pump Sprays

| | INCI |
|---|---|
| 4.00% Polymer from Example 18 | |
| 1.00% Panthenol | |
| 0.10% Uvinul ® M 40 | Benzophenone-3 |
| q.s. Preservative | |
| q.s. Perfume Oil | |
| ad 100% Ethanol | |

### Application Example 46

### Hair Sprays

| | INCI |
|---|---|
| 5.00% Polymer from Example 17 | |
| 0.10% Silicone Oil Dow Corning ® DC 190 | Dimethicone |
| | Copolyol |
| 35.00% Dimethyl Ether | |
| 5.00% n-Pentane | |
| ad 100% Ethanol | |
| q.s. Perfume Oil | |

### Application Example 47

### Hair sprays VOC 55%

| | INCI |
|---|---|
| 1.00% Polymer from Example 1 | |
| 4.00% Luviset ® P.U.R. | Polyurethane-1 |
| 40.00% Dimethyl Ether | |
| 15.00% Ethanol | |
| q.s. Perfume Oil | |
| ad 100% Water | |

### Application Example 48

### Thickener for Hair Gels

0.50% Polymer from Example 17 neutralized with triethanolamine to pH 7.5

3.00% Luviset Clear

0.50% Panthenol

q.s. Perfume Oil

q.s Preservative

ad 100% Water

This gives a virtually clear hair gel with a viscosity of 26 200 mpa*s

The example can be repeated with the polymers of Examples 1 to 16 and 18 to 50.

### Application Example 49

### Thickener for hair gels

0.50% Polymer from Example 17 neutralized with triethanolamine to pH 7.5

6.00% Luviskol K 30

0.10% Phytantriol

0.50% Panthenol

q.s. Perfume Oil

q.s Preservative

ad 100% Water

This gives a virtually clear hair gel with a viscosity of 29 300 mpa*s
The example can be repeated with the polymers of Examples 1 to 16 and 18 to 50.

### Application Example 50

### Thickener for Hair Gels

| | INCI |
|---|---|
| 0.50% Polymer from Example 17 neutralized | |
| with triethanolamine to pH 7.5 | |
| 3.00% Luviskol ® K90 | |
| 0.50% Panthenol | |
| 0.10% Uvinul ® MS 40 | Benzophenone-3 |
| q.s. Perfume Oil | |
| q.s Preservative | |
| ad 100% Water | |

This gives a virtually clear hair gel with a viscosity of 29 200 mpa*s

The example can be repeated with the polymers of Examples 1 to 16 and 18 to 50.

### Application Example 51

### Thickener for Hair Gels

INCI

0.50% Polymer from Example 17 neutralized
with triethanolamine to pH 7.5
2.00% Luviskol ® K90
2.00% Luviskol ® VA64W
0.50% Panthenol
q.s. Perfume Oil
q.s Preservative
ad 100% Water

This gives a virtually clear hair gel with a viscosity of 20 500 mpa*s

The example can be repeated with the polymers of Examples 1 to 16 and 18 to 50.

### Application Examples 52

### Gel Cream with UV Filter

| | 1 | 2 | 3 | 4 |
|---|---|---|---|---|
| Acrylate/C10-30 Alkyl Acrylate | 0.40 | 0.35 | 0.40 | 0.35 |
| Crosspolymer | | | | |
| Polymer from Example 17 | 1.10 | 2.50 | 1.60 | 0.80 |
| Xanthan Gum | 0.10 | | 0.10 | |
| Cetearyl Alcohol | 3.00 | 2.50 | 3.00 | 2.50 |
| C12-15 Alkyl Benzoate | 4.00 | 4.50 | 4.00 | 4.50 |
| Caprylic/Capric Triglyceride | 3.00 | 3.50 | 3.00 | 3.50 |
| Uvinul ® A Plus | 2.00 | 1.50 | 0.75 | 1.00 |
| UVASorb ® K2A | 0.20 | 3.00 | | |
| Ethylhexyl Methoxycinnamate | 3.00 | | 1.00 | |
| Bis-Ethylhexyloxyphenol | 0.30 | 1.50 | | 2.00 |
| Methoxyphenyl Triazine | | | | |
| Butylmethoxydibenzoylmethane | 0.40 | | 2.00 | |
| Disodium Phenyldibenzimidazole- | 2.50 | | 0.50 | 2.00 |
| tetrasulfonate | | | | |
| Ethyhexyltriazone | 4.00 | | 3.00 | 4.00 |
| Octocrylene | 0.50 | 4.00 | | |
| Diethylhexylbutamidotriazone | 1.00 | | | 2.00 |
| Phenylbenzimidazolesulfonic acid | 0.50 | | 3.00 | |
| Methylenebis-benzotriazolyl | 2.00 | | 0.50 | 1.50 |
| Tetramethylbutylphenol | | | | |
| Ethylhexyl Salicylate | 0.30 | | 3.00 | |
| Drometrizole Trisiloxane | 0.30 | | 0.50 | |
| Terephthalidenedicamphorsulfonic | 0.40 | 1.50 | | 1.00 |
| acid | | | | |
| Diethylhexyl 2,6-naphthalate | 3.50 | 4.00 | 7.00 | 9.00 |
| Titanium Dioxide Microfine | 1.00 | | 3.00 | |
| Zinc Oxide Microfine | 1.00 | 3.00 | | 4.25 |
| Cyclic Dimethylpolysiloxane | 5.00 | 5.50 | 5.00 | 5.50 |
| Dimethicone Polydimethylsiloxane | 1.00 | 0.60 | 1.00 | 0.60 |
| Glycerol | 1.00 | 1.20 | 1.00 | 1.20 |
| Sodium Hydroxide | q.s. | q.s. | q.s. | q.s. |
| Preservative | 0.30 | 0.23 | 0.30 | 0.23 |
| Perfume | 0.20 | | 0.20 | |
| Water | ad 100 | ad 100 | ad 100 | ad 100 |

pH adjusted to 6.0

The example can be repeated with the polymers of the other Examples 1 to 50.

### Application Examples 53

### O/W Sunscreen Formulations

| | 1 | 2 | 3 | 4 | 5 | 6 | 7 |
|---|---|---|---|---|---|---|---|
| Glycerol Monostearate SE | 0.50 | 1.00 | 3.00 | | | 1.50 | |
| Glyceryl Stearate Citrate | 2.00 | | 1.00 | 2.00 | 4.00 | | |
| Stearic acid | | 3.00 | | 2.00 | | | |
| PEG-40 Stearate | 0.50 | | | | | 2.00 | |
| Cetyl Phosphate | | | | | | 1.00 | |
| Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Polymer from Example 1 | | 2.50 | 1.60 | 0.80 | 1.40 | 4.00 | 1.00 |
| Uvinul ® A Plus | 2.00 | 1.50 | 0.75 | 1.00 | 2.10 | 4.50 | 5.00 |
| UVASorb ® K2A | 0.20 | | | | | | |
| Ethylhexyl Methoxycinnamate | 0.30 | | | | 5.00 | 6.00 | 8.00 |
| Bis-Ethylhexyloxyphenol | 0.40 | 1.50 | | 2.00 | 2.50 | | 2.50 |
| Methoxyphenyltriazine | | | | | | | |
| Butylmethoxydibenzoylmethane | 0.60 | | 2.00 | | 2.00 | 1.50 | |
| Disodiumphenyldibenzimidazoletetrasulfonate | 0.90 | | 0.50 | 2.00 | | 0.30 | |
| Ethyhexyltriazone | 4.00 | | 3.00 | 4.00 | | 2.00 | |
| Octocrylene | | 4.00 | | | | | 7.50 |
| Diethylhexylbutamidotriazone | 1.00 | | | 2.00 | 1.00 | | 1.00 |
| Phenylbenzimidazolesulfonic acid | 0.50 | | 3.00 | | | | |
| Methylenebisbenzotriazolyl | 2.00 | | 0.50 | 1.50 | 2.50 | | |
| Tetramethylbutylphenol | | | | | | | |
| Ethylhexyl Salicylate | 0.30 | | 3.00 | | | | 5.00 |
| Drometrizole Trisiloxane | 0.80 | | 0.50 | | | 1.00 | |
| Terephthalidenedicamphorsulfonic acid | 0.50 | 1.50 | | 1.00 | 1.00 | | 0.50 |
| Diethylhexyl 2,6-naphthalate | 3.50 | | 7.00 | | 6.00 | 9.00 | |
| Titanium Dioxide Microfine | 1.00 | | 3.00 | | 3.50 | | 1.50 |
| Zinc Oxide Microfine | 2.00 | | | 0.25 | | 2.00 | |
| C12-15 Alkyl Benzoate | | 0.25 | | | 4.00 | 7.00 | |
| Dicapryl Ether | | | 3.50 | | 2.00 | | |
| Butylene Glycol Dicaprylate/Dicaprate | 5.00 | | 6.00 | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Shea Butter | | 2.00 | | | | | |
| PVP Hexadecene Copolymer | 0.20 | | | 0.50 | | 1.00 | |
| Glycerol | 3.00 | 7.50 | | 7.50 | 5.00 | | 2.50 |
| Xanthan Gum | 0.15 | | | | | | |
| Vitamin E Acetate | 0.60 | | 0.23 | | 0.70 | 1.00 | |
| Fucogel ® 1000 | | 3.00 | 10.00 | | | | |
| Soybean oil | | | | 0.50 | | 1.50 | 1.00 |
| Ethylhexyloxyglycine | 0.30 | | | | | | |
| DMDM Hydantoin | | 0.60 | 0.40 | 0.20 | | | |
| Glyacil-L | | | | 0.18 | 0.20 | | |
| Methyl Paraben | 0.15 | | 0.25 | | 0.50 | | |
| Phenoxyethanol | 1.00 | 0.40 | | | 0.40 | 0.50 | 0.40 |
| EDTA | 0.02 | | 0.05 | | | | |
| Ethanol | 2.00 | 1.50 | | 3.00 | | 1.20 | 5.00 |
| Perfume | 0.10 | 0.25 | 0.30 | | 0.40 | 0.20 | |
| Water | ad 100 | ad 100 | ad 100 | ad 100 | ad 100 | ad 100 | ad 100 |

The example can be repeated with the polymers of the other Examples 1 to 50.

### Application Examples 54

### Hydrodispersions

| | 1 | 2 | 3 | 4 | 5 |
|---|---|---|---|---|---|
| Ceteareth-20 | 1.00 | | | 0.50 | |
| Cetyl Alcohol | | | 1.00 | | |
| Sodium Carbomer | | 0.20 | | | |
| Acrylate/C10-30 Alkyl Acrylate Crosspolymer | 0.50 | | 0.40 | 0.10 | 0.50 |
| Xanthan Gum | | | | 0.15 | |
| Polymer from Example 18 | 3.00 | 2.00 | 2.50 | 6.00 | 0.80 |
| Uvinul ® A Plus | 2.00 | 1.50 | 0.75 | 1.00 | 2.10 |
| UVASorb ® K2A | 0.20 | 3.50 | | | |
| Ethylhexyl Methoxycinnamate | 0.30 | | | | 5.00 |
| Bis-Ethylhexyloxyphenol Methoxyphenyl- | 0.40 | 1.50 | | 2.00 | 2.50 |
| triazine | | | | | |
| Butylmethoxydibenzoylmethane | 0.30 | | 2.00 | | 2.00 |
| Disodium Phenyldibenzimidazole- | 0.40 | | 0.50 | 2.00 | |
| tetrasulfonate | | | | | |
| Ethyhexyltriazone | 4.00 | | 3.00 | 4.00 | |
| Octocrylene | 1.00 | 4.00 | | | |
| Diethylhexylbutamidotriazone | 0.30 | | | 2.00 | 1.00 |
| Phenylbenzimidazolesulfonic acid | 0.50 | | 3.00 | | |
| Methylenebisbenzotriazolyl | 2.00 | | 0.50 | 1.50 | 2.50 |
| Tetramethylbutylphenol | | | | | |
| Ethylhexyl Salicylate | 0.20 | | 3.00 | | |
| Drometrizole Trisiloxane | 0.40 | | 0.50 | | |
| Terephthalidenedicamphorsulfonic acid | 0.30 | 1.50 | | 1.00 | 1.00 |
| Diethylhexyl 2,6-naphthalate | | | 7.00 | | 9.00 |
| Titanium Dioxide Microfine | 1.00 | | 3.00 | | 3.50 |
| Zinc Oxide Microfine | 2.00 | | | 4.25 | |
| C12-15 Alkyl Benzoate | 2.00 | 2.50 | | | |
| Dicapryl Ether | | 4.00 | | | |
| Butylene Glycol Dicaprylate/Dicaprate | 4.00 | | 2.00 | 6.00 | |
| Dicapryl Carbonate | | 2.00 | 6.00 | | |
| Dimethicone | | 0.50 | 1.00 | | |
| Phenyltrimethicone | 2.00 | | 0.50 | | |
| Shea Butter | | 2.00 | | 5.00 | |
| PVP Hexadecene Copolymer | 0.50 | | | 0.50 | 1.00 |
| Tricontanyl PVP | 0.50 | | 1.00 | | |
| Ethylhexylglycerol | | | 1.00 | | 0.80 |
| Glycerol | 3.00 | 7.50 | | 7.50 | 8.50 |
| Vitamin E Acetate | 0.50 | | 0.25 | | 1.00 |
| Alpha-Glucosylrutin | 0.60 | | | 0.25 | |
| Fucogel ® 1000 | | 2.50 | 0.50 | | 2.00 |
| DMDM Hydantoin | | 0.60 | 0.45 | 0.25 | |
| Glyacil-S | 0.20 | | | | |
| Methyl Paraben | 0.50 | | 0.25 | 0.15 | |
| Phenoxyethanol | 0.50 | 0.40 | | 1.00 | |
| EDTA | | 0.01 | 0.05 | | 0.10 |
| Ethanol | 3.00 | 2.00 | 1.50 | | 7.00 |
| Perfume | 0.20 | | 0.05 | 0.40 | |
| Water | ad 100 | ad 100 | ad 100 | ad 100 | ad 100 |

The example can be repeated with the polymers of the other Examples 1 to 50.

### Application Examples 55

### W/O Sunscreen Formulations

| | 1 | 2 | 3 | 4 | 5 |
|---|---|---|---|---|---|
| Cetyldimethicone Copolyol | | 2.50 | | 4.00 | |
| Polyglyceryl-2 dipolyhydroxystearate | 5.00 | | | | 4.50 |
| PEG-30 Dipolyhydroxystearate | | | 5.00 | | |
| Polymer from Example 17 | 2.00 | 3.00 | 2.50 | 1.70 | 3.00 |
| Uvinul ® A Plus | 2.00 | 1.50 | 0.75 | 1.00 | 2.10 |
| UVASorb ® K2A | 0.30 | 2.00 | | | |
| Ethylhexyl Methoxycinnamate | 0.40 | | | | 5.00 |
| Bis-Ethylhexyloxyphenol Methoxyphenyl Triazine | 0.50 | 1.50 | | 2.00 | 2.50 |
| Butylmethoxydibenzoylmethane | 0.30 | | 2.00 | | 2.00 |
| Disodium Phenyldibenzimidazoletetrasulfonate | 2.50 | | 0.50 | 2.00 | |
| Ethyhexyltriazone | 0.40 | | 3.00 | 4.00 | |
| Octocrylene | 0.40 | 4.00 | | | |
| Diethylhexylbutamidotriazone | 1.00 | | | 2.00 | 1.00 |
| Phenylbenzimidazolesulfonic acid | 0.50 | | 3.00 | | |
| Methylenebisbenzotriazolyl | 0.50 | | 0.50 | 1.50 | 2.50 |
| Tetramethylbutylphenol | | | | | |
| Ethylhexyl Salicylate | 0.30 | | 3.00 | | |
| Drometrizole Trisiloxane | 0.80 | | 0.50 | | |
| Terephthalidenedicamphorsulfonic acid | 0.50 | 1.50 | | 1.00 | 1.00 |
| Diethylhexyl 2,6-naphthalate | | | 7.00 | | 4.00 |
| Titanium Dioxide Microfine | 1.00 | | 3.00 | | 3.50 |
| Zinc Oxide Microfine | 1.00 | | | 5.25 | |
| Paraffin Oil | 6.00 | 12.00 | 10.00 | | 8.00 |
| C12-15 Alkyl Benzoate | | | | 9.00 | |
| Dicaprylyl Ether | 10.00 | | | | 7.00 |
| Butylene Glycol Dicaprylate/Dicaprate | | | 2.00 | 8.00 | 4.00 |
| Dicaprylyl Carbonate | 5.00 | | 6.00 | | |
| Dimethicone | | 4.00 | 1.00 | 5.00 | |
| Cyclomethicone | 2.00 | 25.00 | | | 2.00 |
| Shea Butter | | | 3.00 | | |
| Vaseline | | 4.50 | | | |
| PVP Hexadecene Copolymer | 0.50 | | | 0.50 | 1.00 |
| Ethylhexylglycerol | | 0.30 | 1.00 | | 0.50 |
| Glycerol | 3.00 | 7.50 | | 7.50 | 8.50 |
| MgSO₄ | 1.00 | 0.50 | | 0.50 | |
| MgCl₂ | | | 1.00 | | 0.70 |
| Vitamin E Acetate | 0.50 | | 0.25 | | 1.00 |
| Ascorbyl Palmitate | 0.50 | | | 2.00 | |
| Fucogel ® 1000 | | | | 3.50 | 1.00 |
| DMDM Hydantoin | | 0.60 | 0.40 | 0.20 | |
| Methyl Paraben | 0.50 | | 0.25 | 0.15 | |
| Phenoxyethanol | 0.50 | 0.40 | | 1.00 | |
| EDTA | 0.12 | 0.05 | | 0.30 | |
| Ethanol | 3.00 | | 1.50 | | 5.00 |
| Perfume | 0.20 | | 0.40 | 0.35 | |
| Water | ad 100 | ad 100 | ad 100 | ad 100 | ad 100 |

The example can be repeated with the polymers of the other Examples 1 to 50.

### Application Examples 56

### PIT Emulsions

| | 1 | 2 | 3 | 4 | 5 | 6 | 7 | 8 |
|---|---|---|---|---|---|---|---|---|
| Glycerol Monostearate SE | 0.50 | 2.00 | 3.00 | 5.00 | | 0.50 | 4.00 | |
| Glyceryl Isostearate | | | | | 3.50 | 4.00 | 2.00 | |
| Isoceteth-20 | | 0.50 | | | 2.00 | | | |
| Ceteareth-12 | | 5.00 | | 1.00 | | | 3.50 | 5.00 |
| Ceteareth-20 | | 5.00 | | 1.00 | | | | 3.50 |
| PEG-100 Stearate | | | | 2.80 | | 2.30 | 3.30 | |
| Cetyl Alcohol | 5.20 | | 1.20 | 1.00 | 1.30 | | 0.50 | 0.30 |
| Cetyl Palmitate | 2.50 | 1.20 | | 1.50 | | 0.50 | | 1.50 |
| Cetyldimethicone Copolyol | | | | 0.50 | | 1.00 | | |
| Polyglyceryl-2 Dipolyhydroxystearate | | | | 0.75 | 0.30 | | | |
| Polymer from Example 1 | 3.20 | 5.00 | 2.50 | 3.00 | 2.00 | 1.70 | 2.90 | 3.50 |
| Uvinul ® A Plus | 0.20 | 1.50 | 0.75 | 1.00 | 2.10 | 4.50 | 5.00 | 2.10 |
| UVASorb ® K2A | 0.30 | | 4.00 | | | | 1.50 | |
| Ethylhexyl Methoxycinnamate | 0.40 | | | | 5.00 | 6.00 | 8.00 | 5.00 |
| Bis-Ethylhexyloxyphenol | 0.40 | 1.50 | | 2.00 | 2.50 | | 2.50 | 2.50 |
| Methoxyphenyltriazine | | | | | | | | |
| Butylmethoxydibenzoylmethane | 2.00 | | 2.00 | | 2.00 | 1.50 | | 2.00 |
| Disodium Phenyldibenzimidazole- | 2.50 | | 0.50 | 2.00 | | 0.30 | | |
| tetrasulfonate | | | | | | | | |
| Ethylhexyltriazone | 4.00 | | 3.00 | 4.00 | | 2.00 | | |
| Octocrylene | 2.00 | 4.00 | | | | | 7.50 | |
| Diethylhexylbutamidotriazone | 1.00 | | | 2.00 | 1.00 | | 1.00 | 1.00 |
| Phenylbenzimidazolesulfonic acid | 0.50 | | 3.00 | | | | | |
| Methylenebisbenzotriazolyl | 2.00 | | 0.50 | 1.50 | 2.50 | | | 2.50 |
| Tetramethylbutylphenol | | | | | | | | |
| Ethylhexyl Salicylate | 0.30 | | 3.00 | | | | 5.00 | |
| Drometrizole Trisiloxane | 0.30 | | 0.50 | | | 1.00 | | |
| Terephthalidenedicamphorsulfonic | 0.20 | 1.50 | | 1.00 | 1.00 | | 0.50 | 1.00 |
| acid | | | | | | | | |
| Diethylhexyl 2,6-naphthalate | | | 7.00 | | 10.00 | 7.50 | | 8.00 |
| Titanium Dioxide Microfine | 1.00 | | 3.00 | | 3.50 | | 1.50 | 3.50 |
| Zinc Oxide Microfine | | | | 0.25 | | 2.00 | | |
| C12-15 Alkyl Benzoate | 3.50 | | | 6.35 | | | | 0.10 |
| Cocoglycerides | | 3.00 | | 3.00 | | | | 1.00 |
| Dicapryl Ether | 4.50 | | | | | | | |
| Dicaprylyl Carbonate | | 4.30 | | 3.00 | | | | 7.00 |
| Dibutyl Adipate | | | | 0.50 | | | | 0.30 |
| Phenyltrimethicone | 2.00 | | | 3.50 | | 2.00 | | |
| Cyclomethicone | | 3.00 | | | | | | |
| Ethyl Galactomannan | | 0.50 | | | 2.00 | | | |
| Hydrogenated Cocoglycerides | | | | | 3.00 | 4.00 | | |
| Abil ® Wax 2440 | | | | | | 1.50 | 2.00 | |
| PVP Hexadecene Copolymer | | | | 1.00 | 1.20 | | | |
| Glycerol | 4.00 | 6.00 | 5.00 | | 8.00 | 10.00 | | |
| Vitamin E Acetate | 0.20 | 0.30 | 0.40 | | 0.30 | | | |
| Shea Butter | | 2.00 | | 3.60 | | 2.00 | | |
| Iodopropyl Butylcarbamate | 0.12 | | | | 0.20 | | | |
| Fucogel ® 1000 | | | | 0.10 | | | | |
| DMDM Hydantoin | 0.10 | | | | 0.12 | | 0.13 | |
| Methyl Paraben | | 0.50 | 0.30 | | 0.35 | | | |
| Phenoxyethanol | 0.50 | 0.40 | | 1.00 | | | | |
| Octoxyglycerol | | 0.30 | | | 1.00 | | 0.35 | |
| Ethanol | 2.00 | | 2.00 | | | 5.00 | | |
| EDTA | 0.40 | | 0.15 | | | 0.20 | | |
| Perfume | 0.20 | | 0.20 | | 0.24 | 0.16 | 0.10 | 0.10 |
| Water | ad 100 | ad 100 | ad 100 | ad 100 | ad 100 | ad 100 | ad 100 | ad 100 |

The example can be repeated with the polymers ot the other Examples 1 to 50.

### Application Examples 57

### Gel Creams

| | 1 | 2 | 3 | 4 |
|---|---|---|---|---|
| Acrylate/C10-30 Alkyl Acrylate | 0.40 | 0.35 | 0.40 | 0.35 |
| Crosspolymer | | | | |
| Luvigel ® EM | 1.50 | | | |
| Polymer from Example 18 | 0.50 | 2.00 | 3.60 | 3.00 |
| Xanthan Gum | 0.10 | 0.13 | 0.10 | 0.13 |
| Cetearyl Alcohol | 3.00 | 2.50 | 3.00 | 2.50 |
| C12-15 Alkyl Benzoate | 4.00 | 4.50 | 4.00 | 4.50 |
| Caprylic/Capric Triglyceride | | 3.50 | 3.00 | 3.50 |
| Titanium Dioxide Microfine | 1.00 | | 1.50 | |
| Zinc Oxide Microfine | 1.00 | 2.00 | | 2.25 |
| Dihydroxy acetone | | | 3.00 | 5.00 |
| Cyclic Dimethylpolysiloxane | 5.00 | 5.50 | 5.00 | 5.50 |
| Dimethicone Polydimethylsiloxane | 1.00 | 0.60 | 1.00 | 0.60 |
| Glycerol | | 1.20 | 1.00 | 1.20 |
| Sodium Hydroxide | q.s. | q.s. | q.s. | q.s. |
| Preservative | 0.30 | 0.23 | 0.30 | 0.23 |
| Perfume | 0.20 | | 0.20 | |
| Water | ad 100 | ad 100 | ad 100 | ad 100 |

pH adjusted to 6.0

The example can be repeated with the polymers of the other Examples 1 to 50.

### Application Examples 58

### O/W Formulations

| | 1 | 2 | 3 | 4 | 5 | 6 | 7 |
|---|---|---|---|---|---|---|---|
| Glycerol Monostearate SE | 0.50 | 1.00 | 3.00 | | | 1.50 | |
| Glyceryl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Cetearyl Sulfate | | | | | | | 0.75 |
| Stearyl Alcohol | | | 3.00 | | | 2.00 | 0.60 |
| Cetyl Alcohol | | 1.10 | | 1.50 | 0.60 | | 2.00 |
| Polymer from Example 17 | 2.00 | 5.00 | 3.00 | 2.40 | 2.00 | 1.00 | 1.00 |
| Dihydroxyacetone | | | 3.00 | 5.00 | | 4 | |
| Titanium Dioxide Microfine | 1.00 | | | | 1.50 | | 1.50 |
| Zinc Oxide Microfine | 1.00 | | | 0.25 | | 2.00 | |
| C12-15 Alkyl Benzoate | | 0.25 | | | 4.00 | 7.00 | |
| Dicapryl Ether | | | 3.50 | | 2.00 | | |
| Butylene Glycol | 5.00 | | 6.00 | | | | |
| Dicaprylate/Dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Shea Butter | | 2.00 | | | | | |
| PVP Hexadecene Copolymer | 0.20 | | | 0.50 | | 1.00 | |
| Glycerol | 3.00 | 7.50 | | 7.50 | 5.00 | | 2.50 |
| Xanthan Gum | 0.15 | | 0.05 | | | 0.30 | |
| Sodium Carbomer | | | | 0.15 | | | |
| Vitamin E Acetate | 0.60 | | 0.23 | | 0.70 | 1.00 | |
| Fucogel ® 1000 | | 3.00 | 10.00 | | | | |
| Paraffin Oil | 4.00 | 8.00 | 5.00 | 7.00 | 3.90 | 4.00 | 1.00 |
| Ethylhexyloxyglycine | 0.30 | | | | | | |
| DMDM Hydantoin | | 0.60 | 0.40 | 0.20 | | | |
| Glyacil-L | | | | 0.18 | 0.20 | | |
| Methyl Paraben | 0.15 | | 0.25 | | 0.50 | | |
| Phenoxyethanol | 1.00 | 0.40 | | | 0.40 | 0.50 | 0.40 |
| EDTA | 0.02 | | 0.05 | | | | |
| Ethanol | 2.00 | 1.50 | | 3.00 | | 1.20 | 5.00 |
| Perfume | 0.10 | 0.25 | 0.30 | | 0.40 | 0.20 | |
| Water | ad 100 | ad 100 | ad 100 | ad 100 | ad 100 | ad 100 | ad 100 |

The example can be repeated with the polymers of the other Examples 1 to 50.

### Application Examples 59

### O/W Makeups

| | 1 | 2 | 3 | 4 | 5 | 6 | 7 |
|---|---|---|---|---|---|---|---|
| Glycerol Monostearate SE | 0.50 | 1.00 | 3.00 | | | 1.50 | |
| Glyceryl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Cetearyl Sulfate | | | | | | | 0.75 |
| Stearyl Alcohol | | | 3.00 | | | 2.00 | 0.60 |
| Cetyl Alcohol | | 1.10 | | 1.50 | 0.60 | | 2.00 |
| Polymer from Example 1 | 3.00 | 2.40 | 0.90 | 3.00 | 2.00 | 3.00 | 1.00 |
| Titanium Dioxide Microfine | 1.00 | 12.00 | 9.00 | 8.50 | 11.00 | 9.50 | 10.00 |
| Iron Oxides | 1.00 | 4.00 | 3.00 | 5.00 | 3.40 | 6.00 | 4.40 |
| Zinc Oxide Microfine | 1.00 | 4.00 | | 2.00 | | 3.00 | |
| C12-15 Alkyl Benzoate | | 0.25 | | | 4.00 | 7.00 | |
| Dicapryl Ether | | | 3.50 | | 2.00 | | |
| Butylene Glycol | 5.00 | | 6.00 | | | | |
| Dicaprylate/Dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Shea Butter | | 2.00 | | | | | |
| PVP Hexadecene Copolymer | 0.20 | | | 0.50 | | 1.00 | |
| Glycerol | 3.00 | 7.50 | | 7.50 | 5.00 | | 2.50 |
| Xanthan Gum | 0.15 | | 0.05 | | | 0.30 | |
| Sodium Carbomer | | 0.20 | | | | | |
| Vitamin E Acetate | 0.60 | | 0.23 | | 0.70 | 1.00 | |
| Paraffin Oil | 3.00 | 5.00 | 7.00 | 4.00 | 3.60 | 4.20 | 1.00 |
| Ethylhexyloxyglycine | 0.30 | | | | | | |
| DMDM Hydantoin | | 0.60 | 0.40 | 0.20 | | | |
| Glyacil-L | | | | 0.18 | 0.20 | | |
| Methyl Paraben | 0.15 | | 0.25 | | 0.50 | | |
| Phenoxyethanol | 1.00 | 0.40 | | | 0.40 | 0.50 | 0.40 |
| EDTA | 0.02 | | 0.05 | | | | |
| Iminosuccinic Acid | | | | 0.25 | 1.00 | | |
| Ethanol | 2.00 | 1.50 | | 3.00 | | 1.20 | 5.00 |
| Perfume | 0.10 | 0.25 | 0.30 | | 0.40 | 0.20 | |
| Water | ad 100 | ad 100 | ad 100 | ad 100 | ad 100 | ad 100 | ad 100 |

The example can be repeated with the polymers of the other Examples 1 to 50.

### Application Examples 60

### Hydrodispersions

| | 1 | 2 | 3 | 4 | 5 |
|---|---|---|---|---|---|
| Ceteareth-20 | 1.00 | | | 0.50 | |
| Cetyl Alcohol | | | 1.00 | | |
| Luvigel ® EM | | | | 2.50 | |
| Acrylate/C10-30 Alkyl Acrylate Crosspolymer | 0.50 | | 0.40 | 0.10 | 0.50 |
| Xanthan Gum | | | 0.30 | 0.15 | |
| Polymer from Example 18 | 3.00 | 2.00 | 0.90 | 0.40 | 3.00 |
| Dihydroxyacetone | | | 3.00 | 5.00 | |
| Uvinul ® A Plus | | 1.50 | 0.75 | 1.00 | 2.10 |
| Titanium Dioxide Microfine | 1.00 | | 1.00 | | 1.00 |
| Zinc Oxide Microfine | 1.00 | 1.90 | | 0.25 | |
| C12-15 Alkyl Benzoate | | 2.50 | | | |
| Dicapryl Ether | | 4.00 | | | |
| Butylene Glycol Dicaprylate/Dicaprate | 4.00 | | 2.00 | 6.00 | |
| Dicapryl Carbonate | | 2.00 | 6.00 | | |
| Dimethicone | | 0.50 | 1.00 | | |
| Phenyltrimethicone | 2.00 | | 0.50 | | |
| Shea Butter | | 2.00 | | 5.00 | |
| PVP Hexadecene Copolymer | 0.50 | | | 0.50 | 1.00 |
| Tricontanyl PVP | 0.50 | | 1.00 | | |
| Ethylhexylglycerol | | | 1.00 | | 0.80 |
| Glycerol | 3.00 | 7.50 | | 7.50 | 8.50 |
| Paraffin Oil | 3.00 | 5.00 | 7.00 | 4.00 | 3.60 |
| Vitamin E Acetate | 0.50 | | 0.25 | | 1.00 |
| Alpha-Glucosylrutin | 0.60 | | | 0.25 | |
| DMDM Hydantoin | | 0.60 | 0.45 | 0.25 | |
| Glyacil-S | 0.20 | | | | |
| Methyl Paraben | 0.50 | | 0.25 | 0.15 | |
| Phenoxyethanol | 0.50 | 0.40 | | 1.00 | |
| EDTA | | 0.01 | 0.05 | | 0.10 |
| Ethanol | 3.00 | 2.00 | 1.50 | | 7.00 |
| Perfume | 0.20 | | 0.05 | 0.40 | |
| Water | ad 100 | ad 100 | ad 100 | ad 100 | ad 100 |

The example can be repeated with the polymers of the other Examples 1 to 50.

### Application Examples 61

### Aftersun Hydrodispersions

| | 1 | 2 | 3 | 4 | 5 |
|---|---|---|---|---|---|
| Ceteareth-20 | 1.00 | | | 0.50 | |
| Cetyl Alcohol | | | 1.00 | | |
| Luvigel ® EM | | 2.00 | | | |
| Acrylate/C10-30 Alkyl Acrylate Crosspolymer | 0.50 | 0.30 | 0.40 | 0.10 | 0.50 |
| Xanthan Gum | | 0.30 | 0.15 | | |
| Polymer from Example 17 | 3.00 | 0.50 | 2.00 | 2.00 | 3.00 |
| C12-15 Alkyl Benzoate | 2.00 | 2.50 | | | |
| Dicapryl Ether | | 4.00 | | | |
| Butylene Glycol Dicaprylate/Dicaprate | | | 2.00 | 6.00 | |
| Dicapryl Carbonate | | 2.00 | 6.00 | | |
| Dimethicone | | 0.50 | 1.00 | | |
| Phenyltrimethicone | | | 0.50 | | |
| Tricontanyl PVP | 0.50 | | 1.00 | | |
| Ethylhexylglycerol | | | 1.00 | | 0.80 |
| Glycerol | | 7.50 | | 7.50 | 8.50 |
| Paraffin Oil | 1.00 | 3.00 | 1.50 | 2.00 | 1.00 |
| Vitamin E Acetate | 0.50 | | 0.25 | | 1.00 |
| Alpha-Glucosylrutin | 0.60 | | | 0.25 | |
| EDTA | | 0.01 | 0.05 | | 0.10 |
| Ethanol | 15.00 | 10.00 | 8.00 | 12.00 | 9.00 |
| Perfume | 0.20 | | 0.05 | 0.40 | |
| Water | ad 100 | ad 100 | ad 100 | ad 100 | ad 100 |

The example can be repeated with the polymers of the other Examples 1 to 50.

### Application Examples 62

### W/O Emulsions

| | 1 | 2 | 3 | 4 | 5 |
|---|---|---|---|---|---|
| Cetyldimethicone | | 2.50 | | 4.00 | |
| Copolyol | | | | | |
| Polyglyceryl-2 | 5.00 | | | | 4.50 |
| Dipolyhydroxystearate | | | | | |
| PEG-30 | | | 5.00 | | |
| Dipolyhydroxystearate | | | | | |
| Polymer from Example 1 | 2.00 | 3.00 | 3.00 | 2.20 | 3.10 |
| Uvinul ® A Plus | 2.00 | 1.50 | 0.75 | 1.00 | 2.10 |
| Titanium Dioxide | 1.00 | | 3.00 | | 3.50 |
| Microfine | | | | | |
| Zinc Oxide | 1.00 | 0.90 | | 0.25 | |
| Microfine | | | | | |
| Paraffin Oil | 4.00 | 12.00 | 10.00 | 2.00 | 8.00 |
| C12-15 Alkyl Benzoate | | | | 9.00 | |
| Dicaprylyl Ether | 10.00 | | | | 7.00 |
| Butylene Glycol | | | 2.00 | 8.00 | 4.00 |
| Dicaprylate/Dicaprate | | | | | |
| Dicaprylyl Carbonate | | | 6.00 | | |
| Dimethicone | | 4.00 | 1.00 | 5.00 | |
| Cyclomethicone | 2.00 | 25.00 | | | 2.00 |
| Shea Butter | | | 3.00 | | |
| Vaseline | | 4.50 | | | |
| PVP Hexadecene | 0.50 | | | 0.50 | 1.00 |
| Copolymer | | | | | |
| Ethylhexylglycerol | | 0.30 | 1.00 | | 0.50 |
| Glycerol | 3.00 | 7.50 | | 7.50 | 8.50 |
| MgSO₄ | 1.00 | 0.50 | | 0.50 | |
| MgCl₂ | | | 1.00 | | 0.70 |
| Vitamin E Acetate | 0.50 | | 0.25 | | 1.00 |
| Ascorbyl Palmitate | 0.50 | | | 2.00 | |
| Fucogel ® 1000 | | | | 3.50 | 7.00 |
| DMDM Hydantoin | | 0.60 | 0.40 | 0.20 | |
| Methyl Paraben | 0.50 | | 0.25 | 0.15 | |
| Phenoxyethanol | 0.50 | 0.40 | | 1.00 | |
| EDTA | 0.12 | 0.05 | | 0.30 | |
| Perfume | 0.20 | | 0.40 | 0.35 | |
| Water | ad 100 | ad 100 | ad 100 | ad 100 | ad 100 |

The example can be repeated with the polymers ot the other Examples 1 to 50.

### Application Example 63

### Solids-Stabilized Emulsions

| | 1 | 2 | 3 | 4 | 5 |
|---|---|---|---|---|---|
| Paraffin Oil | 4.00 | 6.00 | 16.00 | 16.00 | 6.00 |
| Octyldodecanol | 9.00 | 9.00 | 5.00 | | |
| Caprylic/Capric Triglyceride | 9.00 | 9.00 | 6.00 | | |
| C12-15 Alkyl Benzoate | | | | 5.00 | 8.00 |
| Butylene Glycol Dicaprylate/Dicaprate | | | | | 8.00 |
| Dicaprylyl Ether | 9.00 | | | 4.00 | |
| Dicaprylyl Carbonate | | 9.00 | | | |
| Hydroxyoctacosanyl Hydroxystearate | 2.00 | 2.00 | 2.20 | 2.50 | 1.50 |
| Disteardimonium Hectorite | | 0.75 | | 0.50 | 0.25 |
| Cera Microcristallina + Paraffinum Liquidum | | 0.35 | | | 5.00 |
| Hydroxypropylmethylcellulose | | | 0.10 | | 0.05 |
| Dimethicone | | | | | 3.00 |
| Polymer from Example 18 | 3.00 | 5.00 | 0.90 | 1.40 | 2.00 |
| Titanium Dioxide + Alumina + Simethicone + Aqua | 1.00 | 3.00 | | | |
| Titanium Dioxide + Trimethoxycaprylylsilane | 1.00 | 2.00 | 4.00 | 2.00 | 4.00 |
| Zinc Oxide Z-Cote HP1 | 3.00 | | 2.00 | | |
| Silica Dimethyl Silylate | 2.50 | | | 6.00 | 2.50 |
| Boron Nitride | | | 1.00 | | |
| Starch/Sodium Metaphosphate Polymer | 2.00 | | | | |
| Tapioca Starch | | 0.50 | | | |
| Sodium Chloride | 5.00 | 7.00 | 8.50 | 3.00 | 4.50 |
| Glycerol | | | | 1.00 | |
| EDTA | 1.00 | 1.00 | 1.00 | 1.00 | 1.00 |
| Vitamin E Acetate | 5.00 | 10.00 | 3.00 | 6.00 | 10.00 |
| Ascorbyl Palmitate | 1.00 | 1.00 | | 1.00 | |
| Methyl Paraben | | 0.60 | | | 0.20 |
| Propyl Paraben | | | | | 0.20 |
| Phenoxyethanol | | | 0.20 | | |
| Hexamidine Diisethionate | | | 0.40 | 0.50 | 0.40 |
| Diazolidinylurea | | | | | 0.08 |
| Ethanol | | | 0.23 | 0.20 | |
| Perfume | 5.00 | | 3.00 | 4.00 | |
| Water | 0.20 | | 0.30 | 0.10 | |
| | ad 100 | ad 100 | ad 100 | ad 100 | ad 100 |

The example can be repeated with the polymers of the other Examples 1 to 50.

### Application Examples 64

### PIT Emulsions

| | 1 | 2 | 3 | 4 | 5 | 6 | 7 | 8 |
|---|---|---|---|---|---|---|---|---|
| Glycerol Monostearate SE | 0.50 | 2.00 | 3.00 | 5.00 | | 0.50 | 4.00 | |
| Glyceryl Isostearate | | | | | 3.50 | 4.00 | 2.00 | |
| Isoceteth-20 | | 0.50 | | | 2.00 | | | |
| Ceteareth-12 | | 5.00 | | 1.00 | | | 3.50 | 5.00 |
| Ceteareth-20 | | 5.00 | | 1.00 | | | | 3.50 |
| PEG-100 Stearate | 3.00 | | | 2.80 | | 2.30 | 3.30 | |
| Cetyl Alcohol | 5.20 | | 1.20 | 1.00 | 1.30 | | 0.50 | 0.30 |
| Cetyl Palmitate | 2.50 | 1.20 | | 1.50 | | 0.50 | | 1.50 |
| Cetyl Dimethicone Copolyol | | | | 0.50 | | 1.00 | | |
| Polyglyceryl-2 Dipolyhydroxystearate | 0.30 | | | 0.75 | 0.30 | | | |
| Polymer from Example 17 | 2.00 | 5.00 | 2.00 | 3.00 | 2.00 | 1.60 | 3.80 | 4.00 |
| Dihydroxyacetone | | | 3.00 | 5.00 | | | 4.00 | |
| Uvinul ® A Plus | 2.00 | 1.50 | 0.75 | 1.00 | 2.10 | 4.50 | 5.00 | 2.10 |
| Titanium Dioxide Microfine | 1.00 | | 1.50 | | 3.50 | | 1.50 | 1.00 |
| Zinc Oxide Microfine | 1.00 | 1.00 | | 0.25 | | 2.00 | | 1.50 |
| C12-15 Alkyl Benzoate | 3.50 | | | 6.35 | | | | 0.10 |
| Cocoglycerides | | 3.00 | | 3.00 | | | | 1.00 |
| Dicapryl Ether | 4.50 | | | | | | | |
| Dicaprylyl Carbonate | | 4.30 | | 3.00 | | | | 7.00 |
| Dibutyl Adipate | | | | 0.50 | | | | 0.30 |
| Phenyltrimethicone | 2.00 | | | 3.50 | | 2.00 | | |
| Cyclomethicone | | 3.00 | | | | | | |
| Ethyl Galactomannan | | 0.50 | | | 2.00 | | | |
| Hydrogenated Cocoglycerides | | | | | 3.00 | 4.00 | | |
| Abil ® Wax 2440 | | | | | | 1.50 | 2.00 | |
| PVP Hexadecene Copolymer | | | | 1.00 | 1.20 | | | |
| Glycerol | 4.00 | 6.00 | 5.00 | | 8.00 | 10.00 | | |
| Vitamin E Acetate | 0.20 | 0.30 | 0.40 | | 0.30 | | | |
| Shea Butter | | 2.00 | | 3.60 | | 2.00 | | |
| Iodopropyl Butylcarbamate | 0.12 | | | | 0.20 | | | |
| DMDM Hydantoin | 0.10 | | | | 0.12 | | 0.13 | |
| Methyl Paraben | | 0.50 | 0.30 | | 0.35 | | | |
| Phenoxyethanol | 0.50 | 0.40 | | 1.00 | | | | |
| Octoxyglycerol | | 0.30 | | | 1.00 | | 0.35 | |
| Ethanol | 2.00 | | 2.00 | | | 5.00 | | |
| EDTA | 0.40 | | 0.15 | | | 0.20 | | |
| Perfume | 0.20 | | 0.20 | | 0.24 | 0.16 | 0.10 | 0.10 |

The example can be repeated with the polymers of the other Examples 1 to 50.

### Application Examples 65

### O/W Foot Cream Formulations

| | 1 | 2 | 3 | 4 |
|---|---|---|---|---|
| INCI Name | w/w % | w/w % | w/w % | w/w % |
| Aqua | ad 100 | ad 100 | ad 100 | ad 100 |
| Glycerin | 5.00 | 6.00 | 4.50 | 3.50 |
| Polymer from Example 1 | 2.30 | 3.50 | 0.40 | 1.20 |
| Cetearyl Glucoside, Cetearyl | 8.00 | 7.00 | 5.00 | 8.00 |
| Alcohol | | | | |
| Persea Gratissima (Avocado) | 5.00 | 4.50 | 3.00 | 6.00 |
| Oil | | | | |
| Paraffin Oil | 6.00 | 3.00 | 5.00 | 5.00 |
| Triticum Vulgare (Wheat) Germ | 1.00 | 1.50 | 1.00 | 2.00 |
| Oil | | | | |
| Dicaprylyl Ether | 2.00 | 2.00 | 2.50 | 1.00 |
| Caprylic/Capric Triglyceride | 5.00 | 7.00 | 2.50 | 5.00 |
| Dimethicone | 0.10 | 0.20 | 0.25 | 0.15 |
| Panthenol | 1.00 | 1.00 | 1.00 | 1.00 |
| Tocopheryl Acetate | 1.00 | 1.00 | 1.00 | 1.00 |
| Phenoxyethanol, | 0.60 | 0.50 | 0.60 | 0.50 |
| Methylparaben, Ethylparaben, | | | | |
| Butylparaben, Propylparaben, | | | | |
| Isobutylparaben | | | | |

The example can be repeated with the polymers of the other Examples 1 to 50.

### Application Examples 66

### Triple-Active Body Balms

| | | 1 | 2 | 3 | 4 | 5 |
|---|---|---|---|---|---|---|
| INCI Name | w/w % | w/w % | w/w % | w/w % | w/w % | w/w % |
| Aqua | ad 100 | ad 100 | ad 100 | ad 100 | ad 100 | ad 100 |
| Glycerin | 3.0 | 5.50 | 6.00 | 4.50 | 5.00 | 3.5 |
| Glyceryl Stearate Citrate | 1.80 | 2.00 | 2.50 | 3.00 | 1.50 | 2 |
| Sucrose Stearate | 1.00 | 1.20 | 0.95 | 2.00 | 2.20 | 1.5 |
| Cetearyl Alcohol | 1.80 | 2.00 | 3.00 | 1.50 | 2.40 | 2.8 |
| Ethylhexyl Palmitate | 6.00 | 5.00 | 5.50 | 3.50 | 3.00 | 5.5 |
| Paraffin Oil | 5.00 | 5.00 | 4.00 | 1.00 | 2.00 | 3.5 |
| Cetearyl Isononanoate | 7.00 | 3.00 | 2.50 | 2.40 | 3.10 | 4.6 |
| Phenoxyethanol, | 1.00 | 0.60 | 0.50 | 0.70 | 0.60 | 0.5 |
| Methylparaben, Ethylparaben, | | | | | | |
| Propylparaben, Butylparaben | | | | | | |
| Dimethicone | 2.00 | 1.50 | 0.20 | 0.50 | 1.80 | 1.4 |
| Xanthan Gum | 0.25 | | | | | 0.2 |
| Polymer from Example 18 | 1.00 | 1.50 | 1.80 | 0.80 | 2.00 | 0.7 |
| Sodium Hydroxide, Aqua | 0.20 | 0.15 | 0.20 | 0.50 | 0.20 | 0.2 |
| Bisabolol | 2.00 | 1.50 | 0.50 | 0.20 | 0.50 | 1 |
| Aqua, Sodium Ascorbyl | 1.00 | 0.50 | 1.50 | 2.00 | 3.00 | 1.5 |
| Phosphate | | | | | | |
| RetiSTAR | 1.00 | 0.50 | 0.90 | 0.80 | 0.50 | 1 |
| Perfume | 0.05 | 0.10 | 0.05 | 0.10 | 0.05 | 0.05 |

The example can be repeated with the polymers of the other Examples 1 to 50.

### Application Examples 67

### Liposculpt Creams for Men

| | | 1 | 2 | 3 | 4 |
|---|---|---|---|---|---|
| Raw Material | % by wt. | % by wt. | % by wt. | % by wt. | % by wt. |
| Water deionised | ad 100 | ad 100 | ad 100 | ad 100 | ad 100 |
| Polymer from Example 17 | 1.00 | 1.50 | 2.00 | 2.20 | 1.80 |
| Butylene Glycol | 5.00 | 6.50 | 5.50 | 3.50 | 4.00 |
| Mixed parabens | 0.20 | 0.20 | 0.20 | 0.20 | 0.20 |
| Potassium Sorbate | 0.10 | 0.10 | 0.10 | 0.10 | 0.10 |
| Stearic Acid | 1.50 | 2.00 | 1.90 | 2.50 | 1.00 |
| Crodamol GTCC | 2.00 | 5.00 | 3.00 | 4.00 | 2.50 |
| (Caprylic/Capric Triglyceride) | | | | | |
| Crodacol C90 (Cetyl Alcohol) | 0.50 | 1.50 | 2.00 | 1.00 | 0.50 |
| Crodacol CES (Cetearyl Alcohol | 1.50 | 2.00 | 1.80 | 1.90 | 2.10 |
| & Dicetyl Phosphate & Ceteth | | | | | |
| 10 Phosphate) | | | | | |
| Paraffin Oil | 2.00 | 5.00 | 10.00 | 7.50 | 4.00 |
| Aqua, Sodium Hydroxide | 0.50 | 0.60 | 0.80 | 0.90 | 0.70 |
| Aqua, Sodium Ascorbyl | 3.00 | 5.00 | 6.00 | 2.00 | 1.50 |
| Phosphate | | | | | |
| Ethanol 95 | 3.00 | 5.00 | 4.00 | 6.00 | 5.50 |
| Fragrance | 0.10 | 0.05 | 0.05 | 0.10 | 0.15 |

The example can be repeated with the polymers of the other Examples 1 to 50.

### Application Examples 68

### Presun & Aftersun Creams

| | 1 | 2 | 3 | 4 | 5 |
|---|---|---|---|---|---|
| INCI | % by wt. | % by wt. | % by wt. | % by wt. | % by wt. |
| Aqua | ad 100 | ad 100 | ad 100 | ad 100 | ad 100 |
| Panthenol | 2.0 | 3 | 2.5 | 3.5 | 3 |
| Cetearyl Alcohol (and) Dicetyl | 5.00 | 6.00 | 5.50 | 4.00 | 4.50 |
| Phosphate (and) Ceteth-10 | | | | | |
| Phosphate | | | | | |
| Cocoglycerides | 6.00 | 5.00 | 4.00 | 1.00 | 2.00 |
| C12-15 Alkyl Benzoate | 3.00 | 2.00 | 1.00 | 2.50 | 5.00 |
| Decyl Cocoate | 3.00 | 4.00 | 1.00 | 5.00 | 5.00 |
| Squalane | 2.00 | 0.50 | 1.00 | 1.50 | 2.00 |
| Polymer from Example 1 | 1.00 | 2.50 | 2.00 | 1.50 | 0.50 |
| Paraffin Oil | 5.00 | 10.00 | 7.50 | 6.50 | 8.00 |
| Aqua, Sodium Hydroxide | 0.60 | 1.50 | 1.20 | 1.00 | 0.20 |
| Cyclopentasiloxane | 3.00 | 2.00 | 1.50 | 1.00 | 5.00 |
| Bisabolol | 0.20 | 0.50 | 0.15 | 0.30 | 0.45 |
| Phenoxyethanol, | 0.40 | 0.50 | 0.70 | 0.80 | 1.00 |
| Methylparaben, Butylparaben, | | | | | |
| Ethylparaben, Propylparaben, | | | | | |
| Isobutylparaben | | | | | |
| Aqua, Sodium Ascorbyl | 3.00 | 2.00 | 5.00 | 4.00 | 3.50 |
| Phosphate | | | | | |

The example can be repeated with the polymers of the other Examples 1 to 50.

### Application Examples 69

### Eve Fluids

| | 1 | 2 | 3 | 4 | 5 |
|---|---|---|---|---|---|
| INCI | % by wt. | % by wt. | % by wt. | % by wt. | % by wt. |
| Aqua | ad 100 | ad 100 | ad 100 | ad 100 | ad 100 |
| Galactoarabinan | 0.50 | 1.00 | 0.75 | 0.25 | 1.20 |
| Aqua, Hamamelis Virginiana | 10.00 | 5.00 | 3.00 | 2.00 | 4.50 |
| Niacinamide | 0.20 | 0.30 | 0.10 | 0.50 | 0.40 |
| Phenoxyethanol, | 0.50 | 0.70 | 0.80 | 1.00 | 0.50 |
| Methylparaben, Ethylparaben, | | | | | |
| Propylparaben, Butylparaben | | | | | |
| Paraffin Oil | 2.00 | 5.00 | 3.00 | 4.50 | 2.50 |
| Polymer from Example 18 | 2.50 | 3.00 | 1.50 | 2.00 | 2.00 |
| Cyclopentasiloxane, | 5.00 | 2.00 | 4.00 | 3.50 | 4.50 |
| Dimethicone/Vinyltrimethyl | | | | | |
| Siloxysilicate Crosspolymer | | | | | |
| Aqua, Caffeine | 2.00 | 5.00 | 3.00 | 4.00 | 3.00 |
| Aqua, Sodium Hydroxide | 1.20 | 2.50 | 1.80 | 2.40 | 1.80 |

The example can be repeated with the polymers of the other Examples 1 to 50.

### Application Examples 70

### Tingling Hair Styling Gels

| | 1 | 2 | 3 | 4 | 5 |
|---|---|---|---|---|---|
| INCI | % by wt. | % by wt. | % by wt. | % by wt. | % by wt. |
| Calcium Aluminium Borosilicate, | 2.55 | 2.50 | 2.30 | 2.00 | 3.00 |
| CI 77891 (Titanium Dioxide), | | | | | |
| Silica, Tin Oxide | | | | | |
| Alumina, CI 77891 (Titanium | 0.40 | 0.50 | 0.55 | 0.35 | 0.30 |
| Dioxide), Tin Oxide | | | | | |
| Mica, CI 77499 (Iron Oxides), CI | 0.05 | 0.10 | 0.15 | 0.20 | 0.05 |
| 77891 (Titanium Dioxide) | | | | | |
| Polymer from Example 17 | 4.50 | 5.50 | 6.00 | 6.50 | 7.00 |
| Isopropyl Alcohol | 20.00 | 15.00 | 25.00 | 30.00 | 40.00 |
| Aqua | 30.00 | 35.00 | 25.00 | 20.00 | 10.00 |
| PVP | 2.00 | 2.50 | 1.50 | 1.00 | 2.50 |
| Propylene Glycol, Diazolidinyl, | 1.00 | 0.80 | 1.00 | 1.00 | 1.00 |
| Urea, Methylparaben, | | | | | |
| Propylparaben | | | | | |
| AMP | 0.50 | 0.60 | 0.80 | 0.90 | 1.30 |
| Aqua | ad 100 | ad 100 | ad 100 | ad 100 | ad 100 |

The example can be repeated with the polymers of the other Examples 1 to 50.

### Application Examples 71

### W/O/W emulsions

| | 1 | 2 | 3 | 4 |
|---|---|---|---|---|
| Additive | % by wt. | % by wt. | % by wt. | % by wt. |
| Glyceryl Stearate | 3.00 | 5.00 | 4.50 | 3.50 |
| PEG-100 Stearate | 0.75 | 1.50 | 3.00 | 1.20 |
| Behenyl Alcohol | 2.00 | 1.50 | 2.50 | 4.00 |
| Caprylic/Capric | 8.00 | 10.00 | 6.50 | 5.00 |
| Triglyceride | | | | |
| Cetearyl Ethylhexanoate | 5.00 | 4.50 | 6.00 | 5.50 |
| C12-15 Alkyl Benzoate | 3.00 | 4.00 | 5.00 | 4.50 |
| Polymer from Example 1 | 5.00 | 4.50 | 3.50 | 3.00 |
| Ethylhexyl | 5.00 | 6.00 | 7.50 | 5.50 |
| Methoxycinnamate | | | | |
| Bis-Ethylhexyloxyphenol | 1.80 | | | |
| Methoxyphenyltriazine | | 2.00 | 1.50 | 1.00 |
| Ethylhexyltriazone | 1.50 | 1.00 | 0.50 | 1.20 |
| Magnesium Sulfate | 0.80 | 1.00 | 0.50 | 0.75 |
| (MgSO₄) | | | | |
| EDTA | 0.10 | 0.15 | 0.05 | 0.10 |
| Preservative | 0.50 | 0.60 | 0.80 | 1.00 |
| Perfume | 0.05 | 0.10 | 0.05 | 0.10 |
| Aqua, Triethanolamine | 5.50 | 5.00 | 4.50 | 4.00 |
| Water | ad 100.0 | ad 100.0 | ad 100.0 | ad 100.0 |

pH adjusted to 6.0

The example can be repeated with the polymers of the other Examples 1 to 50.

### Application Examples 72

### Deodorant Roll-Ons

| INCI | 1 | 2 | 3 | 4 | 5 |
|---|---|---|---|---|---|
| Polymer from | 1.5 | 2 | 2.5 | 3 | 2.2 |
| Example 18 | | | | | |
| Water | 30 | 30 | 30 | 30 | 30 |
| Aqua, Sodium Hydroxide | 0.6 | 0.8 | 1 | 1.4 | 0.9 |
| PEG-40 Hydrogenated | 2 | 2.5 | 3 | 3.5 | 3 |
| Castor Oil | | | | | |
| Bisabolol | 0.1 | 0.1 | 0.1 | 0.1 | 0.1 |
| Farnesol | 0.3 | 0.2 | 0.3 | 0.1 | 0.3 |
| Perfume | 0.1 | 0.05 | 0.2 | 0.1 | 0.05 |
| Water | ad 100 | ad 100 | ad 100 | ad 100 | ad 100 |
| Alcohol | 25 | 30 | 35 | 30 | 32 |
| Propylene Glycol | 3 | 2 | 2 | 3 | 2.5 |
| Allantoin | 0.1 | 0.1 | 0.1 | 0.1 | 0.1 |
| Aluminum Chlorohydrate | 5 | 5.5 | 7.5 | 6 | 5.5 |

The example can be repeated with the polymers of the other Examples 1 to 50.

### Application Examples 73

### Aftershave Balms

| | 1 | 2 | 3 | 4 | 5 |
|---|---|---|---|---|---|
| Ingredients | % | % | % | % | % |
| Acrylate/C₁₀₋₃₀ Alkyl | 0.25 | 0.30 | 0.34 | 0.25 | 0.40 |
| Acrylate Copolymer | | | | | |
| Tocopheryl Acetate | 1.50 | 2.00 | 2.50 | 0.50 | 1.00 |
| Bisabolol | 0.20 | 0.50 | 0.25 | 0.30 | 0.35 |
| Caprylic/Capric | 10.00 | 12.00 | 11.00 | 8.00 | 5.00 |
| Triglyceride | | | | | |
| Perfume | 0.05 | 0.10 | 0.10 | 0.15 | 0.10 |
| PEG-40 Hydrogenated | 1.00 | 1.50 | 2.00 | 2.50 | 1.50 |
| Castor Oil | | | | | |
| Panthenol | 1.00 | 1.50 | 2.00 | 0.80 | 0.75 |
| Alcohol | 15.00 | 20.00 | 25.00 | 23.00 | 19.00 |
| Glycerol | 5.00 | 4.00 | 6.00 | 7.50 | 8.00 |
| Hydroxyethylcellulose | 0.05 | 0.10 | 0.15 | 0.20 | 0.15 |
| Polymer from | 3.50 | 3.00 | 5.00 | 1.00 | 1.50 |
| Example 17 | | | | | |
| Dist. Water | ad 100 | ad 100 | ad 100 | ad 100 | ad 100 |
| Water, Sodium | 2.00 | 1.50 | 2.20 | 0.50 | 1.20 |
| Hydroxide | | | | | |

The example can be repeated with the polymers of the other Examples 1 to 50.

### Application Examples 74

### W/S Formulations

| | 1 | 2 | 3 | 4 | 5 |
|---|---|---|---|---|---|
| Ingredients | % | % | % | % | % |
| Cetyl PEG/PPG-10/1 | 2.50 | 3.50 | 3.00 | 2.00 | 2.50 |
| Dimethicone | | | | | |
| Cetyl Dimethicone | 1.50 | 1.00 | 2.50 | 3.00 | 4.00 |
| Dimethicone | 1.50 | 2.00 | 3.00 | 4.50 | 0.50 |
| Cyclopentasiloxane, | 15.00 | 20.00 | 25.00 | 10.00 | 18.00 |
| Cyclohexasiloxane | | | | | |
| Cyclopentasiloxane, Caprylyl | 1.50 | 2.50 | 5.00 | 0.50 | 3.50 |
| Dimethicone Ethoxy | | | | | |
| Glucoside | | | | | |
| Polymer from Example 1 | 1.50 | 2.00 | 0.50 | 1.30 | 0.80 |
| Phenyl Trimethicone | 1.50 | 2.00 | 5.00 | | |
| Sodium Chloride | 1.00 | 0.50 | 0.80 | 0.70 | 1.50 |
| Water, Triethanolamine | 1.00 | 2.00 | 0.70 | 1.10 | 1.00 |
| Propylene Glycol | 5.00 | 10.00 | 15.00 | 3.00 | 4.00 |
| Water | 70.00 | | | | |
| Phenoxyethanol, | 0.50 | | | 1.00 | 0.70 |
| Methylparaben, | | | | | |
| Ethylparaben, Butylparaben, | | | | | |
| Propylparaben | | | | | |

The example can be repeated with the polymers of the other Examples 1 to 50.

### Application Examples 75

### Water-Reduced Glycerol Gels, Fat-Free

| | 1 | 2 | 3 | 4 | 5 |
|---|---|---|---|---|---|
| Ingredients | % | % | % | % | % |
| Glycerol 99% | ad 100 | ad 100 | ad 100 | ad 100 | ad 100 |
| Polymer from | 0.50 | 1.00 | 1.50 | 2.00 | 2.00 |
| Example 18 | | | | | |
| Urea | 10.00 | 15.00 | 2.00 | 5.00 | 7.00 |
| Phenoxyethanol, | | 1.00 | | 0.55 | |
| Ethylhexylglycerol | | | | | |
| AMP | 0.30 | 0.60 | 0.90 | 1.20 | 1.20 |

The example can be repeated with the polymers of the other Examples 1 to 50.

### Application Examples 76

### Disinfectant Gels

| | 1 | 2 | 3 | 4 | 5 |
|---|---|---|---|---|---|
| Ingredient | % | % | % | % | % |
| Ethanol | 50.00 | 45.00 | 60.00 | 55.00 | 40.00 |
| Polymer from | 2.00 | 2.50 | 1.50 | 3.50 | 4.00 |
| Example 17 | | | | | |
| Farnesol | 0.50 | 0.80 | 0.30 | 0.40 | 0.55 |
| Chamomilla Recutita | 5.00 | 3.00 | 4.00 | 2.00 | 6.00 |
| (Matricaria) Flower/ | | | | | |
| Leaf Extract | | | | | |
| AMP | 1.20 | 1.50 | 0.90 | 2.10 | 2.40 |
| Water | ad 100 | ad 100 | ad 100 | ad 100 | ad 100 |

The example can be repeated with the polymers of the other Examples 1 to 50.

### Application Examples 77

### Deodorant Roll-Ons

| | 1 | 2 | 3 | 4 | 5 |
|---|---|---|---|---|---|
| Ingredients | % | % | % | % | % |
| Propylene Glycol | 50.00 | 60.00 | 70.00 | 65.00 | 75.00 |
| Aluminum Chlorohydrate | 20.00 | 15.00 | 20.00 | 20.00 | 15.00 |
| Allantoin | 0.05 | 0.15 | 0.10 | 0.15 | 0.20 |
| Polymer from Example 1 | 1.00 | 1.50 | 1.80 | 2.00 | 0.90 |
| AMP | 0.40 | 0.30 | 0.50 | 0.60 | 0.30 |
| Farnesol | 0.50 | 0.30 | 0.20 | 0.40 | 0.25 |
| Phenoxyethanol and | 1.00 | 0.90 | 0.80 | 1.00 | 1.00 |
| Benzoic Acid and | | | | | |
| Dehydroacetic Acid | | | | | |
| Water | ad 100 | ad 100 | ad 100 | ad 100 | ad 100 |

The example can be repeated with the polymers of the other Examples 1 to 50.

### Application Examples 78

### Water-Reduced Gels with Enzymes

| | 1 | 2 | 3 | 4 | 5 | 6 | 7 |
|---|---|---|---|---|---|---|---|
| | % | % | % | % | % | % | % |
| Glycerol 99% | ad 100 | ad 100 | ad 100 | ad 100 | ad 100 | ad 100 | ad 100 |
| Polymer from Example 18 | 2.50 | 3.00 | 1.50 | 1.00 | 3.00 | 1.00 | 3.00 |
| AMP | 1.50 | 1.80 | 0.60 | 0.60 | 1.80 | 0.60 | 1.80 |
| Preservative | q.s. | q.s. | q.s. | q.s. | q.s. | q.s. | q.s. |
| Ubiquinone | 0.01 | 0.05 | 0.1 | | | | |
| Subtilisin | | | | 0.01 | 0.01 | | |
| Plankton Extract, Lecithin | | | | | | 0.01 | 0.01 |

The example can be repeated with the polymers of the other Examples 1 to 50.

1. A process for modifying the rheology of aqueous, alcoholic or aqueous/alcoholic cosmetic or dermatological compositions, the process comprising:
adding polymers, of ethylenically unsaturated anionic monomers obtainable by free-radical polymerization of the monomers in an aqueous medium, to the rheology of aqueous, alcoholic or aqueous/alcoholic cosmetic or dermatological compositions,
wherein the polymerization is carried out in the presence of, in each case, at least one polymer chosen from group a) and at least one polymer chosen from group b),
wherein group a) consists of
a1) graft polymers of vinyl acetate and/or vinyl propionate on (i) polyethylene glycols or (ii) polyethylene glycols or polypropylene glycols terminally capped at one or both ends with alkyl, carboxyl or amino groups;
a2) polyalkylene glycols;
a3) polyalkylene glycols terminally capped at one or both ends with alkyl, carboxyl or amino groups; and
a4) copolymers of alkyl polyalkylene glycol (meth)acrylates and (meth)acrylic acid; and wherein group b) consists of
b1) at least partially hydrolyzed copolymers of vinyl alkyl ethers and maleic anhydride, which may be present at least partially in salt form;
b2) water-soluble starch chosen from the group consisting of cationically modified starch, anionically modified starch, degraded starch and maltodextrin;
b3) anionic copolymers chosen from the group of
homopolymers and copolymers of anionic monomers,
copolymers of anionic and cationic and, if appropriate, neutral monomers,
wherein the fraction of copolymerized anionic monomers is greater than that of cationic monomers, and
copolymers of at least one anionic monomer and at least one monomer from the group of esters of anionic monomers with monohydric alcohols, styrene, N-vinylpyrrolidone, N-vinylcaprolactam, N-vinylimidazole, N-vinylformamide, acrylamide, methacrylamide, vinyl acetate and vinyl propionate; and
b4) cationic copolymers of nonionic monoethylenically unsaturated monomers and cationic monoethylenically unsaturated monomers and, if appropriate, anionic monoethylenically unsaturated monomers, where in every case the number of cationic groups is greater than the number of anionic groups.

2. The process according to claim 1, wherein polyalkylene glycols with molar masses Mₙ of from 100 to 100 000, polyalkylene glycols terminally capped at one or both ends with alkyl, carboxyl or amino groups and having molar masses Mₙ of from 100 to 100 000 are used as polymers (a).

3. The process according to claim 1, wherein block copolymers of ethylene oxide and propylene oxide with a molar mass Mₙ of from 500 to 20 000 g/mol and a content of ethylene oxide units of from 10 to 80 mol % are used as polymers (a).

4. The process according to claim 1, wherein at least one homopolymer of an ethylenically unsaturated C₃- to C₅-carboxylic acid, vinylsulfonic acid, styrenesulfonic acid, acrylamidomethylpropanesulfonic acid, vinylphosphonic acid, salts thereof neutralized partially or completely with alkali metal and/or ammonium bases and/or at least one copolymer of these monomers are used as polymers (b).

5. The process according to claim 1, wherein partially or completely hydrolyzed copolymers of vinyl alkyl ethers and maleic anhydride, which, if appropriate, are present at least partially in the form of their alkali metal or ammonium salts, are used as polymers of group (b).

6. The process according to claim 1, wherein graft polymers of vinyl acetate on polyethylene glycols with a molecular weight Mₙ of from 1000 to 100 000 are used as polymers a), and partially or completely hydrolyzed copolymers of vinyl methyl ether and maleic anhydride, which may be present at least partially in the form of their alkali metal or ammonium salts, are used as polymers b).

7. The process according to claim 1, wherein copolymers of alkyl polyalkylene glycol (meth)acrylates and (meth)acrylic acid are used as polymers a), and at least one partially or completely hydrolyzed copolymer of vinyl methyl ether and maleic anhydride, which, if appropriate, is present at least partially in the form of their alkali metal or ammonium salts, is used as polymers b).

8. The process according to claim 1, wherein polypropylene glycols, polyethylene glycols and/or block copolymers of ethylene oxide and propylene oxide of molecular weight Mₙ from 300 to 50 000 and/or polypropylene glycols, polyethylene glycols and/or block copolymers of ethylene oxide and propylene oxide of molecular weight Mₙ from 300 to 50 000 terminally capped at one or both ends with C₁- to C₄-alkyl groups are used as polymers a) and maltodextrin is used as polymer b).

9. The process according to claim 1, wherein copolymers of
(i) at least one ethylenically unsaturated C₃- to C₅-carboxylic acid, vinylsulfonic acid, styrenesulfonic acid, acrylamidomethylpropanesulfonic acid, vinylphosphonic acid and/or alkali metal and/or ammonium salts thereof;
(ii) at least one cationic monomer chosen from the group consisting of partially or completely neutralized dialkylaminoalkyl (meth)acrylates, partially or completely quaternized dialkylaminoalkyl (meth)acrylates, dialkylaminoalkyl(meth)acrylamides in quaternized or neutralized form, dialkyldiallylammonium halides and quaternized n-vinylimidazole; and, if appropriate,
(iii) at least one neutral monomer, are used as polymers b),
wherein the fraction of copolymerized anionic monomers is greater than that of cationic monomers.

10. The process according to claim 1, wherein copolymers of
(i) at least one anionic monomer; and
(ii) at least one monomer from the group of esters of ethylenically unsaturated acids with monohydric alcohols, styrene, N-vinylpyrrolidone, N-vinylcaprolactam, N-vinylimidazole, N-vinylformamide, acrylamide, methacrylamide, vinyl acetate and vinyl propionate are used as polymers (b).

11. The process according to claim 1, wherein at least one block copolymer of ethylene oxide and propylene oxide is used as polymer a), and at least one copolymer which comprises acrylamide, dimethylaminoethyl acrylate methochloride and 0 to 5 mol % of acrylic acid in copolymerized form is used as polymer b).

12. The process according to claim 1, wherein copolymers of methacrylic acid and acrylamidomethylpropanesulfonic acid in which the molar ratio of the methacrylic acid used to produce the copolymers to acrylamidomethylpropanesulfonic acid is in the range from 9:1 to 1:9, are used as polymer b).

13. The process according to claim 1, wherein monoethylenically unsaturated C₃- to C₅-carboxylic acids, vinylsulfonic acid, styrenesulfonic acid, acrylamidomethylpropanesulfonic acid, vinylphosphonic acid and/or alkali metal or ammonium salts thereof are used as anionic monomers.

14. The process according to claim 1, wherein the polymerization of the anionic monomers is carried out in the presence of further ethylenically unsaturated monomers.

15. The process according to claim 1, wherein the polymerization of the anionic monomers is carried out in the presence of at least one monomer chosen from the group consisting of (meth)acrylamide, (meth)acrylic esters of monohydric C₁-C₂₂-alcohols, C₃-C₂₂-alkyl vinyl ethers, C₆-C₁₆-olefins, polyisobutene derivatives, vinyl acetate, vinyl propionate, dialkylaminoethyl (meth)acrylates, dialkylaminopropyl (meth)acrylates, diallyldimethylammonium chloride, N-vinylformamide, if appropriate quaternized vinylimidazoles and partially or completely neutralized or quaternized dialkylaminoinalkyl(meth)acrylamides.

16. The process according to claim 1, wherein acrylic acid is used in the absence of other monomers during the free-radical polymerization.

17. The process according to claim 1, wherein the polymerization is additionally carried out in the presence of at least one crosslinker.

18. The process according to claim 1, wherein the crosslinker is chosen from the group consisting of triallylamine, pentaerythritol triallyl ether, methylenebisacrylamide, N,N′-divinylethyleneurea, dihydric alcohols having 2 to 4 carbon atoms esterified completely with acrylic acid or methacrylic acid, ethoxylated trimethylolpropane triacrylates, ethoxylated trimethylolpropane trimethacrylates, pentaerythritol triacrylate, pentaerythritol tetraacrylate and/or triallylmethylammonium chloride, allyl ethers of sugars comprising at least two allyl groups, vinyl ethers having at least two vinyl groups, or triallylamine, and mixtures of these compounds.

19. A hair cosmetic composition comprising at least one polymer of ethylenically unsaturated anionic monomers as defined in claim 1.

20. A skin cosmetic composition comprising at least one polymer of ethylenically unsaturated anionic monomers as defined in claim 1.

21. A dermatological composition comprising at least one polymer of ethylenically unsaturated anionic monomers as defined in claim 1.
